(19) United States
(12) Patent Application Publication
(10) Pub. No.: US 2015/0218120 A1
(43) Pub. Date: Aug. 6, 2015

(54) HIV INTEGRASE INHIBITORS

(71) Applicants: The Regents of the University of California, Oakland, CA (US); National Institutes of Health, Bethesda, MD (US)

(72) Inventors: Seth M. Cohen, La Jolla, CA (US); Arpita Agrawal, La Jolla, CA (US); Jamie DeSoto, La Jolla, CA (US); Yves Pommier, La Jolla, CA (US); Kasthuraiah Maddali, Bethesda, CA (US)

(21) Appl. No.: 14/615,203

(22) Filed: Feb. 5, 2015

Related U.S. Application Data
(62) Division of application No. 13/957,715, filed on Aug. 2, 2013, now abandoned.
Application No. 13/957,715 is a continuation of application No. PCT/US2012/023662, filed on Feb. 2, 2012.
(60) Provisional application No. 61/438,887, filed on Feb. 2, 2011.
(60) Provisional application No. 61/589,846, filed on Jan. 23, 2012.

(51) Int. Cl. C07D 309/40; C07D 309/36; C07D 213/89; C07D 215/48; C07D 215/60; C07D 213/81; C07C 235/60

(52) U.S. Cl. CPC C07D 309/40; C07D 213/81; C07D 309/36; C07C 235/60; C07D 215/48; C07D 215/60; C07D 213/89

(57) ABSTRACT

Provided herein, inter alia, are novel compounds for the inhibition of HIV integrase. The compounds disclosed herein are useful for methods of treating HIV infection in a subject in need thereof.

### RELATED APPLICATIONS

This application is a continuation application of U.S. Nonprovisional application Ser. No. 13/957,715, filed Aug. 2, 2013, and claims priority to U.S. Provisional Patent Application No. 61/438,887, filed Feb. 2, 2011, entitled “HIV INTEGRASE INHIBITORS” and U.S. Provisional Patent Application No. 61/589,846, filed Jan. 23, 2012, entitled “HIV INTEGRASE INHIBITORS”. The disclosure of each of the above-referenced applications are incorporated by reference herein in their entirety.

### STATEMENT AS TO RIGHTS TO INVENTIONS MADE UNDER FEDERALLY SPONSORED RESEARCH OR DEVELOPMENT

This invention was made with government support under grant R01 HL00049-01 awarded by the National Institutes of Health. The Government has certain rights in the invention.

### BACKGROUND OF THE INVENTION

Human immunodeficiency virus (HIV) is a retrovirus that causes acquired immunodeficiency syndrome (AIDS) (Barre-Sinoussi F, et al., Science 220(4599):868-871 (1983); Schupbach J, et al., Science 224(4648):503-505 (1984)). There is presently no cure for AIDS, although potent antiretroviral drugs have improved the management of the disease (Mehellou Y & De Clercq E, J. Med. Chem. 53:521-538 (2010)). HIV integrase (HIV-1 IN) is one of three essential enzymes for HIV replication (along with HIV reverse transcriptase and protease). HIV-1 IN performs two functions related to inserting the viral genome into the host DNA. In its first function, known as 3′-processing, HIV-1 IN generates reactive CpA 3′-hydroxyl ends (cytosine-adenosine 3′ recessed ends) by specifically cleaving a dinucleotide from the viral cDNA. The second function of HIV-1 IN, known as strand transfer, occurs upon translocation to the nucleus, where HIV-1 IN uses the hydroxyl ends to integrate the viral DNA into the host genome (Pommier Y, Johnson A A, & Marchand C, Nat. Rev. Drug Dis. 4(3):236-248 (2005); Li X, et al., Virology 411(2):194-205 (2011)).

The active site of HIV-1 IN is characterized by a dinuclear magnesium center, coordinated by three carboxylate ligands in a DDE amino acid motif (Li X, et al., Virology 411(2):194-205 (2011); Chiu T K & Davies D R, Curr. Top. Med. Chem. 4(9):965-977 (2004); Perryman A L, et al., J. Mol. Biol. 397:600-615 (2010)). The metal-dependent activity of HIV-1 IN has proven to be exceptionally important in the development of inhibitors against this metalloenzyme. The FDA approved the first HIV-1 IN inhibitor, raltegravir, in 2007. Raltegravir utilizes a 5-hydroxy-3-methylpyrimidin-4(3H)-one (HMPO) chelating group in combination with an amide carbonyl oxygen atom to bind the dinuclear Mg²⁺ metal site in HIV-1 IN. The HMPO metal-binding group was discovered by high-throughput screening (HTS) and was found to possess suitable pharmacokinetics (Iwamoto M, et al., Clin. Pharmacol. Ther. 83:293-299 (2008); Marchand C, et al., Curr. Top. Med. Chem. 9:1016-1037 (2009); Summa V, et al., J. Med. Chem. 51(18):5843-5855 (2008)). The HMPO chelator and the amide carbonyl oxygen atom provide three, essentially co-planar oxygen atoms to bind and bridge the Mg²⁺ ions of HIV-1 IN (FIG. 1). Despite the success of raltegravir, resistant HIV strains have emerged with mutations in key active site residues (Marchand C, et al., Curr. Top. Med. Chem. 9:1016-1037 (2009); Hare S, et al., Mol Pharmacol In Press (2011); Hare S, et al., Proc Natl Acad Sci USA 107(46):20057-20062 (2010)). Importantly, the raltegravir-resistant mutants characterized do not alter the metal binding motif of the enzyme (Metifiot M, et al., Biochemistry 49:3715-3722 (2010)). Indeed, substitution of any of the three metal-binding residues abolishes HIV-1 IN activity, suggesting that metal-binding is essential for HIV-1 IN (Chiu T K & Davies D R, Curr. Top. Med. Chem. 4(9):965-977 (2004)).

The crystal structure of the prototype foamy virus (PFV) integrase bound to its cognate DNA (intasome) has been obtained (Hare S, et al., Nature 464:232-237 (2010)). Structures have also been determined in complex with several inhibitors, including raltegravir. The intasome structures show that these INSTIs have two common features: a) a heteroatom triad to bind the dinuclear metal center, and b) a halogenated benzene ring that serves to displace the 3′ adenine of the bound viral DNA (Hare S, et al., Proc Natl Acad Sci USA 107(46):20057-20062 (2010)). The structure of raltegravir bound to the PFV intasome reveals that both active site Mg²⁺ ions are coordinated by the inhibitor as shown schematically in FIG. 1. Other advanced HIV-1 IN inhibitors, such as elvitegravir, dolutegravir, MK2048, and MK0536 (Hare S, et al., Mol Pharmacol In Press (2011); Hare S, et al., Proc Natl Acad Sci USA 107(46):20057-20062 (2010); Hare S, et al., Nature 464:232-237 (2010)), were also shown to use similar heteroatom triads for binding the dinuclear Mg²⁺ center (FIG. 1). However, the metal-binding atoms in these compounds are not the same, which use different combinations of carbonyl and phenolic oxygen atoms, or even endocyclic pyridyl-nitrogen atoms (Hare S, et al., Proc Natl Acad Sci USA 107(46):20057-20062 (2010)). In addition, the inhibitors do not have identical bond angles between the donor atoms. This indicates that different metal-binding atoms in several different relative orientations can accommodate the HIV-1 IN active site (Marchand C, et al., Curr. Top. Med. Chem. 9:1016-1037 (2009); Hare S, et al., Proc Natl Acad Sci USA 107(46):20057-20062 (2010); Hare S, et al., Nature 464:232-237 (2010)); however, no systemic study that examines these various features within a single chemical scaffold has been reported (Bacchi A, et al., J. Med. Chem. :ASAP contents (2011); Kirschberg T & Parrish J, Curr. Opin. Drug Discov. Dev. 10:460-472 (2007)). The present invention overcomes these and other problems in the art by providing new compounds with HIV integrase inhibiting activity. Further, methods of treatment HIV infection are provided using the compounds of the present invention.

### BRIEF SUMMARY OF THE INVENTION

Provided herein, inter alia, are novel compounds for the inhibition of HIV integrase. The compounds disclosed herein inhibit HIV integrase and are therefore useful for methods of treating HIV infection in a subject in need thereof.

In one aspect, a compound is provided having the formula:

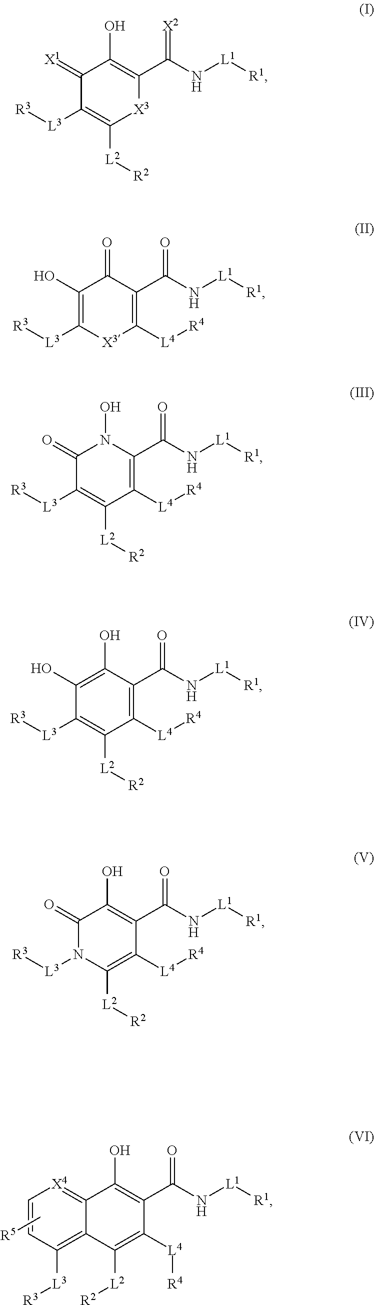

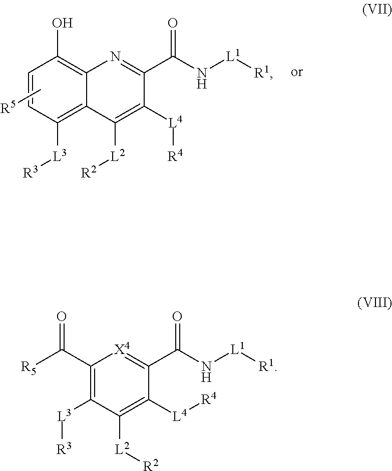

X¹ and X² are, independently ═O or ═S. X³ is —O—, or —N(-L⁴-R⁴)—. X^{3′} is —O—, or —N(-L²-R²)—. X⁴ is —C(OH)═, —N═, or —N⁺(O)═. R¹, R², R³, and R⁴ are, independently, hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁵ is hydrogen, —OR⁶, —NHR⁷, —SO₂NR⁸, —C(O)NR⁹, —C(O)—OR¹⁰, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶, R⁷, R⁸, R⁹, and R¹⁰ are independently hydrogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L¹, L², L³ and L⁴ are independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In another aspect, a pharmaceutical composition is provided. The pharmaceutical composition includes a pharmaceutically acceptable excipient and a compound provided herein including embodiments thereof.

In one aspect, a method of treating an infectious disease in a subject in need thereof is provided. The method includes administering to the subject a therapeutically effective amount of a compound provided herein including embodiments thereof.

In another aspect, a method of inhibiting HIV integrase is provided. The method includes contacting HIV integrase with an effective amount of a compound provided herein including embodiments thereof thereby inhibiting the HIV integrase.

In another aspect, a method of inhibiting HIV integrase in a patient is provided. The method includes administering to the patient a therapeutically effective amount of a compound provided herein including embodiments thereof thereby inhibiting HIV integrase in said patient.

### BRIEF DESCRIPTION OF THE DRAWINGS

FIG. 1. Proposed mode of metal binding for the FDA-approved HIV integrase inhibitor raltegravir (in raised box, left). Structure and strand transfer IC₅₀ values of advanced HIV-1 IN inhibitors, including raltegravir and its abbreviated analog RCD-1 (right). Proposed metal-binding atoms are shown in bold for each inhibitor. Raltegravir and RCD-1 are identical, except that RCD-1 lacks an oxadiazolyl substituent.

FIG. 2. Comparison of the computational docking of RCD-1 in the PFV IN versus the reported crystal structure of raltegravir bound in PFV IN (PDB: 3OYA). The RMSD between the inhibitors is 0.25 Å. Mg_{A} and Mg_{B} are shown as labeled spheres.

FIGS. 3A-3I. MBG numbering system and modes of metal coordination for raltegravir (FIG. 3A) and select RCD compounds (FIGS. 3B-3I). Atoms in bold are part of the heteroatom donor triad, which coordinate to the active site Mg²⁺ ions. Chelate rings with Mg_{A} and Mg_{B} are highlighted. Legend: RCD-1 (FIG. 3A); RCD-2 (FIG. 3B); RCD-3 (FIG. 3C); RCD-5 (FIG. 3D); RCD-6 (FIG. 3E); RCD-12 (FIG. 3F); RCD-13 (FIG. 3G); RCD-14 (FIG. 3H); RCD-15 (FIG. 3I).

FIG. 4. Computational docking results for RCD-12 (top) and RCD-13 (bottom) in the PFV IN active site (PDB: 3OYA). Mg²⁺ ions are shown as spheres and bonding contacts between the inhibitor and metal ions are shown as dashed lines.

FIG. 5. Docked structure of RCD-1 in the active site of PFV-IN (PDB: 3OYA). The inhibitor is shown in sticks, the enzyme as a ribbon, and the Mg²⁺ ions as spheres.

FIG. 6. Docked structure of RCD-2 in the active site of PFV-IN (PDB: 3OYA). The inhibitor is shown in sticks, the enzyme as a ribbon, and the Mg²⁺ ions as spheres.

FIG. 7. Docked structure of RCD-3 in the active site of PFV-IN (PDB: 3OYA). The inhibitor is shown in sticks, the enzyme as a ribbon, and the Mg²⁺ ions as spheres.

FIG. 8. Docked structure of RCD-4 in the active site of PFV-IN (PDB: 3OYA). The inhibitor is shown in sticks, the enzyme as a ribbon, and the Mg²⁺ ions as spheres.

FIG. 9. Docked structure of RCD-5 in the active site of PFV-IN (PDB: 3OYA). The inhibitor is shown in sticks, the enzyme as a ribbon, and the Mg²⁺ ions as spheres.

FIG. 10. Docked structure of RCD-6 in the active site of PFV-IN (PDB: 3OYA). The inhibitor is shown in sticks, the enzyme as a ribbon, and the Mg²⁺ ions as spheres.

FIG. 11. Docked structure of RCD-7 in the active site of PFV-IN (PDB: 3OYA). The inhibitor is shown in sticks, the enzyme as a ribbon, and the Mg²⁺ ions as spheres.

FIG. 12. Docked structure of RCD-8 in the active site of PFV-IN (PDB: 3OYA). The inhibitor is shown in sticks, the enzyme as a ribbon, and the Mg²⁺ ions as spheres.

FIG. 13. Docked structure of RCD-9 in the active site of PFV-IN (PDB: 3OYA). The inhibitor is shown in sticks, the enzyme as a ribbon, and the Mg²⁺ ions as spheres.

FIG. 14. Docked structure of RCD-10 in the active site of PFV-IN (PDB: 3OYA). The inhibitor is shown in sticks, the enzyme as a ribbon, and the Mg²⁺ ions as spheres.

FIG. 15. Docked structure of RCD-11 in the active site of PFV-IN (PDB: 3OYA). The inhibitor is shown in sticks, the enzyme as a ribbon, and the Mg²⁺ ions as spheres.

FIG. 16. Docked structure of RCD-12 in the active site of PFV-IN (PDB: 3OYA). The inhibitor is shown in sticks, the enzyme as a ribbon, and the Mg²⁺ ions as spheres.

FIG. 17. Docked structure of RCD-13 in the active site of PFV-IN (PDB: 3OYA). The inhibitor is shown in sticks, the enzyme as a ribbon, and the Mg²⁺ ions as spheres.

FIG. 18. Docked structure of RCD-14 in the active site of PFV-IN (PDB: 3OYA). The inhibitor is shown in sticks, the enzyme as a ribbon, and the Mg²⁺ ions as spheres.

FIG. 19. Docked structure of RCD-15 in the active site of PFV-IN (PDB: 3OYA). The inhibitor is shown in sticks, the enzyme as a ribbon, and the Mg²⁺ ions as spheres.

FIG. 20. Docked structure of RCD-16 in the active site of PFV-IN (PDB: 3OYA). The inhibitor is shown in sticks, the enzyme as a ribbon, and the Mg²⁺ ions as spheres.

FIG. 21. Docked structure of RCD-17 in the active site of PFV-IN (PDB: 3OYA). The inhibitor is shown in sticks, the enzyme as a ribbon, and the Mg²⁺ ions as spheres.

FIG. 22. Docked structure of RCD-18 in the active site of PFV-IN (PDB: 3OYA). The inhibitor is shown in sticks, the enzyme as a ribbon, and the Mg²⁺ ions as spheres.

FIG. 23. Docked structure of RCD-19 in the active site of PFV-IN (PDB: 3OYA). The inhibitor is shown in sticks, the enzyme as a ribbon, and the Mg²⁺ ions as spheres.

FIG. 24. Docked structure of RCD-5 (top) and RCD-6 (bottom) in the active site of PFV-IN (PDB: 3OYA). From this perspective, the steric clash between the inhibitor methyl group in RCD-6 and Pro 124 is apparent; no such clash exists for RCD-5. The inhibitor is shown in stick (some atoms shown as balls), the enzyme as a ribbon, and the Mg²⁺ ions as spheres.

FIGS. 25A-25B. Representative denaturing sequencing gel (FIG. 25A) and titration curves (FIG. 25B) for RCD compounds. Strand transfer products (labeled ‘STP’), full-length DNA substrate (labeled ‘21’), and 3′-processed products (labeled ‘19’) are noted on the gel. Strand transfer inhibition shows a clear dependence on the MBG.

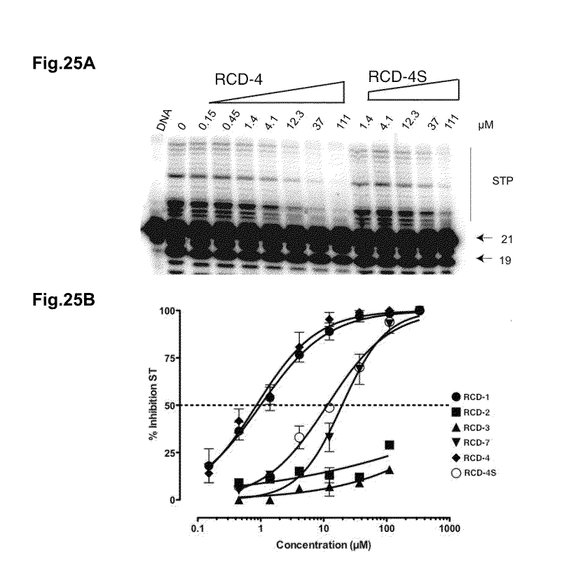

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butyryl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (e.g., from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom selected from N, O, and S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroaryl refers to two rings fused together, wherein one ring has 5 members and the other ring has 6 members, and wherein at least one ring is a heteroaryl ring. Likewise, a 6,6-fused ring heteroaryl refers to two rings fused together, wherein one ring has 6 members and the other ring has 6 members, and wherein at least one ring is a heteroaryl ring. And a 6,5-fused ring heteroaryl refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are as disclosed herein or can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound disclosed herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are disclosed herein or may be selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are referably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl.

Where a moiety is substituted with an R substituent, the group may be referred to as “R-substituted.” Where a moiety is R-substituted, the moiety is substituted with at least one R substituent and each R substituent is optionally different. For example, where a moiety herein is R¹²-substituted or unsubstituted alkyl, a plurality of R¹² substituents may be attached to the alkyl moiety wherein each R¹² substituent is optionally different. Where an R-substituted moiety is substituted with a plurality R substituents, each of the R-substituents may be differentiated herein using a prime symbol (′) such as R′, R″, etc. For example, where a moiety is R¹²-substituted or unsubstituted alkyl, and the moiety is substituted with a plurality of R¹² substituents, the plurality of R¹² substitutents may be differentiated as R¹²′, R¹²″, R¹²′″, etc. In some embodiments, the plurality of R substituents is 3. In some embodiments, the plurality of R substituents is 2.

Description of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The symbol “ [symbol] ” denotes the point of attachment of a chemical moiety to the remainder of a molecule or chemical formula.

As defined herein, the term “inhibition”, “inhibit”, “inhibiting” and the like in reference to a protein-inhibitor (e.g., compound) interaction means negatively affecting (e.g., decreasing) the activity or function of the protein (e.g., decreasing the strand transfer reaction of HIV integrase) relative to the activity or function of the protein in the absence of the inhibitor (e.g., compound). In some embodiments inhibition refers to reduction of a disease or symptoms of disease. In some embodiments, inhibition refers to a reduction in the presence of a disease-related agent (e.g., an infectious agent, infectious agent resistant to one or more anti-HIV integrase inhibitors). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound that inhibits viral survival, growth, or replication, e.g., by binding, partially or totally blocking, decreasing, preventing, delaying, inactivating, desensitizing, or down-regulating enzymatic activity (e.g., strand transfer during viral integration).

The term “effective amount” or “therapeutically effective amount” refers to the amount of an active agent sufficient to induce a desired biological result. That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

A “subject,” “individual,” or “patient,” is used interchangeably herein, which refers to a vertebrate, preferably a mammal, more preferably a human. Mammals include, but are not limited to, murines, simians, humans, farm animals, sport animals, and pets. Tissues, cells and their progeny of a biological entity obtained in vitro or cultured in vitro are also encompassed.

As used herein, the term “infectious disease” refers to a disease or condition related to the presence of an organism (the agent or infectious agent) within or contacting the subject or patient. Examples include a bacterium, fungus, virus, or other microorganism. A “bacterial infectious disease” is an infectious disease wherein the organism is a bacterium. A “viral infectious disease” is an infectious disease wherein the organism is a virus.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine. and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

### II. Compositions

In one aspect, a compound is provided having the formula:

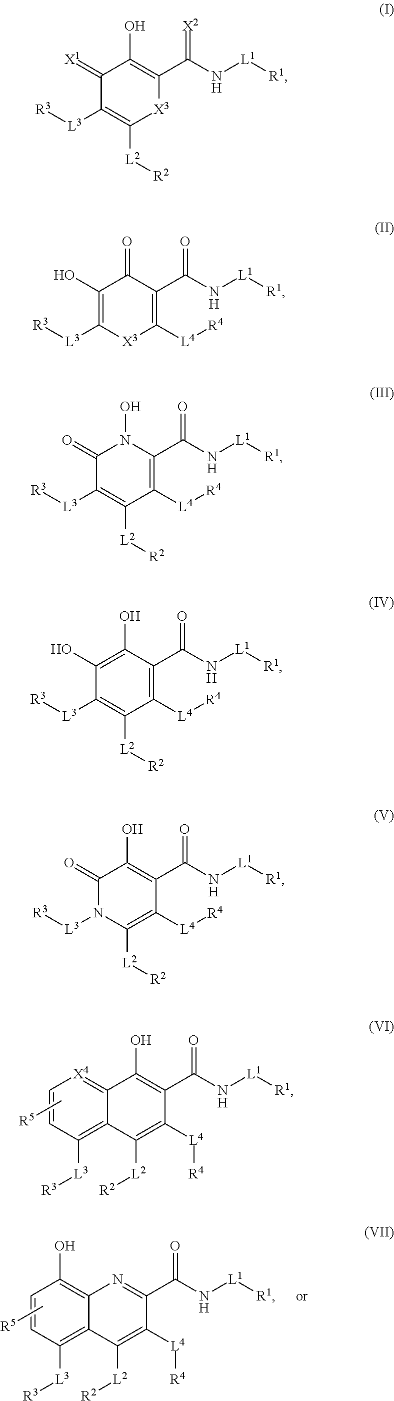

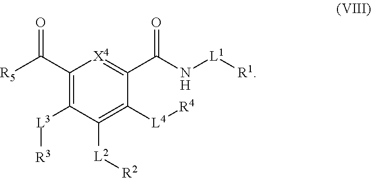

X¹ and X² are, independently ═O or ═S. X³ is —O—, or —N(-L⁴-R⁴)—. X^{3′} is —O—, or —N(-L²-R²)—. X⁴ is —C(OH)═, —N═, or —N⁺(O)═. R¹, R², R³, and R⁴ are, independently, hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁵ is hydrogen, —OR⁶, —NHR⁷, —SO₂NR⁸, —C(O)NR⁹, —C(O)—OR¹⁰, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R⁶, R⁷, R⁸, R⁹, and R¹⁰ are independently hydrogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L¹, L², L³ and L⁴ are independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In some embodiments, the compound has the structure of Formula (I). In other embodiments, the compound has the structure of Formula (II). In other embodiments, the compound has the structure of Formula (III). In some embodiments, the compound has the structure of Formula (IV). In other embodiments, the compound has the structure of Formula (V). In some embodiments, the compound has the structure of Formula (VI). In other embodiments, the compound has the structure of Formula (VII). In other embodiments, the compound has the structure of Formula (VIII).

R¹, R², R³, and R⁴ may be the same or different and may each independently be hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹, R², R³, and R⁴ may be the same or different and may each independently be hydrogen, halogen, —CF₃, —CN, —CCl₃, —COOH, —CH₂COOH, —CONH₂, —OH, —SH, —NO₂, —NH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R¹, R², R³, and R⁴ are, independently, hydrogen, substituted or unsubstituted C₁-C₂₀ (e.g., C₁-C₆) alkyl, substituted or unsubstituted 2 to 20 membered (e.g., 2 to 6 membered) heteroalkyl, substituted or unsubstituted C₃-C₈ (e.g., C₅-C₇) cykloalkyl, substituted or unsubstituted 3 to 8 membered (e.g., 3 to 6 membered) heterocycloalkyl, substituted or unsubstituted C₅-C₁₀ (e.g., C₅-C₆) aryl, or substituted or unsubstituted 5 to 10 membered (e.g., 5 to 6 membered) heteroaryl.

In some embodiments, R¹ is hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. In some embodiments, R¹ is hydrogen, halogen, —CF₃, —CN, —CCl₃, —COOH, —CH₂COOH, —CONH₂, —OH, —SH, —NO₂, —NH₂, R¹¹-substituted or unsubstituted alkyl, R¹¹-substituted or unsubstituted heteroalkyl, R¹¹-substituted or unsubstituted cycloalkyl, R¹¹-substituted or unsubstituted heterocycloalkyl, R¹¹-substituted or unsubstituted aryl, or R¹¹-substituted or unsubstituted heteroaryl. R¹ may be hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹¹-substituted or unsubstituted C₁-C₂₀ (e.g., C₁-C₆) alkyl, R¹¹-substituted or unsubstituted 2 to 20 membered (e.g., 2 to 6 membered) heteroalkyl, R¹¹-substituted or unsubstituted C₃-C₈ (e.g., C₅-C₇) cykloalkyl, R¹¹-substituted or unsubstituted 3 to 8 membered (e.g., 3 to 6 membered) heterocycloalkyl, R¹¹-substituted or unsubstituted C₅-C₁₀ (e.g., C₅-C₆) aryl, or R¹¹-substituted or unsubstituted 5 to 10 membered (e.g., 5 to 6 membered) heteroaryl.

R¹¹ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R¹¹ is ═O, R¹ is not aryl or heteroaryl. In some embodiments, R¹¹ is R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl. R¹¹ may be R¹²-substituted or unsubstituted C₁-C₂₀ (e.g., C₁-C₆) alkyl, R¹²-substituted or unsubstituted 2 to 20 membered (e.g., 2 to 6 membered) heteroalkyl, R¹²-substituted or unsubstituted C₃-C₈ (e.g., C₅-C₇) cykloalkyl, R¹²-substituted or unsubstituted 3 to 8 membered (e.g., 3 to 6 membered) heterocycloalkyl, R¹²-substituted or unsubstituted C₅-C₁₀ (e.g., C₅-C₆) aryl, or R¹²-substituted or unsubstituted 5 to 10 membered (e.g., 5 to 6 membered) heteroaryl.

R¹² is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R¹² is ═O, R¹¹ is not aryl or heteroaryl. In some embodiments, R¹² is R¹³-substituted or unsubstituted alkyl, R¹³-substituted or unsubstituted heteroalkyl, R¹³-substituted or unsubstituted cycloalkyl, R¹³-substituted or unsubstituted heterocycloalkyl, R¹³-substituted or unsubstituted aryl, or R¹³-substituted or unsubstituted heteroaryl. R¹² may be R¹³-substituted or unsubstituted C₁-C₂₀ (e.g., C₁-C₆) alkyl, R¹³-substituted or unsubstituted 2 to 20 membered (e.g., 2 to 6 membered) heteroalkyl, R¹³-substituted or unsubstituted C₃-C₈ (e.g., C₅-C₇) cykloalkyl, R¹³-substituted or unsubstituted 3 to 8 membered (e.g., 3 to 6 membered) heterocycloalkyl, R¹³-substituted or unsubstituted C₅-C₁₀ (e.g., C₅-C₆) aryl, or R¹³-substituted or unsubstituted 5 to 10 membered (e.g., 5 to 6 membered) heteroaryl.

R¹³ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R¹³ is ═O, R¹² is not aryl or heteroaryl. In some embodiments, R¹³ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In some embodiments, R¹ is substituted (e.g., R¹¹-substituted) or unsubstituted C₅-C₁₀ aryl. R¹ may be substituted (e.g., R¹¹-substituted) or unsubstituted C₅-C₆ aryl. In some further embodiments, R¹ is substituted (e.g., R¹¹-substituted) or unsubstituted phenyl. In some further embodiments, R¹ is halophenyl. A “halophenyl” as provided herein refers to a phenyl substituted with at least one halogen (e.g., one halogen).

R¹ may be R¹¹-substituted aryl and R¹¹ may be halogen. In some embodiments, R¹ is R¹¹-substituted C₅-C₁₀ (e.g., C₅-C₆) aryl and R¹¹ is halogen. Thus, in some embodiments, R¹ is R¹¹-substituted C₆ aryl and R¹¹ is halogen. In some embodiments, R¹ is R¹¹-substituted phenyl and R¹¹ is halogen. In some further embodiments, R¹¹ is fluorine. Thus, in some embodiments, R¹ is halophenyl. In some embodiments, there is only one R¹¹. In some further embodiments, R¹¹ is halogen. In some further embodiments, R¹¹ is fluorine.

In some embodiments, -L¹-R¹ has the structure of Formula

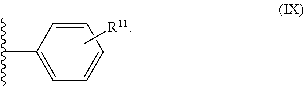

In some further embodiments, R¹¹ is halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, R¹¹ is R¹²-substituted or unsubstituted alkyl, R¹²-substituted or unsubstituted heteroalkyl, R¹²-substituted or unsubstituted cycloalkyl, R¹²-substituted or unsubstituted heterocycloalkyl, R¹²-substituted or unsubstituted aryl, or R¹²-substituted or unsubstituted heteroaryl. R¹¹ may be R¹²-substituted or unsubstituted C₁-C₂₀ (e.g., C₁-C₆) alkyl, R¹²-substituted or unsubstituted 2 to 20 membered (e.g., 2 to 6 membered) heteroalkyl, R¹²-substituted or unsubstituted C₃-C₈ (e.g., C₅-C₇) cykloalkyl, R¹²-substituted or unsubstituted 3 to 8 membered (e.g., 3 to 6 membered) heterocycloalkyl, R¹²-substituted or unsubstituted C₅-C₁₀ (e.g., C₅-C₆) aryl, or R¹²-substituted or unsubstituted 5 to 10 membered (e.g., 5 to 6 membered) heteroaryl. In some further embodiments, R¹¹ is halogen. In some further embodiments, R¹¹ is fluorine. In some further embodiments, there is only one R¹¹.

In some embodiments, -L¹-R¹ has the structure of Formula

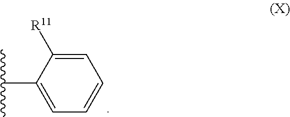

In some further embodiments, R¹¹ is halogen. In some further embodiments, R¹¹ is fluorine. In some other embodiments, -L¹-R¹ has the Formula

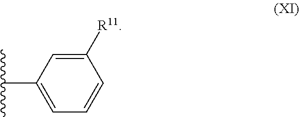

In some further embodiments, R¹¹ is halogen. In some further embodiments, R¹¹ is fluorine. In some embodiments, -L¹-R¹ has the structure of Formula

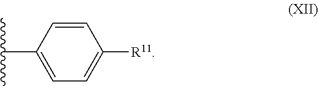

In some further embodiments, R¹¹ is halogen. In some further embodiments, R¹¹ is fluorine.

In some embodiments, R² is hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁴-substituted or unsubstituted alkyl, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or R¹⁴-substituted or unsubstituted heteroaryl. In some embodiments, R² is hydrogen, halogen, —CF₃, —CN, —CCl₃, —COOH, —CH₂COOH, —CONH₂, —OH, —SH, —NO₂, —NH₂, R¹⁴-substituted or unsubstituted alkyl, R¹⁴-substituted or unsubstituted heteroalkyl, R¹⁴-substituted or unsubstituted cycloalkyl, R¹⁴-substituted or unsubstituted heterocycloalkyl, R¹⁴-substituted or unsubstituted aryl, or R¹⁴-substituted or unsubstituted heteroaryl. R² may be hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁴-substituted or unsubstituted C₁-C₂₀ (e.g., C₁-C₆) alkyl, R¹⁴-substituted or unsubstituted 2 to 20 membered (e.g., 2 to 6 membered) heteroalkyl, R¹⁴-substituted or unsubstituted C₃-C₈ (e.g., C₅-C₇) cykloalkyl, R¹⁴-substituted or unsubstituted 3 to 8 membered (e.g., 3 to 6 membered) heterocycloalkyl, R¹⁴-substituted or unsubstituted C₅-C₁₀ (e.g., C₅-C₆) aryl, or R¹⁴-substituted or unsubstituted 5 to 10 membered (e.g., 5 to 6 membered) heteroaryl.

R¹⁴ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R¹⁴ is ═O, R² is not aryl or heteroaryl. In some embodiments, R¹⁴ is R¹⁵-substituted or unsubstituted alkyl, R¹⁵-substituted or unsubstituted heteroalkyl, R¹⁵-substituted or unsubstituted cycloalkyl, R¹⁵-substituted or unsubstituted heterocycloalkyl, R¹⁵-substituted or unsubstituted aryl, or R¹⁵-substituted or unsubstituted heteroaryl. R¹⁴ may be R¹⁵-substituted or unsubstituted C₁-C₂₀ (e.g., C₁-C₆) alkyl, R¹⁵-substituted or unsubstituted 2 to 20 membered (e.g., 2 to 6 membered) heteroalkyl, R¹⁵-substituted or unsubstituted C₃-C₈ (e.g., C₅-C₇) cykloalkyl, R¹⁵-substituted or unsubstituted 3 to 8 membered (e.g., 3 to 6 membered) heterocycloalkyl, R¹⁵-substituted or unsubstituted C₅-C₁₀ (e.g., C₅-C₆) aryl, or R¹⁵-substituted or unsubstituted 5 to 10 membered (e.g., 5 to 6 membered) heteroaryl.

R¹⁵ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R¹⁵ is ═O, R¹⁴ is not aryl or heteroaryl. In some embodiments, R¹⁵ is R¹⁶-substituted or unsubstituted alkyl, R¹⁶-substituted or unsubstituted heteroalkyl, R¹⁶-substituted or unsubstituted cycloalkyl, R¹⁶-substituted or unsubstituted heterocycloalkyl, R¹⁶-substituted or unsubstituted aryl, or R¹⁶-substituted or unsubstituted heteroaryl. R¹⁵ may be R¹⁶-substituted or unsubstituted C₁-C₂₀ (e.g., C₁-C₆) alkyl, R¹⁶-substituted or unsubstituted 2 to 20 membered (e.g., 2 to 6 membered) heteroalkyl, R¹⁶-substituted or unsubstituted C₃-C₈ (e.g., C₅-C₇) cykloalkyl, R¹⁶-substituted or unsubstituted 3 to 8 membered (e.g., 3 to 6 membered) heterocycloalkyl, R¹⁶-substituted or unsubstituted C₅-C₁₀ (e.g., C₅-C₆) aryl, or R¹⁶-substituted or unsubstituted 5 to 10 membered (e.g., 5 to 6 membered) heteroaryl.

R¹⁶ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R¹⁶ is ═O, R¹⁵ is not aryl or heteroaryl. In some embodiments, R¹⁶ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In some embodiments, R² is substituted (e.g., R¹⁴-substituted) or unsubstituted 5 to 10 membered (e.g., 5 to 6 membered) heteroaryl. In some embodiments, R² is substituted (e.g., R¹⁴-substituted) 5 to 10 membered (e.g., 5 to 6 membered) heteroaryl. In other embodiments, R² is substituted (e.g., R¹⁴-substituted) 5 to 6 membered (e.g., 5 membered) heteroaryl. In other embodiments, R² is substituted (e.g., R¹⁴-substituted) oxadiazolyl.

R² may be R¹⁴-substituted or unsubstituted 5 to 10 membered (e.g., 5 to 6 membered) heteroaryl. In some embodiments, R² is R¹⁴-substituted 5 to 10 membered (e.g., 5 to 6 membered) heteroaryl. In other embodiments, R² is R¹⁴-substituted 5 to 6 membered (e.g., 5 membered) heteroaryl. Thus, in some embodiments, R² is R¹⁴-substituted oxadiazolyl. R¹⁴ may be substituted or unsubstituted alkyl. Thus, in some further embodiments, R¹⁴ is substituted or unsubstituted C₁-C₂₀ (e.g., C₁-C₁₂) alkyl. In some further embodiments, R¹⁴ is substituted or unsubstituted C₁-C₁₀ (e.g., C₁-C₆) alkyl. In further embodiments, R¹⁴ is substituted or unsubstituted C₁-C₄ (e.g., C₁-C₂) alkyl. In some further embodiments, R¹⁴ is unsubstituted C₁-C₄ (e.g., C₁-C₂) alkyl. Thus, R¹⁴ may be ethyl or methyl. In some further embodiments, R¹⁴ is methyl.

L² may be substituted or unsubstituted alkylene or substituted or unsubstituted heteroalkylene. In some embodiments, L² is-substituted or unsubstituted C₁-C₂₀ (e.g., C₁-C₈) alkylene, or substituted or unsubstituted 2 to 20 membered (e.g., 2 to 8 membered) heteroalkylene. In some embodiments, L² is substituted or unsubstituted 2 to 20 membered (e.g., 2 to 8 membered) heteroalkylene. In other embodiments, L² is substituted or unsubstituted 2 to 10 membered (e.g., 2 to 6 membered) heteroalkylene. In some embodiments, L² is substituted or unsubstituted 2 to 6 membered heteroalkylene. In other embodiments, L² is unsubstituted 2 to 6 membered heteroalkylene. In some embodiments, L² is unsubstituted 4 membered heteroalkylene.

The compound provided herein may include -L²-R² having the structure of formula

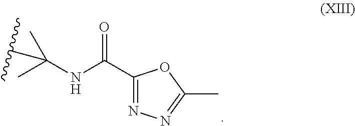 [SMILES: *C(C)(C)NC(=O)c1nnc(C)o1]

In some embodiments, -L²-R² has the structure of Formula

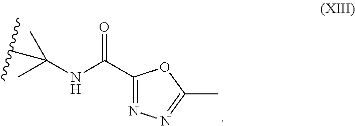 [SMILES: *C(C)(C)NC(=O)c1nnc(C)o1]

In some embodiments, L² has the structure of Formula

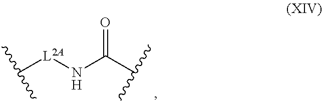

wherein the point of attachment on the right side of L² connects to R² and the point of attachment on the left side of L² binds to the remainder of the molecule. L^{2A} is R⁴⁴-substituted or unsubstituted alkylene. In some embodiments, L^{2A} is R⁴⁴-substituted or unsubstituted C₁-C₂₀ (e.g., C₁-C₆) alkylene. In some embodiments, L^{2A} is R⁴⁴-substituted C₁-C₂₀ (e.g., C₁-C₆) alkylene. In other embodiments, L^{2A} is R⁴⁴-substituted C₁-C₄ (e.g., ethylene or methylene) alkylene. In some embodiments, L^{2A} is R⁴⁴-substituted methylene. In some embodiments, L^{2A} is R⁴⁴-substituted C₁-C₄ (e.g., ethylene or methylene) alkylene and R⁴⁴ is unsubstituted C₁-C₂₀ (e.g., C₁-C₆) alkyl. In some embodiments, L^{2A} is R⁴⁴-substituted methylene and R⁴⁴ is unsubstituted C₁-C₄ (e.g., ethyl or methyl) alkyl. R⁴⁴ is as defined below. In some embodiments, L^{2A} is R⁴⁴-substituted methylene and R⁴⁴ is methyl.

In some embodiments, -L²-R² has the structure of Formula

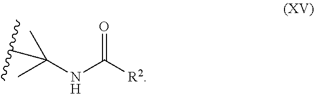

R² is R¹⁴-substituted or unsubstituted heteroaryl. In some embodiments, R² is R¹⁴-substituted or unsubstituted 5 to 10 membered (e.g., 5 to 6 membered) heteroaryl. In other embodiments, R² is R¹⁴-substituted 5 to 10 membered (e.g., 5 to 6 membered) heteroaryl. In other embodiments, R² is R¹⁴-substituted 5 membered heteroaryl. In some embodiments, R² is R¹⁴-substituted oxadiazolyl. R¹⁴ may be substituted or unsubstituted alkyl. Thus, in some further embodiments, R¹⁴ is substituted or unsubstituted C₁-C₂₀ (e.g., C₁-C₁₂) alkyl. In some further embodiments, R¹⁴ is substituted or unsubstituted C₁-C₁₀ (e.g., C₁-C₆) alkyl. In further embodiments, R¹⁴ is substituted or unsubstituted C₁-C₄ (e.g., C₁-C₂) alkyl. In some further embodiments, R¹⁴ is unsubstituted C₁-C₄ (e.g., C₁-C₂) alkyl. Thus, R¹⁴ may be ethyl or methyl. In some further embodiments, R¹⁴ is methyl.

In some embodiments, R³ is hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁷-substituted or unsubstituted alkyl, R¹⁷-substituted or unsubstituted heteroalkyl, R¹⁷-substituted or unsubstituted cycloalkyl, R¹⁷-substituted or unsubstituted heterocycloalkyl, R¹⁷-substituted or unsubstituted aryl, or R¹⁷-substituted or unsubstituted heteroaryl. In some embodiments, R³ is hydrogen, halogen, —CF₃, —CN, —CCl₃, —COOH, —CH₂COOH, —CONH₂, —OH, —SH, —NO₂, —NH₂, R¹⁷-substituted or unsubstituted alkyl, R¹⁷-substituted or unsubstituted heteroalkyl, R¹⁷-substituted or unsubstituted cycloalkyl, R¹⁷-substituted or unsubstituted heterocycloalkyl, R¹⁷-substituted or unsubstituted aryl, or R¹⁷-substituted or unsubstituted heteroaryl. R³ may be hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R¹⁷-substituted or unsubstituted C₁-C₂₀ (e.g., C₁-C₆) alkyl, R¹⁷-substituted or unsubstituted 2 to 20 membered (e.g., 2 to 6 membered) heteroalkyl, R¹⁷-substituted or unsubstituted C₃-C₈ (e.g., C₅-C₇) cykloalkyl, R¹⁷-substituted or unsubstituted 3 to 8 membered (e.g., 3 to 6 membered) heterocycloalkyl, R¹⁷-substituted or unsubstituted C₅-C₁₀ (e.g., C₅-C₆) aryl, or R¹⁷-substituted or unsubstituted 5 to 10 membered (e.g., 5 to 6 membered) heteroaryl.

R¹⁷ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R¹⁷ is ═O, R³ is not aryl or heteroaryl. In some embodiments, R¹⁷ is R¹⁸-substituted or unsubstituted alkyl, R¹⁸-substituted or unsubstituted heteroalkyl, R¹⁸-substituted or unsubstituted cycloalkyl, R¹⁸-substituted or unsubstituted heterocycloalkyl, R¹⁸-substituted or unsubstituted aryl, or R¹⁸-substituted or unsubstituted heteroaryl. R¹⁷ may be R¹⁸-substituted or unsubstituted C₁-C₂₀ (e.g., C₁-C₆) alkyl, R¹⁸-substituted or unsubstituted 2 to 20 membered (e.g., 2 to 6 membered) heteroalkyl, R¹⁸-substituted or unsubstituted C₃-C₈ (e.g., C₅-C₇) cykloalkyl, R¹⁸-substituted or unsubstituted 3 to 8 membered (e.g., 3 to 6 membered) heterocycloalkyl, R¹⁸-substituted or unsubstituted C₅-C₁₀ (e.g., C₅-C₆) aryl, or R¹⁸-substituted or unsubstituted 5 to 10 membered (e.g., 5 to 6 membered) heteroaryl.

R¹⁸ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R¹⁸ is ═O, R¹⁷ is not aryl or heteroaryl. In some embodiments, R¹⁸ is R¹⁹-substituted or unsubstituted alkyl, R¹⁹-substituted or unsubstituted heteroalkyl, R¹⁹-substituted or unsubstituted cycloalkyl, R¹⁹-substituted or unsubstituted heterocycloalkyl, R¹⁹-substituted or unsubstituted aryl, or R¹⁹-substituted or unsubstituted heteroaryl. R¹⁸ may be R¹⁹-substituted or unsubstituted C₁-C₂₀ (e.g., C₁-C₆) alkyl, R¹⁹-substituted or unsubstituted 2 to 20 membered (e.g., 2 to 6 membered) heteroalkyl, R¹⁹-substituted or unsubstituted C₃-C₈ (e.g., C₅-C₇) cykloalkyl, R¹⁹-substituted or unsubstituted 3 to 8 membered (e.g., 3 to 6 membered) heterocycloalkyl, R¹⁹-substituted or unsubstituted C₅-C₁₀ (e.g., C₅-C₆) aryl, or R¹⁹-substituted or unsubstituted 5 to 10 membered (e.g., 5 to 6 membered) heteroaryl.

R¹⁹ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R¹⁸ is ═O, R¹⁹ is not aryl or heteroaryl. In some embodiments, R¹⁹ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

L³ may be a bond when R³ is hydrogen. For the compounds provided herein including embodiments thereof, R³ may be hydrogen and L³ may be a bond. Thus, in some embodiments, R³ is hydrogen and L³ is a bond.

In some embodiments, R⁴ is hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R²⁰-substituted or unsubstituted alkyl, R²⁰-substituted or unsubstituted heteroalkyl, R²⁰-substituted or unsubstituted cycloalkyl, R²⁰-substituted or unsubstituted heterocycloalkyl, R²⁰-substituted or unsubstituted aryl, or R²⁰-substituted or unsubstituted heteroaryl. In some embodiments, R⁴ is hydrogen, halogen, —CF₃, —CN, —CCl₃, —COOH, —CH₂COOH, —CONH₂, —OH, —SH, —NO₂, —NH₂, R²⁰-substituted or unsubstituted alkyl, R²⁰-substituted or unsubstituted heteroalkyl, R²⁰-substituted or unsubstituted cycloalkyl, R²⁰-substituted or unsubstituted heterocycloalkyl, R²⁰-substituted or unsubstituted aryl, or R²⁰-substituted or unsubstituted heteroaryl. R⁴ may be hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R²⁰-substituted or unsubstituted C₁-C₂₀ (e.g., C₁-C₆) alkyl, R²⁰-substituted or unsubstituted 2 to 20 membered (e.g., 2 to 6 membered) heteroalkyl, R²⁰-substituted or unsubstituted C₃-C₈ (e.g., C₅-C₇) cykloalkyl, R²⁰-substituted or unsubstituted 3 to 8 membered (e.g., 3 to 6 membered) heterocycloalkyl, R²⁰-substituted or unsubstituted C₅-C₁₀ (e.g., C₅-C₆) aryl, or R²⁰-substituted or unsubstituted 5 to 10 membered (e.g., 5 to 6 membered) heteroaryl.

R²⁰ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R²⁰ is ═O, R⁴ is not aryl or heteroaryl. In some embodiments, R²⁰ is R²¹-substituted or unsubstituted alkyl, R²¹-substituted or unsubstituted heteroalkyl, R²¹-substituted or unsubstituted cycloalkyl, R²¹-substituted or unsubstituted heterocycloalkyl, R²¹-substituted or unsubstituted aryl, or R²¹-substituted or unsubstituted heteroaryl. R²⁰ may be R²¹-substituted or unsubstituted C₁-C₂₀ (e.g., C₁-C₆) alkyl, R²¹-substituted or unsubstituted 2 to 20 membered (e.g., 2 to 6 membered) heteroalkyl, R²¹-substituted or unsubstituted C₃-C₈ (e.g., C₅-C₇) cykloalkyl, R²¹-substituted or unsubstituted 3 to 8 membered (e.g., 3 to 6 membered) heterocycloalkyl, R²¹-substituted or unsubstituted C₅-C₁₀ (e.g., C₅-C₆) aryl, or R²¹-substituted or unsubstituted 5 to 10 membered (e.g., 5 to 6 membered) heteroaryl.

R²¹ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R²¹ is ═O, R²⁰ is not aryl or heteroaryl. In some embodiments, R²¹ is R²²-substituted or unsubstituted alkyl, R²²-substituted or unsubstituted heteroalkyl, R²²-substituted or unsubstituted cycloalkyl, R²²-substituted or unsubstituted heterocycloalkyl, R²²-substituted or unsubstituted aryl, or R²²-substituted or unsubstituted heteroaryl. R²¹ may be R²²-substituted or unsubstituted C₁-C₂₀ (e.g., C₁-C₆) alkyl, R²²-substituted or unsubstituted 2 to 20 membered (e.g., 2 to 6 membered) heteroalkyl, R²²-substituted or unsubstituted C₃-C₈ (e.g., C₅-C₇) cykloalkyl, R²²-substituted or unsubstituted 3 to 8 membered (e.g., 3 to 6 membered) heterocycloalkyl, R²²-substituted or unsubstituted C₅-C₁₀ (e.g., C₅-C₆) aryl, or R²²-substituted or unsubstituted 5 to 10 membered (e.g., 5 to 6 membered) heteroaryl.

R²² is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R²² is ═O, R²¹ is not aryl or heteroaryl. In some embodiments, R²² is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

L⁴ may be a bond when R⁴ is hydrogen. For the compounds provided herein including embodiments thereof, R⁴ may be hydrogen and L⁴ may be a bond. Thus, in some embodiments, R⁴ is hydrogen and L⁴ is a bond.

R², R³, and R⁴ may be independently substituted or unsubstituted C₁-C₂₀ (e.g., C₁-C₁₀) alkyl or hydrogen. In some embodiments, R², R³, and R⁴ are, independently substituted or unsubstituted C₁-C₁₀ (e.g., C₁-C₆) alkyl or hydrogen. Thus, R², R³, and R⁴ may be independently substituted or unsubstituted C₁-C₄ (e.g., C₁-C₂) alkyl or hydrogen. In some embodiments, R², R³, and R⁴ are, independently unsubstituted C₁-C₄ (e.g., C₁-C₂) alkyl or hydrogen. In other embodiments, R², R³, and R⁴ are, independently methyl, ethyl or hydrogen. In other embodiments, R², R³, and R⁴ are, independently hydrogen.

R⁵ may be hydrogen, —OR⁶, —NHR⁷, —SO₂NR⁸, —C(O)NR⁹, —C(O)—OR¹⁰, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R⁵ is —OR⁶, —NHR⁷, —SO₂NR⁸, —C(O)NR⁹, —C(O)—OR¹⁰, hydrogen, halogen, R²³-substituted or unsubstituted alkyl, R²³-substituted or unsubstituted heteroalkyl, R²³-substituted or unsubstituted cycloalkyl, R²³-substituted or unsubstituted heterocycloalkyl, R²³-substituted or unsubstituted aryl, or R²³-substituted or unsubstituted heteroaryl. R⁵ may be hydrogen, halogen, —OR⁶, —NHR⁷, —SO₂NR⁸, —C(O)NR⁹, —C(O)—OR¹⁰, R²³-substituted or unsubstituted C₁-C₂₀ (e.g., C₁-C₆) alkyl, R²³-substituted or unsubstituted 2 to 20 membered (e.g., 2 to 6 membered) heteroalkyl, R²³-substituted or unsubstituted C₃-C₈ (e.g., C₅-C₇) cykloalkyl, R²³-substituted or unsubstituted 3 to 8 membered (e.g., 3 to 6 membered) heterocycloalkyl, R²³-substituted or unsubstituted C₅-C₁₀ (e.g., C₅-C₆) aryl, or R²³-substituted or unsubstituted 5 to 10 membered (e.g., 5 to 6 membered) heteroaryl.

R²³ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R²³ is ═O, R⁵ is not aryl or heteroaryl. In some embodiments, R²³ is R²⁴-substituted or unsubstituted alkyl, R²⁴-substituted or unsubstituted heteroalkyl, R²⁴-substituted or unsubstituted cycloalkyl, R²⁴-substituted or unsubstituted heterocycloalkyl, R²⁴-substituted or unsubstituted aryl, or R²⁴-substituted or unsubstituted heteroaryl. R²³ may be R²⁴-substituted or unsubstituted C₁-C₂₀ (e.g., C₁-C₆) alkyl, R²⁴-substituted or unsubstituted 2 to 20 membered (e.g., 2 to 6 membered) heteroalkyl, R²⁴-substituted or unsubstituted C₃-C₈ (e.g., C₅-C₇) cykloalkyl, R²⁴-substituted or unsubstituted 3 to 8 membered (e.g., 3 to 6 membered) heterocycloalkyl, R²⁴-substituted or unsubstituted C₅-C₁₀ (e.g., C₅-C₆) aryl, or R²⁴-substituted or unsubstituted 5 to 10 membered (e.g., 5 to 6 membered) heteroaryl.

R²⁴ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R²⁴ is ═O, R²³ is not aryl or heteroaryl. In some embodiments, R²⁴ is R²⁵-substituted or unsubstituted alkyl, R²⁵-substituted or unsubstituted heteroalkyl, R²⁵-substituted or unsubstituted cycloalkyl, R²⁵-substituted or unsubstituted heterocycloalkyl, R²⁵-substituted or unsubstituted aryl, or R²⁵-substituted or unsubstituted heteroaryl. R²⁴ may be R²⁵-substituted or unsubstituted C₁-C₂₀ (e.g., C₁-C₆) alkyl, R²⁵-substituted or unsubstituted 2 to 20 membered (e.g., 2 to 6 membered) heteroalkyl, R²⁵-substituted or unsubstituted C₃-C₈ (e.g., C₅-C₇) cykloalkyl, R²⁵-substituted or unsubstituted 3 to 8 membered (e.g., 3 to 6 membered) heterocycloalkyl, R²⁵-substituted or unsubstituted C₅-C₁₀ (e.g., C₅-C₆) aryl, or R²⁵-substituted or unsubstituted 5 to 10 membered (e.g., 5 to 6 membered) heteroaryl.

R²⁵ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R²⁵ is ═O, R²⁴ is not aryl or heteroaryl. In some embodiments, R²⁵ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In some embodiments, where the compound is the compound of Formula (VIII), R⁵ is not —SO₂NR⁸, —C(O)NR⁹, or —C(O)—OR¹⁰.

In some embodiments, R⁶ is hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R²⁶-substituted or unsubstituted alkyl, R²⁶-substituted or unsubstituted heteroalkyl, R²⁶-substituted or unsubstituted cycloalkyl, R²⁶-substituted or unsubstituted heterocycloalkyl, R²⁶-substituted or unsubstituted aryl, or R²⁶-substituted or unsubstituted heteroaryl. R⁶ may be hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R²⁶-substituted or unsubstituted C₁-C₂₀ (e.g., C₁-C₆) alkyl, R²⁶-substituted or unsubstituted 2 to 20 membered (e.g., 2 to 6 membered) heteroalkyl, R²⁶-substituted or unsubstituted C₃-C₈ (e.g., C₅-C₇) cykloalkyl, R²⁶-substituted or unsubstituted 3 to 8 membered (e.g., 3 to 6 membered) heterocycloalkyl, R²⁶-substituted or unsubstituted C₅-C₁₀ (e.g., C₅-C₆) aryl, or R²⁶-substituted or unsubstituted 5 to 10 membered (e.g., 5 to 6 membered) heteroaryl.

R²⁶ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R²⁶ is ═O, R⁶ is not aryl or heteroaryl. In some embodiments, R²⁶ is R²⁷-substituted or unsubstituted alkyl, R²⁷-substituted or unsubstituted heteroalkyl, R²⁷-substituted or unsubstituted cycloalkyl, R²⁷-substituted or unsubstituted heterocycloalkyl, R²⁷-substituted or unsubstituted aryl, or R²⁷-substituted or unsubstituted heteroaryl. R²⁶ may be R²⁷-substituted or unsubstituted C₁-C₂₀ (e.g., C₁-C₆) alkyl, R²⁷-substituted or unsubstituted 2 to 20 membered (e.g., 2 to 6 membered) heteroalkyl, R²⁷-substituted or unsubstituted C₃-C₈ (e.g., C₅-C₇) cykloalkyl, R²⁷-substituted or unsubstituted 3 to 8 membered (e.g., 3 to 6 membered) heterocycloalkyl, R²⁷-substituted or unsubstituted C₅-C₁₀ (e.g., C₅-C₆) aryl, or R²⁷-substituted or unsubstituted 5 to 10 membered (e.g., 5 to 6 membered) heteroaryl.

R²⁷ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R²⁷ is ═O, where R²⁶ is not aryl or heteroaryl. In some embodiments, R²⁷ is R²⁸-substituted or unsubstituted alkyl, R²⁸-substituted or unsubstituted heteroalkyl, R²⁸-substituted or unsubstituted cycloalkyl, R²⁸-substituted or unsubstituted heterocycloalkyl, R²⁸-substituted or unsubstituted aryl, or R²⁸-substituted or unsubstituted heteroaryl. R²⁷ may be R²⁸-substituted or unsubstituted C₁-C₂₀ (e.g., C₁-C₆) alkyl, R²⁸-substituted or unsubstituted 2 to 20 membered (e.g., 2 to 6 membered) heteroalkyl, R²⁸-substituted or unsubstituted C₃-C₈ (e.g., C₅-C₇) cykloalkyl, R²⁸-substituted or unsubstituted 3 to 8 membered (e.g., 3 to 6 membered) heterocycloalkyl, R²⁸-substituted or unsubstituted C₅-C₁₀ (e.g., C₅-C₆) aryl, or R²⁸-substituted or unsubstituted 5 to 10 membered (e.g., 5 to 6 membered) heteroaryl.

R²⁸ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R²⁸ is ═O, R²⁷ is not aryl or heteroaryl. In some embodiments, R²⁸ is unsubstituted C₁-C₂₀ (e.g., C₁-C₆) alkyl, unsubstituted 2 to 20 membered (e.g., 2 to 6 membered) heteroalkyl, unsubstituted

C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In some embodiments, R⁵ is —OR⁶ or —NHR⁷. R⁶ may be substituted or unsubstituted C₁-C₂₀ (e.g., C₁-C₁₀) alkyl or hydrogen. In some embodiments, R⁶ is substituted or unsubstituted C₁-C₁₀ (e.g., C₁-C₆) alkyl or hydrogen. In other embodiments, R⁶ is substituted or unsubstituted C₁-C₄ (e.g., C₁-C₂) alkyl or hydrogen. In some embodiments, R⁶ is unsubstituted C₁-C₄ (e.g., C₁-C₂) alkyl or hydrogen. In other embodiments, R⁶ is methyl or hydrogen. In other embodiments, R⁶ is hydrogen.

In some embodiments, R⁷ is hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R²⁹-substituted or unsubstituted alkyl, R²⁹-substituted or unsubstituted heteroalkyl, R²⁹-substituted or unsubstituted cycloalkyl, R²⁹-substituted or unsubstituted heterocycloalkyl, R²⁹-substituted or unsubstituted aryl, or R²⁹-substituted or unsubstituted heteroaryl. R⁷ may be hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R²⁹-substituted or unsubstituted C₁-C₂₀ (e.g., C₁-C₆) alkyl, R²⁹-substituted or unsubstituted 2 to 20 membered (e.g., 2 to 6 membered) heteroalkyl, R²⁹-substituted or unsubstituted C₃-C₈ (e.g., C₅-C₇) cykloalkyl, R²⁹-substituted or unsubstituted 3 to 8 membered (e.g., 3 to 6 membered) heterocycloalkyl, R²⁹-substituted or unsubstituted C₅-C₁₀ (e.g., C₅-C₆) aryl, or R²⁹-substituted or unsubstituted 5 to 10 membered (e.g., 5 to 6 membered) heteroaryl.

R²⁹ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R²⁹ is ═O, R⁷ is not aryl or heteroaryl. In some embodiments, R²⁹ is R³⁰-substituted or unsubstituted alkyl, R³⁰-substituted or unsubstituted heteroalkyl, R³⁰-substituted or unsubstituted cycloalkyl, R³⁰-substituted or unsubstituted heterocycloalkyl, R³⁰-substituted or unsubstituted aryl, or R³⁰-substituted or unsubstituted heteroaryl. R²⁹ may be R³⁰-substituted or unsubstituted C₁-C₂₀ (e.g., C₁-C₆) alkyl, R³⁰-substituted or unsubstituted 2 to 20 membered (e.g., 2 to 6 membered) heteroalkyl, R³⁰-substituted or unsubstituted C₃-C₈ (e.g., C₅-C₇) cykloalkyl, R³⁰-substituted or unsubstituted 3 to 8 membered (e.g., 3 to 6 membered) heterocycloalkyl, R³⁰-substituted or unsubstituted C₅-C₁₀ (e.g., C₅-C₆) aryl, or R³⁰-substituted or unsubstituted 5 to 10 membered (e.g., 5 to 6 membered) heteroaryl.

R³⁰ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R³⁰ is ═O, R²⁹ is not aryl or heteroaryl. In some embodiments, R³⁰ is R³¹-substituted or unsubstituted alkyl, R³¹-substituted or unsubstituted heteroalkyl, R³¹-substituted or unsubstituted cycloalkyl, R³¹-substituted or unsubstituted heterocycloalkyl, R³¹-substituted or unsubstituted aryl, or R³¹-substituted or unsubstituted heteroaryl. R³⁰ may be R³¹-substituted or unsubstituted C₁-C₂₀ (e.g., C₁-C₆) alkyl, R³¹-substituted or unsubstituted 2 to 20 membered (e.g., 2 to 6 membered) heteroalkyl, R³¹-substituted or unsubstituted C₃-C₈ (e.g., C₅-C₇) cykloalkyl, R³¹-substituted or unsubstituted 3 to 8 membered (e.g., 3 to 6 membered) heterocycloalkyl, R³¹-substituted or unsubstituted C₅-C₁₀ (e.g., C₅-C₆) aryl, or R³¹-substituted or unsubstituted 5 to 10 membered (e.g., 5 to 6 membered) heteroaryl.

R³¹ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R³¹ is ═O, R³⁰ is not aryl or heteroaryl. In some embodiments, R³¹ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In other embodiments, R⁵ is —NHR⁷. R⁷ may be hydrogen, substituted or unsubstituted C₁-C₂₀ alkyl, substituted or unsubstituted 2 to 20 membered heteroalkyl, C₃-C₈ cykloalkyl, substituted or unsubstituted 3 to 8 membered heterocycloalkyl, substituted or unsubstituted C₅-C₁₀ aryl, or substituted or unsubstituted 5 to 10 membered heteroaryl. In some embodiments, R⁷ is substituted or unsubstituted C₁-C₂₀ alkyl. In some embodiments, R⁷ is substituted or unsubstituted C₁-C₂₀ (e.g., C₁-C₁₀) alkyl. In other embodiments, R⁷ is substituted or unsubstituted C₁-C₁₀ (e.g., C₁-C₆) alkyl. In other embodiments, R⁷ is substituted or unsubstituted C₁-C₄ (e.g., C₁-C₂) alkyl. In some embodiments, R⁷ is unsubstituted C₁-C₄ (e.g., C₁-C₂) alkyl. In some embodiments, R⁷ is methyl or ethyl. In other embodiments, R⁷ is methyl.

In some embodiments, R⁸ is hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R³²-substituted or unsubstituted alkyl, R³²-substituted or unsubstituted heteroalkyl, R³²-substituted or unsubstituted cycloalkyl, R³²-substituted or unsubstituted heterocycloalkyl, R³²-substituted or unsubstituted aryl, or R³²-substituted or unsubstituted heteroaryl. R⁸ may be hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R³²-substituted or unsubstituted C₁-C₂₀ (e.g., C₁-C₆) alkyl, R³²-substituted or unsubstituted 2 to 20 membered (e.g., 2 to 6 membered) heteroalkyl, R³²-substituted or unsubstituted C₃-C₈ (e.g., C₅-C₇) cykloalkyl, R³²-substituted or unsubstituted 3 to 8 membered (e.g., 3 to 6 membered) heterocycloalkyl, R³²-substituted or unsubstituted C₅-C₁₀ (e.g., C₅-C₆) aryl, or R³²-substituted or unsubstituted 5 to 10 membered (e.g., 5 to 6 membered) heteroaryl.

R³² is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R³² is ═O, R⁸ is not aryl or heteroaryl. In some embodiments, R³² is R³³-substituted or unsubstituted alkyl, R³³-substituted or unsubstituted heteroalkyl, R³³-substituted or unsubstituted cycloalkyl, R³³-substituted or unsubstituted heterocycloalkyl, R³³-substituted or unsubstituted aryl, or R³³-substituted or unsubstituted heteroaryl. R³² may be R³³-substituted or unsubstituted C₁-C₂₀ (e.g., C₁-C₆) alkyl, R³³-substituted or unsubstituted 2 to 20 membered (e.g., 2 to 6 membered) heteroalkyl, R³³-substituted or unsubstituted C₃-C₈ (e.g., C₅-C₇) cykloalkyl, R³³-substituted or unsubstituted 3 to 8 membered (e.g., 3 to 6 membered) heterocycloalkyl, R³³-substituted or unsubstituted C₅-C₁₀ (e.g., C₅-C₆) aryl, or R³³-substituted or unsubstituted 5 to 10 membered (e.g., 5 to 6 membered) heteroaryl.

R³³ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R³³ is ═O, R³² is not aryl or heteroaryl. In some embodiments, R³³ is R³⁴-substituted or unsubstituted alkyl, R³⁴-substituted or unsubstituted heteroalkyl, R³⁴-substituted or unsubstituted cycloalkyl, R³⁴-substituted or unsubstituted heterocycloalkyl, R³⁴-substituted or unsubstituted aryl, or R³⁴-substituted or unsubstituted heteroaryl. R³³ may be R³⁴-substituted or unsubstituted C₁-C₂₀ (e.g., C₁-C₆) alkyl, R³⁴-substituted or unsubstituted 2 to 20 membered (e.g., 2 to 6 membered) heteroalkyl, R³⁴-substituted or unsubstituted C₃-C₈ (e.g., C₅-C₇) cykloalkyl, R³⁴-substituted or unsubstituted 3 to 8 membered (e.g., 3 to 6 membered) heterocycloalkyl, R³⁴-substituted or unsubstituted C₅-C₁₀ (e.g., C₅-C₆) aryl, or R³⁴-substituted or unsubstituted 5 to 10 membered (e.g., 5 to 6 membered) heteroaryl.

R³⁴ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R³⁴ is ═O, R³³ is not aryl or heteroaryl. In some embodiments, R³⁴ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In some embodiments, R⁹ is hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R³⁵-substituted or unsubstituted alkyl, R³⁵-substituted or unsubstituted heteroalkyl, R³⁵-substituted or unsubstituted cycloalkyl, R³⁵-substituted or unsubstituted heterocycloalkyl, R³⁵-substituted or unsubstituted aryl, or R³⁵-substituted or unsubstituted heteroaryl. R⁹ may be hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R³⁵-substituted or unsubstituted C₁-C₂₀ (e.g., C₁-C₆) alkyl, R³⁵-substituted or unsubstituted 2 to 20 membered (e.g., 2 to 6 membered) heteroalkyl, R³⁵-substituted or unsubstituted C₃-C₈ (e.g., C₅-C₇) cykloalkyl, R³⁵-substituted or unsubstituted 3 to 8 membered (e.g., 3 to 6 membered) heterocycloalkyl, R³⁵-substituted or unsubstituted C₅-C₁₀ (e.g., C₅-C₆) aryl, or R³⁵-substituted or unsubstituted 5 to 10 membered (e.g., 5 to 6 membered) heteroaryl.

R³⁵ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R³⁵ is ═O, R⁹ is not aryl or heteroaryl. In some embodiments, R³⁵ is R³⁶-substituted or unsubstituted alkyl, R³⁶-substituted or unsubstituted heteroalkyl, R³⁶-substituted or unsubstituted cycloalkyl, R³⁶-substituted or unsubstituted heterocycloalkyl, R³⁶-substituted or unsubstituted aryl, or R³⁶-substituted or unsubstituted heteroaryl. R³⁵ may be R³⁶-substituted or unsubstituted C₁-C₂₀ (e.g., C₁-C₆) alkyl, R³⁶-substituted or unsubstituted 2 to 20 membered (e.g., 2 to 6 membered) heteroalkyl, R³⁶-substituted or unsubstituted C₃-C₈ (e.g., C₅-C₇) cykloalkyl, R³⁶-substituted or unsubstituted 3 to 8 membered (e.g., 3 to 6 membered) heterocycloalkyl, R³⁶-substituted or unsubstituted C₅-C₁₀ (e.g., C₅-C₆) aryl, or R³⁶-substituted or unsubstituted 5 to 10 membered (e.g., 5 to 6 membered) heteroaryl.

R³⁶ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R³⁶ is ═O, R³⁵ is not aryl or heteroaryl. In some embodiments, R³⁶ is R³⁷-substituted or unsubstituted alkyl, R³⁷-substituted or unsubstituted heteroalkyl, R³⁷-substituted or unsubstituted cycloalkyl, R³⁷-substituted or unsubstituted heterocycloalkyl, R³⁷-substituted or unsubstituted aryl, or R³⁷-substituted or unsubstituted heteroaryl. R³⁶ may be R³⁷-substituted or unsubstituted C₁-C₂₀ (e.g., C₁-C₆) alkyl, R³⁷-substituted or unsubstituted 2 to 20 membered (e.g., 2 to 6 membered) heteroalkyl, R³⁷-substituted or unsubstituted C₃-C₈ (e.g., C₅-C₇) cykloalkyl, R³⁷-substituted or unsubstituted 3 to 8 membered (e.g., 3 to 6 membered) heterocycloalkyl, R³⁷-substituted or unsubstituted C₅-C₁₀ (e.g., C₅-C₆) aryl, or R³⁷-substituted or unsubstituted 5 to 10 membered (e.g., 5 to 6 membered) heteroaryl.

R³⁷ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R³⁷ is ═O, R³⁶ is not aryl or heteroaryl. In some embodiments, R³⁷ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In some embodiments, R¹⁰ is hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R³⁸-substituted or unsubstituted alkyl, R³⁸-substituted or unsubstituted heteroalkyl, R³⁸-substituted or unsubstituted cycloalkyl, R³⁸-substituted or unsubstituted heterocycloalkyl, R³⁸-substituted or unsubstituted aryl, or R³⁸-substituted or unsubstituted heteroaryl. R¹⁰ may be hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, R³⁸-substituted or unsubstituted C₁-C₂₀ (e.g., C₁-C₆) alkyl, R³⁸-substituted or unsubstituted 2 to 20 membered (e.g., 2 to 6 membered) heteroalkyl, R³⁸-substituted or unsubstituted C₃-C₈ (e.g., C₅-C₇) cykloalkyl, R³⁸-substituted or unsubstituted 3 to 8 membered (e.g., 3 to 6 membered) heterocycloalkyl, R³⁸-substituted or unsubstituted C₅-C₁₀ (e.g., C₅-C₆) aryl, or R³⁸-substituted or unsubstituted 5 to 10 membered (e.g., 5 to 6 membered) heteroaryl.

R³⁸ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R³⁸ is ═O, R¹⁰ is not aryl or heteroaryl. In some embodiments, R³⁸ is R³⁹-substituted or unsubstituted alkyl, R³⁹-substituted or unsubstituted heteroalkyl, R³⁹-substituted or unsubstituted cycloalkyl, R³⁹-substituted or unsubstituted heterocycloalkyl, R³⁹-substituted or unsubstituted aryl, or R³⁹-substituted or unsubstituted heteroaryl. R³⁸ may be R³⁹-substituted or unsubstituted C₁-C₂₀ (e.g., C₁-C₆) alkyl, R³⁹-substituted or unsubstituted 2 to 20 membered (e.g., 2 to 6 membered) heteroalkyl, R³⁹-substituted or unsubstituted C₃-C₈ (e.g., C₅-C₇) cykloalkyl, R³⁹-substituted or unsubstituted 3 to 8 membered (e.g., 3 to 6 membered) heterocycloalkyl, R³⁹-substituted or unsubstituted C₅-C₁₀ (e.g., C₅-C₆) aryl, or R³⁹-substituted or unsubstituted 5 to 10 membered (e.g., 5 to 6 membered) heteroaryl.

R³⁹ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R³⁹ is ═O, R³⁸ is not aryl or heteroaryl. In some embodiments, R³⁹ is R⁴⁰-substituted or unsubstituted alkyl, R⁴⁰-substituted or unsubstituted heteroalkyl, R⁴⁰-substituted or unsubstituted cycloalkyl, R⁴⁰-substituted or unsubstituted heterocycloalkyl, R⁴⁰-substituted or unsubstituted aryl, or R⁴⁰-substituted or unsubstituted heteroaryl. R³⁹ may be R⁴⁰-substituted or unsubstituted C₁-C₂₀ (e.g., C₁-C₆) alkyl, R⁴⁰-substituted or unsubstituted 2 to 20 membered (e.g., 2 to 6 membered) heteroalkyl, R⁴⁰-substituted or unsubstituted C₃-C₈ (e.g., C₅-C₇) cykloalkyl, R⁴⁰-substituted or unsubstituted 3 to 8 membered (e.g., 3 to 6 membered) heterocycloalkyl, R⁴⁰-substituted or unsubstituted C₅-C₁₀ (e.g., C₅-C₆) aryl, or R⁴⁰-substituted or unsubstituted 5 to 10 membered (e.g., 5 to 6 membered) heteroaryl.

R⁴⁰ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R⁴⁰ is ═O, R³⁹ is not aryl or heteroaryl. In some embodiments, R⁴⁰ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

L¹, L², L³ and L⁴ may be the same or different and may each independently be a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted, or unsubstituted heteroalkylene, substituted or unsubstituted cycloalkylene, substituted or unsubstituted heterocycloalkylene, substituted or unsubstituted arylene, or substituted or unsubstituted heteroarylene. In some embodiments, L¹, L², L³ and L⁴ are independently a bond, —C(O)O—, —OC(O)—, —C(O)—, —C(O)NH—, —NH—, —NHC(O)—, —O—, —S—, substituted or unsubstituted alkylene, substituted, or unsubstituted heteroalkylene, substituted or unsubstituted cycloalkylene, substituted or unsubstituted heterocykloalkylene, substituted or unsubstituted arylene, or substituted or unsubstituted heteroarylene.

In some embodiments, L¹ is a bond, —S(O)—, —S(O)₂NH—, —NHS(O)₂—, —C(O)O—, —OC(O)—, —C(O)—, —C(O)NH—, —NH—, —NHC(O)—, —I—, —S—, R⁴¹-substituted or unsubstituted alkylene, R⁴¹-substituted or unsubstituted heteroalkylene, R⁴¹-substituted or unsubstituted cycloalkylene, R⁴¹-substituted or unsubstituted heterocycloalkylene, R⁴¹-substituted or unsubstituted arylene, or R⁴¹-substituted or unsubstituted heteroarylene. L¹ may be a bond, —S(O)—, —S(O)₂NH—, —NHS(O)₂—, —C(O)O—, —OC(O)—, —C(O)—, —C(O)NH—, —NH—, —NHC(O)—, —O—, —S—, R⁴¹-substituted or unsubstituted C₁-C₂₀ (e.g., C₁-C₆) alkylene, R⁴¹-substituted or unsubstituted 2 to 20 membered (e.g., 2 to 6 membered) heteroalkylene, R⁴¹-substituted or unsubstituted C₃-C₈ (e.g., C₅-C₇) cykloalkylene, R⁴¹-substituted or unsubstituted 3 to 8 membered (e.g., 3 to 6 membered) heterocycloalkylene, R⁴¹-substituted or unsubstituted C₅-C₁₀ (e.g., C₅-C₆) arylene, or R⁴¹-substituted or unsubstituted 5 to 10 membered (e.g., 5 to 6 membered) heteroarylene.

R⁴¹ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R⁴¹ is ═O, L¹ is not arylene or heteroarylene. In some embodiments, R⁴¹ is R⁴²-substituted or unsubstituted alkyl, R⁴²-substituted or unsubstituted heteroalkyl, R⁴²-substituted or unsubstituted cycloalkyl, R⁴²-substituted or unsubstituted heterocycloalkyl, R⁴²-substituted or unsubstituted aryl, or R⁴²-substituted or unsubstituted heteroaryl. R⁴¹ may be R⁴²-substituted or unsubstituted C₁-C₂₀ (e.g., C₁-C₆) alkyl, R⁴²-substituted or unsubstituted 2 to 20 membered (e.g., 2 to 6 membered) heteroalkyl, R⁴²-substituted or unsubstituted C₃-C₈ (e.g., C₅-C₇) cykloalkyl, R⁴²-substituted or unsubstituted 3 to 8 membered (e.g., 3 to 6 membered) heterocycloalkyl, R⁴²-substituted or unsubstituted C₅-C₁₀ (e.g., C₅-C₆) aryl, or R⁴²-substituted or unsubstituted 5 to 10 membered (e.g., 5 to 6 membered) heteroaryl.

R⁴² is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R⁴² is ═O, R⁴¹ is not aryl or heteroaryl. In some embodiments, R⁴² is R⁴³-substituted or unsubstituted alkyl, R⁴³-substituted or unsubstituted heteroalkyl, R⁴³-substituted or unsubstituted cycloalkyl, R⁴³-substituted or unsubstituted heterocycloalkyl, R⁴³-substituted or unsubstituted aryl, or R⁴³-substituted or unsubstituted heteroaryl. R⁴² may be R⁴³-substituted or unsubstituted C₁-C₂₀ (e.g., C₁-C₆) alkyl, R⁴³-substituted or unsubstituted 2 to 20 membered (e.g., 2 to 6 membered) heteroalkyl, R⁴³-substituted or unsubstituted C₃-C₈ (e.g., C₅-C₇) cykloalkyl, R⁴³-substituted or unsubstituted 3 to 8 membered (e.g., 3 to 6 membered) heterocycloalkyl, R⁴³-substituted or unsubstituted C₅-C₁₀ (e.g., C₅-C₆) aryl, or R⁴³-substituted or unsubstituted 5 to 10 membered (e.g., 5 to 6 membered) heteroaryl.

R⁴³ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R⁴³ is ═O, R⁴² is not aryl or heteroaryl. In some embodiments, R⁴³ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In some embodiments, L² is a bond, —S(O)—, —S(O)₂NH—, —NHS(O)₂—, —C(O)O—, —OC(O)—, —C(O)—, —C(O)NH—, —NH—, —NHC(O)—, —O—, —S—, R⁴⁴-substituted or unsubstituted alkylene, R⁴⁴-substituted or unsubstituted heteroalkylene, R⁴⁴-substituted or unsubstituted cycloalkylene, R⁴⁴-substituted or unsubstituted heterocycloalkylene, R⁴⁴-substituted or unsubstituted arylene, or R⁴⁴-substituted or unsubstituted heteroarylene. L² may be a bond, —S(O)—, —S(O)₂NH—, —NHS(O)₂—, —C(O)O—, —OC(O)—, —C(O)—, —C(O)NH—, —NH—, —NHC(O)—, —O—, —S—, R⁴⁴-substituted or unsubstituted C₁-C₂₀ (e.g., C₁-C₆) alkylene, R⁴⁴-substituted or unsubstituted 2 to 20 membered (e.g., 2 to 6 membered) heteroalkylene, R⁴⁴-substituted or unsubstituted C₃-C₈ (e.g., C₅-C₇) cykloalkylene, R⁴⁴-substituted or unsubstituted 3 to 8 membered (e.g., 3 to 6 membered) heterocycloalkylene, R⁴⁴-substituted or unsubstituted C₅-C₁₀ (e.g., C₅-C₆) arylene, or R⁴⁴-substituted or unsubstituted 5 to 10 membered (e.g., 5 to 6 membered) heteroarylene.

R⁴⁴ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R⁴⁴ is ═O, L² is not arylene or heteroarylene. In some embodiments, R⁴⁴ is R⁴⁵-substituted or unsubstituted alkyl, R⁴⁵-substituted or unsubstituted heteroalkyl, R⁴⁵-substituted or unsubstituted cycloalkyl, R⁴⁵-substituted or unsubstituted heterocycloalkyl, R⁴⁵-substituted or unsubstituted aryl, or R⁴⁵-substituted or unsubstituted heteroaryl. R⁴⁴ may be R⁴⁵-substituted or unsubstituted C₁-C₂₀ (e.g., C₁-C₆) alkyl, R⁴⁵-substituted or unsubstituted 2 to 20 membered (e.g., 2 to 6 membered) heteroalkyl, R⁴⁵-substituted or unsubstituted C₃-C₈ (e.g., C₅-C₇) cykloalkyl, R⁴⁵-substituted or unsubstituted 3 to 8 membered (e.g., 3 to 6 membered) heterocycloalkyl, R⁴⁵-substituted or unsubstituted C₅-C₁₀ (e.g., C₅-C₆) aryl, or R⁴⁵-substituted or unsubstituted 5 to 10 membered (e.g., 5 to 6 membered) heteroaryl.

R⁴⁵ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R⁴⁵ is ═O, R⁴⁴ is not aryl or heteroaryl. In some embodiments, R⁴⁵ is R⁴⁶-substituted or unsubstituted alkyl, R⁴⁶-substituted or unsubstituted heteroalkyl, R⁴⁶-substituted or unsubstituted cycloalkyl, R⁴⁶-substituted or unsubstituted heterocycloalkyl, R⁴⁶-substituted or unsubstituted aryl, or R⁴⁶-substituted or unsubstituted heteroaryl. R⁴⁵ may be R⁴⁶-substituted or unsubstituted C₁-C₂₀ (e.g., C₁-C₆) alkyl, R⁴⁶-substituted or unsubstituted 2 to 20 membered (e.g., 2 to 6 membered) heteroalkyl, R⁴⁶-substituted or unsubstituted C₃-C₈ (e.g., C₅-C₇) cykloalkyl, R⁴⁶-substituted or unsubstituted 3 to 8 membered (e.g., 3 to 6 membered) heterocycloalkyl, R⁴⁶-substituted or unsubstituted C₅-C₁₀ (e.g., C₅-C₆) aryl, or R⁴⁶-substituted or unsubstituted 5 to 10 membered (e.g., 5 to 6 membered) heteroaryl.

R⁴⁶ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R⁴⁶ is ═O, R⁴⁵ is not aryl or heteroaryl. In some embodiments, R⁴⁶ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In some embodiments, L³ is a bond, —S(O)—, —S(O)₂NH—, —NHS(O)₂—, —C(O)O—, —OC(O)—, —C(O)—, —C(O)NH—, —NH—, —NHC(O)—, —O—, —S—, R⁴⁷-substituted or unsubstituted alkylene, R⁴⁷-substituted or unsubstituted heteroalkylene, R⁴⁷-substituted or unsubstituted cycloalkylene, R⁴⁷-substituted or unsubstituted heterocycloalkylene, R⁴⁷-substituted or unsubstituted arylene, or R⁴⁷-substituted or unsubstituted heteroarylene. L³ may be a bond, —S(O)—, —S(O)₂NH—, —NHS(O)₂—, —C(O)O—, —OC(O)—, —C(O)—, —C(O)NH—, —NH—, —NHC(O)—, —O—, —S—, R⁴⁷-substituted or unsubstituted C₁-C₂₀ (e.g., C₁-C₆) alkylene, R⁴⁷-substituted or unsubstituted 2 to 20 membered (e.g., 2 to 6 membered) heteroalkylene, R⁴⁷-substituted or unsubstituted C₃-C₈ (e.g., C₅-C₇) cykloalkylene, R⁴⁷-substituted or unsubstituted 3 to 8 membered (e.g., 3 to 6 membered) heterocycloalkylene, R⁴⁷-substituted or unsubstituted C₅-C₁₀ (e.g., C₅-C₆) arylene, or R⁴⁷-substituted or unsubstituted 5 to 10 membered (e.g., 5 to 6 membered) heteroarylene.

R⁴⁷ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R⁴⁷ is ═O, L³ is not arylene or heteroarylene. In some embodiments, R⁴⁷ is R⁴⁸-substituted or unsubstituted alkyl, R⁴⁸-substituted or unsubstituted heteroalkyl, R⁴⁸-substituted or unsubstituted cycloalkyl, R⁴⁸-substituted or unsubstituted heterocycloalkyl, R⁴⁸-substituted or unsubstituted aryl, or R⁴⁸-substituted or unsubstituted heteroaryl. R⁴⁷ may be R⁴⁸-substituted or unsubstituted C₁-C₂₀ (e.g., C₁-C₆) alkyl, R⁴⁸-substituted or unsubstituted 2 to 20 membered (e.g., 2 to 6 membered) heteroalkyl, R⁴⁸-substituted or unsubstituted C₃-C₈ (e.g., C₅-C₇) cykloalkyl, R⁴⁸-substituted or unsubstituted 3 to 8 membered (e.g., 3 to 6 membered) heterocycloalkyl, R⁴⁸-substituted or unsubstituted C₅-C₁₀ (e.g., C₅-C₆) aryl, or R⁴⁸-substituted or unsubstituted 5 to 10 membered (e.g., 5 to 6 membered) heteroaryl.

R⁴⁸ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R⁴⁸ is ═O, R⁴⁷ is not aryl or heteroaryl. In some embodiments, R⁴⁸ is R⁴⁹-substituted or unsubstituted alkyl, R⁴⁹-substituted or unsubstituted heteroalkyl, R⁴⁹-substituted or unsubstituted cycloalkyl, R⁴⁹-substituted or unsubstituted heterocycloalkyl, R⁴⁹-substituted or unsubstituted aryl, or R⁴⁹-substituted or unsubstituted heteroaryl. R⁴⁸ may be R⁴⁹-substituted or unsubstituted C₁-C₂₀ (e.g., C₁-C₆) alkyl, R⁴⁹-substituted or unsubstituted 2 to 20 membered (e.g., 2 to 6 membered) heteroalkyl, R⁴⁹-substituted or unsubstituted C₃-C₈ (e.g., C₅-C₇) cykloalkyl, R⁴⁹-substituted or unsubstituted 3 to 8 membered (e.g., 3 to 6 membered) heterocycloalkyl, R⁴⁹-substituted or unsubstituted C₅-C₁₀ (e.g., C₅-C₆) aryl, or R⁴⁹-substituted or unsubstituted 5 to 10 membered (e.g., 5 to 6 membered) heteroaryl.

R⁴⁹ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R⁴⁹ is ═O, R⁴⁸ is not aryl or heteroaryl. In some embodiments, R⁴⁹ is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In some embodiments, L⁴ is a bond, —S(O)—, —S(O)₂NH—, —NHS(O)₂—, —C(O)O—, —OC(O)—, —C(O)—, —C(O)NH—, —NH—, —NHC(O)—, —O—, —S—, R⁵⁰-substituted or unsubstituted alkylene, R⁵⁰-substituted or unsubstituted heteroalkylene, R⁵⁰-substituted or unsubstituted cycloalkylene, R⁵⁰-substituted or unsubstituted heterocycloalkylene, R⁵⁰-substituted or unsubstituted arylene, or R⁵⁰-substituted or unsubstituted heteroarylene. L⁴ may be a bond, —S(O)—, —S(O)₂NH—, —NHS(O)₂—, —C(O)O—, —OC(O)—, —C(O)—, —C(O)NH—, —NH—, —NHC(O)—, —O—, —S—, R⁵⁰-substituted or unsubstituted C₁-C₂₀ (e.g., C₁-C₆) alkylene, R⁵⁰-substituted or unsubstituted 2 to 20 membered (e.g., 2 to 6 membered) heteroalkylene, R⁵⁰-substituted or unsubstituted C₃-C₈ (e.g., C₅-C₇) cykloalkylene, R⁵⁰-substituted or unsubstituted 3 to 8 membered (e.g., 3 to 6 membered) heterocycloalkylene, R⁵⁰-substituted or unsubstituted C₅-C₁₀ (e.g., C₅-C₆) arylene, or R⁵⁰-substituted or unsubstituted 5 to 10 membered (e.g., 5 to 6 membered) heteroarylene.

R⁵⁰ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R⁵⁰ is ═O, L⁴ is not arylene or heteroarylene. In some embodiments, R⁵⁰ is R⁵¹-substituted or unsubstituted alkyl, R⁵¹-substituted or unsubstituted heteroalkyl, R⁵¹-substituted or unsubstituted cycloalkyl, R⁵¹-substituted or unsubstituted heterocycloalkyl, R⁵¹-substituted or unsubstituted aryl, or R⁵¹-substituted or unsubstituted heteroaryl. R⁵⁰ may be R⁵¹-substituted or unsubstituted C₁-C₂₀ (e.g., C₁-C₆) alkyl, R⁵¹-substituted or unsubstituted 2 to 20 membered (e.g., 2 to 6 membered) heteroalkyl, R⁵¹-substituted or unsubstituted C₃-C₈ (e.g., C₅-C₇) cykloalkyl, R⁵¹-substituted or unsubstituted 3 to 8 membered (e.g., 3 to 6 membered) heterocycloalkyl, R⁵¹-substituted or unsubstituted C₅-C₁₀ (e.g., C₅-C₆) aryl, or R⁵¹-substituted or unsubstituted 5 to 10 membered (e.g., 5 to 6 membered) heteroaryl.

R⁵¹ is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, or —NHC═(O)NHNH₂. In some embodiments, where R⁵¹ is ═O, R⁵⁰ is not aryl or heteroaryl. In some embodiments, R⁵¹ is R⁵²-substituted or unsubstituted alkyl, R⁵²-substituted or unsubstituted heteroalkyl, R⁵²-substituted or unsubstituted cycloalkyl, R⁵²-substituted or unsubstituted heterocycloalkyl, R⁵²-substituted or unsubstituted aryl, or R⁵²-substituted or unsubstituted heteroaryl. R⁵¹ may be R⁵²-substituted or unsubstituted C₁-C₂₀ (e.g., C₁-C₆) alkyl, R⁵²-substituted or unsubstituted 2 to 20 membered (e.g., 2 to 6 membered) heteroalkyl, R⁵²-substituted or unsubstituted C₃-C₈ (e.g., C₅-C₇) cykloalkyl, R⁵²-substituted or unsubstituted 3 to 8 membered (e.g., 3 to 6 membered) heterocycloalkyl, R⁵²-substituted or unsubstituted C₅-C₁₀ (e.g., C₅-C₆) aryl, or R⁵²-substituted or unsubstituted 5 to 10 membered (e.g., 5 to 6 membered) heteroaryl.

R⁵² is halogen, ═O (oxo), —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, unsubstituted alkyl, unsubstituted heteroalkyl, unsubstituted cycloalkyl, unsubstituted heterocycloalkyl, unsubstituted aryl, or unsubstituted heteroaryl. In some embodiments, where R⁵² is ═O, R⁵¹ is not aryl or heteroaryl. In some embodiments, R⁵² is unsubstituted C₁-C₂₀ (e.g., C₁-C₆) alkyl, unsubstituted 2 to 20 membered (e.g., 2 to 6 membered) heteroalkyl, unsubstituted C₃-C₈ (e.g., C₅-C₇) cykloalkyl, unsubstituted 3 to 8 membered (e.g., 3 to 6 membered) heterocycloalkyl, unsubstituted C₅-C₁₀ (e.g., C₅-C₆) aryl, or unsubstituted 5 to 10 membered (e.g., 5 to 6 membered) heteroaryl.

In the embodiments provided herein L¹, L², L³ and L⁴ may be independently a bond, —C(O)NH—, substituted or unsubstituted C₁-C₁₀ alkylene, or substituted or unsubstituted 2 to 10 membered heteroalkylene (e.g., including R-substituted or unsubstituted embodiments as set forth above). In other embodiments, L¹, L³ and L⁴ are independently a bond or substituted or unsubstituted C₁-C₁₀ (e.g., C₁-C₈) alkylene. In some embodiments, L¹, L³ and L⁴ are independently a bond or substituted or unsubstituted C₁-C₆ (e.g., C₁-C₄) alkylene. In some embodiments, L¹, L³ and L⁴ are independently a bond or substituted or unsubstituted C₁-C₄ (e.g., C₁-C₃) alkylene. In some embodiments, L¹, L³ and L⁴ are independently a bond or unsubstituted C₁-C₄ (e.g., C₁-C₃) alkylene. In some embodiments, L¹, L³ and L⁴ are independently a bond, ethylene or methylene. In some embodiments, L¹, L³ and L⁴ are a bond. In other embodiments, L¹, L³ and L⁴ are methylene. In some embodiments, L³ is —C(O)NH—.

In some embodiments, the compound is having the structure of Formula (II). L¹ is a bond, R¹ is halophenyl, X^{3′} is —N(-L²-R²), L²-R² is

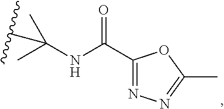 [SMILES: *C(C)(C)NC(=O)c1nnc(C)o1]

L³ is a bond, R³ is hydrogen, L⁴ is a bond, and R⁴ is methyl.

In other embodiments, the compound is having the structure of Formula (IV). L¹ is a bond, R¹ is halophenyl, L²-R² is

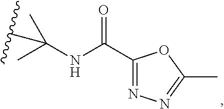 [SMILES: *C(C)(C)NC(=O)c1nnc(C)o1]

L³ is —C(O)NH—, R³ is methyl, L⁴ is a bond, and R⁴ is hydrogen.

Further to any of Formulae (I) to (XV), in some embodiments a substituent is a size-limited substituent. For example without limitation, in some embodiments each substituted or unsubstituted alkyl may be a substituted or unsubstituted C₁-C₂₀, C₁-C₁₀, C₁-C₆, or even C₁ alkyl. In some embodiments each substituted or unsubstituted heteroalkyl may be a substituted or unsubstituted 2-20 membered, 2-10 membered, or 2-6 membered heteroalkyl. In some embodiments, each substituted or unsubstituted cycloalkyl is a substituted or unsubstituted C₃-C₈, C₄-C₈, C₅-C₇ cycloalkyl. In some embodiments, each substituted or unsubstituted heterocycloalkyl is a substituted or unsubstituted 3-8 membered, 4-8 membered, or 3-6 membered heterocycloalkyl. In some embodiments, each substituted or unsubstituted heteroaryl is a substituted or unsubstituted 4-14 membered, 4-10 membered, 5-8 membered, 4-6 membered, 5-6 membered, or 6-membered heteroaryl. In some embodiments, each substituted or unsubstituted aryl is a substituted or unsubstituted C₄-C₁₄, C₄-C₁₀, C₆-C₁₀, C₅-C₈, C₅-C₆, or C₆ aryl (phenyl). In other embodiments each substituted or unsubstituted alkylene may be a substituted or unsubstituted C₁-C₂₀, C₁-C₁₀, C₁-C₆, or even C₁ alkylene. In some embodiments each substituted or unsubstituted heteroalkylene may be a substituted or unsubstituted 2-20 membered, 2-10 membered, or 2-6 membered heteroalkylene. In some embodiments, each substituted or unsubstituted cycloalkylene is a substituted or unsubstituted C₃-C₈, C₄-C₈, C₅-C₇ cycloalkylene. In some embodiments, each substituted or unsubstituted heterocycloalkylene is a substituted or unsubstituted 3-8 membered, 4-8 membered, or 3-6 membered heterocycloalkylene. In some embodiments, each substituted or unsubstituted heteroarylene is a substituted or unsubstituted 4-14 membered, 4-10 membered, 5-8 membered, 4-6 membered, 5-6 membered, or 6-membered heteroarylene. In some embodiments, each substituted or unsubstituted arylene is a substituted or unsubstituted C₄-C₁₄, C₄-C₁₀, C₆-C₁₀, C₅-C₈, C₅-C₆, or C₆ arylene (phenylene).

In another aspect, a pharmaceutical composition is provided. The pharmaceutical composition includes a pharmaceutically acceptable excipient and a compound provided herein including embodiments thereof.

Agents of the invention are often administered as pharmaceutical compositions comprising an active therapeutic agent, i.e., and a variety of other pharmaceutically acceptable components. See Remington's Pharmaceutical Science (15th ed., Mack Publishing Company, Easton, Pa., 1980). The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

The compositions can be administered for therapeutic or prophylactic treatments. In therapeutic applications, compositions are administered to a patient suffering from a disease (e.g., HIV infection, AIDS) in a “therapeutically effective dose.” Amounts effective for this use will depend upon the severity of the disease and the general state of the patient's health. Single or multiple administrations of the compositions may be administered depending on the dosage and frequency as required and tolerated by the patient. A “patient” or “subject” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

The compositions provided herein, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administrations contemplates coadministration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating HIV infection using HIV integrase inhibitors for guidance.

### III. Methods of Treatment

Provided herein are methods of treating infectious diseases. In one aspect, a method of treating an infectious disease in a subject in need thereof is provided. The method includes administering to the subject a therapeutically effective amount of a compound provided herein including embodiments thereof. In some embodiments, the infectious disease is caused by a virus. In some further embodiments, the virus is HIV. In other embodiments, the subject suffers from AIDS. Thus, in some embodiments, provided herein is a method of treating HIV infection in a subject infected with HIV, wherein the method includes administering to the subject a therapeutically effective amount of a compound provided herein including embodiments thereof. In other embodiments, provided herein is a method of treating AIDS in a subject in need thereof, wherein the method includes administering to the subject a therapeutically effective amount of a compound provided herein including embodiments thereof.

In one aspect, a method of inhibiting HIV integrase in a patient is provided. The method includes administering to the patient a therapeutically effective amount of a compound provided herein including embodiments thereof thereby HIV integrase in the patient.

In another aspect, a method of inhibiting HIV integrase is provided. The method includes contacting HIV integrase with an effective amount of a compound provided herein including embodiments thereof thereby inhibiting the HIV integrase.

In another aspect, a method of inhibiting HIV integrase in vitro is provided. The method includes contacting HIV integrase in vitro with an effective amount of a compound provided herein including embodiments thereof thereby inhibiting the HIV integrase.

### IV. Examples

In an attempt to better understand the key metal-ligand interactions involved in HIV-1 IN inhibition, a series of raltegravir-chelator derivatives (RCDs) have been synthesized and evaluated. These compounds were designed to systematically examine the inhibitory effect of each MBG by keeping the remainder of the inhibitor structure unaltered. This was achieved by appending various MBGs to the p-fluorobenzyl backbone via a carboxyamide linkage, the latter of which provides the first of the three donor atoms. These INSTIs were screened against HIV-1 IN to determine which metal-binding groups (MBGs) produced inhibitors with comparable or better activity than an abbreviated raltegravir derivative (RCD-1). Several RCDs had comparable strand-transfer inhibitory activity to RCD-1 and two derivatives, containing a hydroxypyrone MBG, were more effective at inhibiting strand transfer. Computational docking studies of RCDs in the active site of PFV IN have been performed to elucidate key features that contribute to effective metal chelation to the HIV-1 IN active site. The findings presented here are the first to systematically investigate and rigorously analyze the importance of different MBGs in HIV-1 IN.

Design and Synthesis of Inhibitors

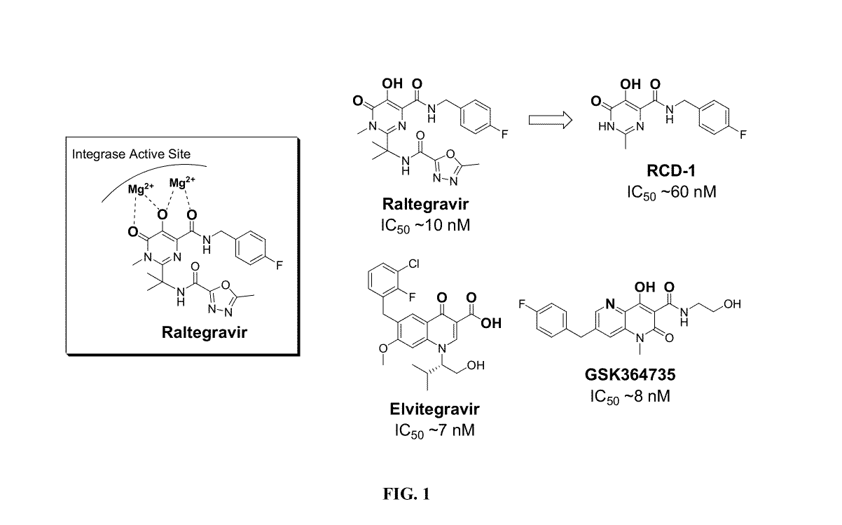

In order to isolate and examine the effect of the MBG in HIV-1 IN inhibitors, a series of RCDs were designed and synthesized. These INSTIs are identical to a core portion of raltegravir and vary only in the nature of the MBG. The RCDs that were prepared are shown in Table 1 and Table 2, respectively; all of the compounds contain the MBG attached to an amide-linked p-fluorobenzyl group. This makes all of these compounds analogs of a substructure of raltegravir, where only the oxadiazolyl substituent has been removed (FIG. 1). The omission of the oxadiazolyl substituent from the RCD compounds serves a dual purpose: 1) it greatly simplifies the synthesis of the desired compounds, and 2) differences in potency can be more directly attributed to changes in the MBG, rather than substituent effects. The MBGs employed in the RCD compounds cover a wide range of chelators including hydroxypyridinones (RCD-2, -3, -7), hydroxypyrones (RCD-4, -5, -6), catechols (RCD-8-, -9), p-dicarboxycatechols (RCD-10, -11), hydroxyquinolines (RCD-12, -13, 14), and several others. A total of 21 RCD derivatives were prepared, each with a unique MBG, and covering approximately ten chemically-distinct chelating motifs. In order to provide a suitable benchmark for comparison for these RCD compounds, the reported raltegravir derivative RCD-1 was prepared (FIG. 1). As with the other RCD compounds, RCD-1 is an abbreviated raltegravir derivative that lacks the oxadiazolyl substituent, but still shows good activity against HIV-1 IN (IC₅₀ value 60 nM against the strand transfer reaction of HIV-1 IN) (Pace P, et al., J. Med. Chem. 50(9):2225-2239 (2007)). The reduced activity of RCD-1 when compared to raltegravir is attributed to the loss of interactions between the omitted oxadiazolyl substituent and the surrounding active site residues, specifically Tyr143 of HIV-1 IN or Tyr212 in PFV (Metifiot M, et al., Biochemistry 49:3715-3722 (2010); Hare S, et al., Nature 464:232-237 (2010); Metifiot M et al., Viruses 2:1347-1366 (2010)).

HIV-1 IN Activity Screen

As described above, HIV-1 IN has two functions: 3′-processing (3P) and strand transfer (ST). Most HIV-1 IN inhibitors, including raltegravir, are targeted against the ST reaction of HIV-1 IN and hence are referred to as INSTIs. All 21 RCD compounds were screened for inhibitory activity against the 3P and ST reactions using published protocols (Metifiot M, et al., Biochemistry 49:3715-3722 (2010); Marchand C, Neamati N, & Pommier Y, Methods Enzymol 340:624-633 (2001)). Compounds were initially screened for activity at ˜100 μM, and those compounds that showed ST inhibition were then further examined to assess inhibition of viral replication. The results of the assays with the RCD compounds are listed in Table 1.

As expected, RCD-1 shows good activity against the ST reaction, with an IC₅₀ value of ˜1 μM. This is higher than the reported value of 60 nM (Pace P, et al., J. Med. Chem. 50(9):2225-2239 (2007)); however, under the assay conditions provided herein, raltegravir also produces a higher IC₅₀ value of ˜50 nM (Marinello J, et al., Biochemistry 47:9345-9354 (2008)). The difference in IC50 values results from differences in the assay. Some assays use preassembled HIV-1 IN on immobilized oligonucleotides (Pace P, et al., J. Med. Chem. 50(9):2225-2239 (2007)), whereas the assay provided herein uses ³²-P-end labeled oligonucleotides in solution and gel-based separation of the reaction products. RCD-1 also shows selectivity for the ST versus 3P reaction, consistent with previous findings (Marchand C, et al., Curr. Top. Med. Chem. 9:1016-1037 (2009)). Indeed, examination of the in vitro assay results immediately reveals that all of the RCD compounds, with a few exceptions (RCD-14, -16), are highly selective for ST versus 3P, suggesting a common mode of action.

Of the compounds prepared, four RCD inhibitors showed activity comparable or better than RCD-1. RCD-4, -5, -10, and -11 gave ST inhibition IC₅₀ values of 0.96, 0.55, 1.5, and 1.7 μM, respectively. Importantly, these compounds fall into only two distinct classes of MBG chelators: RCD-4 and RCD-5 contain hydroxypyrone chelators, while RCD-10 and RCD-11 contain p-dicarboxy catechol chelators. This clearly highlights the role of the MBG for inhibitor efficacy, whereby only two of at least ten distinct metal-binding groups resulted in good ST inhibition. Other compounds showed modest activity, including RCD-4S, RCD-4S², RCD-7, RCD-12, RCD-14, and RCD-16 with IC₅₀ values in the 4-20 μM range. Two compounds, RCD-6 and RCD-8, showed weaker activity with IC₅₀ values>40 μM. All of the remaining RCD compounds showed poor inhibition, with little or no activity at concentrations as high as 100 μM.

In addition to cell-free in vitro assays, eight RCD compounds were examined for inhibition of viral replication (Table 1) (Day J R, et al., J. Virol. Meth. 137(1):125-133 (2006)). Select RCD compounds, with different MBGs and including both active (RCD-1, -5, -10, -12, -14) and inactive (RCD-13, -17, -18) compounds, were examined Inhibition of viral replication by the selected RCDs in P4R5 cells was determined (Day J R, et al., J. Virol. Meth. 137(1):125-133 (2006)). Compounds with good ST activity were found to be the most effective at inhibiting P4R5 infection. RCD-1, -5, -10, -12, and -14, all of which have ST IC₅₀ values below 15 μM, were shown to have IC₅₀ values of <4.0 μM (Table 1). RCD-13, -17, and -18, which perform poorly in vitro (ST IC₅₀>100 μM), showed weak antiviral activity (IC₅₀>100 μM). Toxicity assays showed that most of the compounds tested in the viral replication assay showed little affect on P4R5 cells at a concentration of 10 μM (Hostetler K Y, et al., Antimicrob. Agents Chemother. 50:2857-2859 (2006)). Only RCD-12 and RCD-14 showed some toxicity at this concentration; therefore, follow up studies with these compounds or their derivatives will require greater consideration of their possible cytotoxicity. Overall, the cell-based infectivity assay was thus consistent with the in vitro ST activity, supporting the mechanism of action for the RCD compounds in HIV-1 IN inhibition.

Computational Docking Studies

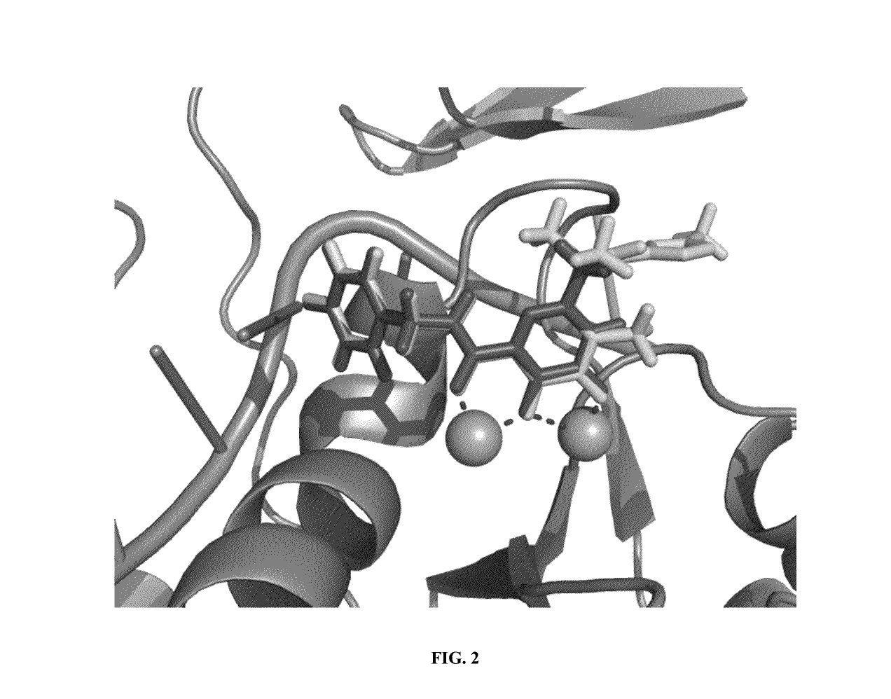

To elucidate the binding modes of the various RCD compounds, ligand-receptor docking studies were conducted. As previously described, the structure of the PFV IN in complex with raltegravir shows that the O,O,O donor triad binds to the active site Mg²⁺ ions, with the central oxygen atom acting as a bridge between the two metal centers. The coordinates for PFV IN (PDB: 3OYA) were used for computational docking of RCD compounds (Hare S, et al., Proceedings of the National Academy of Sciences 107(46):20057-20062 (2010); Krishnan L, et al., Proc Natl Acad Sci USA 107(36):15910-15915 (2010)). As a test of the docking procedure, raltegravir was docked into the PFV IN structure, resulting in a pose consistent with that seen in the crystal structure complex (RMSD 0.19 Å). RCD-1 was docked into PFV IN using the same procedure and gave a binding pose identical to that found for raltegravir (RMSD 0.25 Å, FIG. 2).

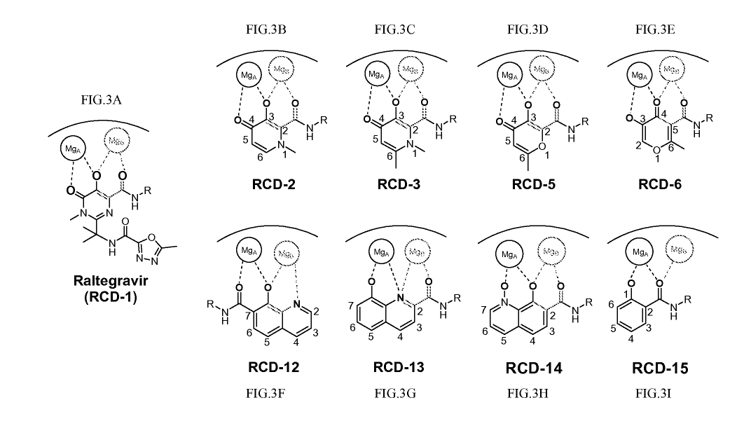

The O,O,O donor atom triad of raltegravir and RCD-1 bind to the Mg²⁺ ions forming 5- and 6-membered chelate rings (FIGS. 3A-3I). The hydroxyl oxygen and the amide-linked carbonyl oxygen together form the 6-membered ring while the same hydroxyl oxygen and the exocyclic carbonyl oxygen atom of the MBG make up the 5-membered ring. In both compounds, the deprotonated, anionic hydroxyl oxygen atom acts binds in a μ-bridging fashion between the two metal ions in the active site. The p-fluorobenzyl substituent of raltegravir and RCD-1 both rest in an identical pocket. It has been proposed that this pocket is formed by an induced fit mechanism upon displacement of an adenine residue (A17) from the nucleic acid substrate. The displacement of this nucleotide and the resulting pocket allow the p-fluorobenzyl group to interact with bases from the invariant CA dinucleotide, as well as residue Pro214 in the PFV intasome (equivalent to P145 in HIV-1 IN). The placement of this group is pivotal to the impairment of HIV-1 IN activity as it causes the viral DNA to be displaced from the active site (Hare S, et al., Nature 464:232-237 (2010)). This docking exercise with raltegravir and RCD-1 validated the assumption that the only difference in binding between these compounds is the omitted oxadiazolyl moiety, and that the omission of this group has little or no effect on the binding of the MBG or p-fluorobenzyl components of the INSTI.

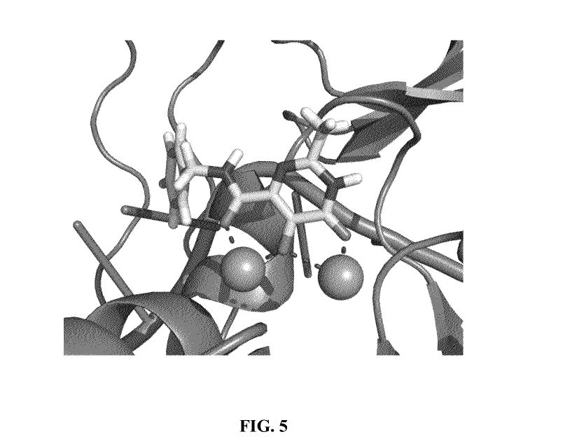

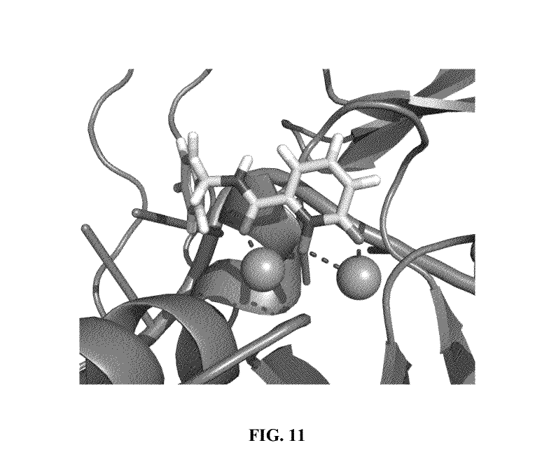

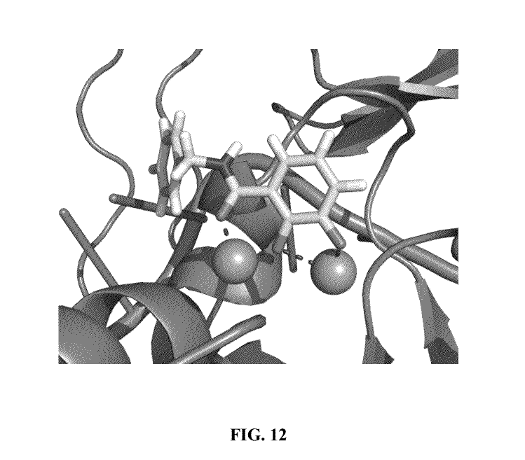

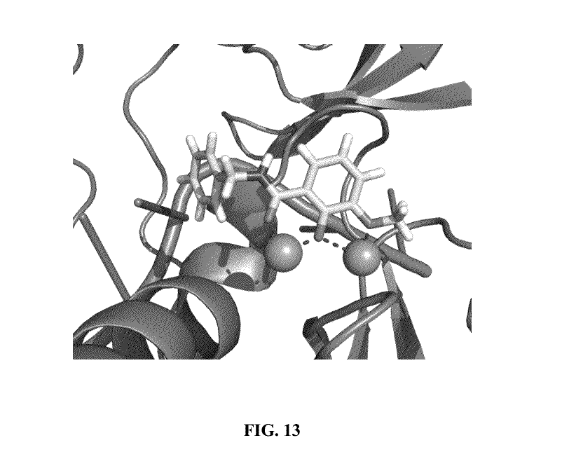

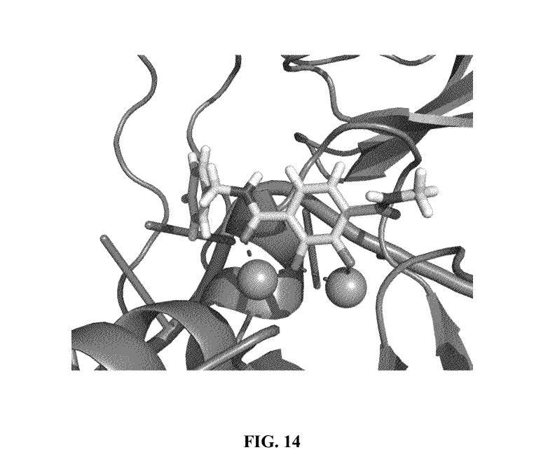

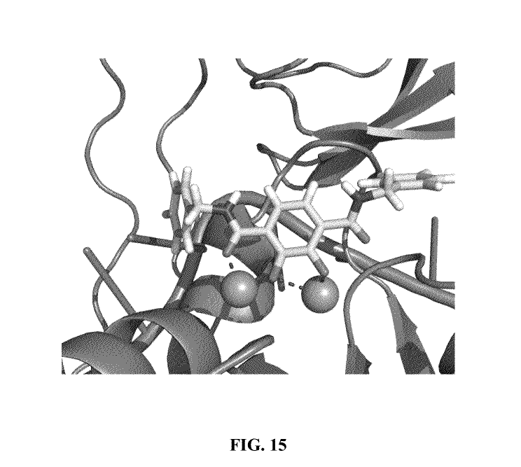

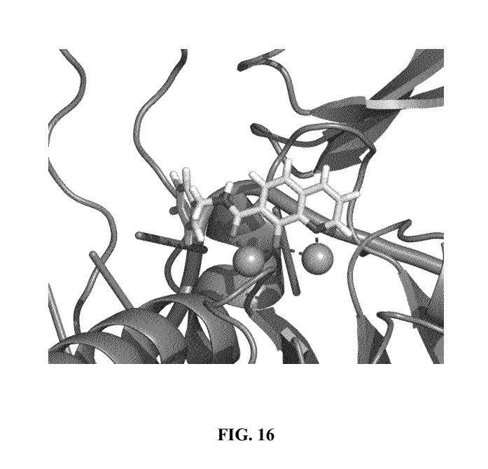

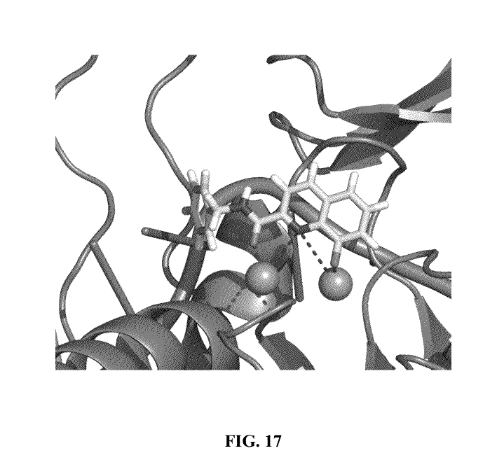

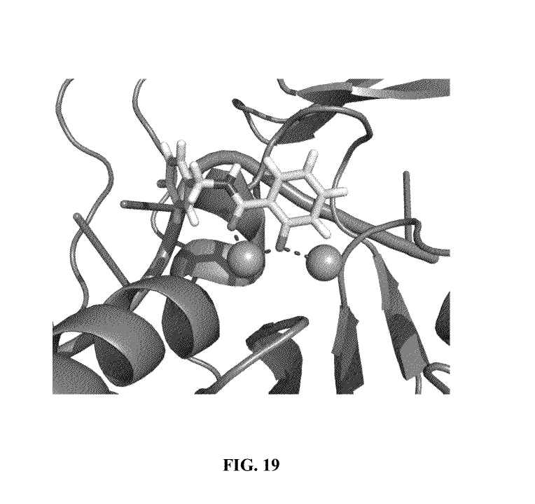

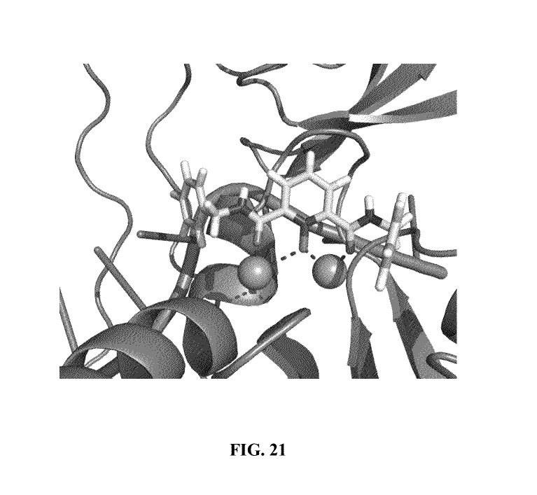

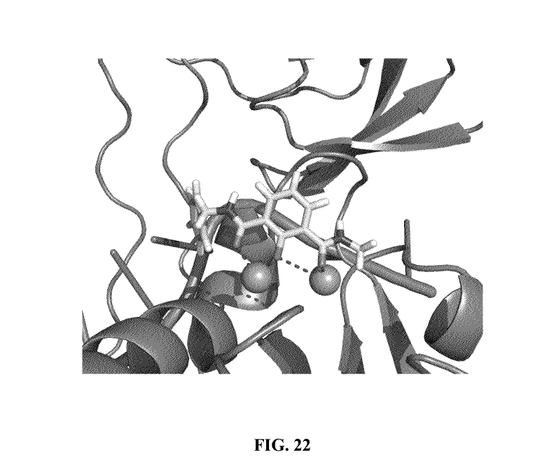

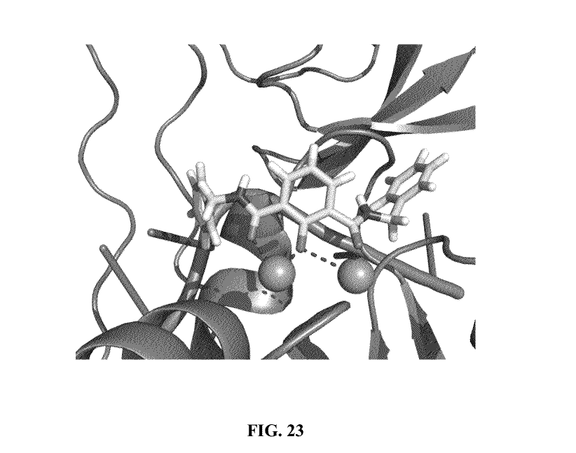

Satisfied with the validity of the docking procedure and parameters, the remaining RCD compounds were docked in a similar manner (FIGS. 5-23). Docking experiments showed that the other RCD compounds formed one of several chelate ring patterns (FIGS. 3A-3I): i) a 6-membered chelate ring with Mg_{B} and a 5-membered chelate ring with Mg_{A} (RCD-1 to RCD-12); ii) two 5-membered chelate rings (RCD-13); iii) two 6-membered chelate rings (RCD-14, -16, -17, -18, -19), or iv) only a single 6-membered chelate ring with Mg_{A} (RCD-15). In addition, for all RCD compounds, the p-fluorobenzyl substituent was bound in the same pocket as described for the raltegravir and RCD-1 compounds (vide supra). The findings and interpretation of these docking studies are discussed in detail in the section below.

Critical Features of MBGs

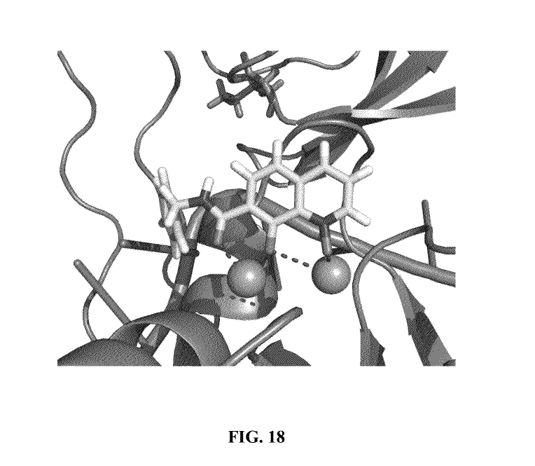

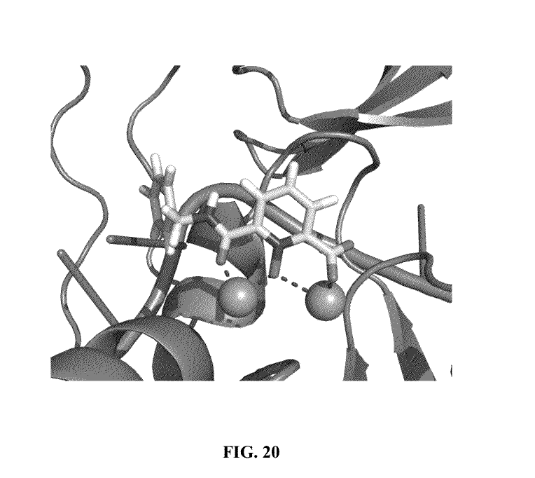

Inspection of the in vitro ST inhibition data, in conjunction with the computational docking experiments, reveals several interesting trends about the MBG requirements for this series of HIV-1 IN inhibitors. One feature that may be important is the size of the chelate rings formed upon binding of the inhibitor (FIGS. 3A-3I). Most of the active RCD compounds form a 5-membered chelate ring with Mg_{A} and a 6-membered chelate ring with Mg_{B} (RCD-1, -4, -5, -6, -7, -8, -10, -11, -12). Compounds that form two 5-membered chelate rings (RCD-13), two 6-membered chelate rings (RCD-17, -18, -19), or only a single chelate ring (RCD-15) were generally inactive. The preferred 5-,6-membered chelate ring binding arrangement found for most of the active RCD compounds is also formed by raltegravir (Hare S, et al., Nature 464:232-237 (2010)) and several other second-generation INSTIs (Hare S, et al., Proc Natl Acad Sci USA 107(46):20057-20062 (2010)), including L-870,810, GS9160, and MK0536 (FIG. 1). However, there are exceptions to the observed trends. For example, RCD-14 and RCD-16 both form two 6-membered chelate rings upon binding (FIG. 18, FIG. 20) and still exhibit moderate inhibition. These compounds both possess highly Lewis acidic (vide infra) N-oxide donors and form dianionic (2-) chelators upon metal binding, which should result in a stronger electrostatic attraction between the inhibitors and active site Mg²⁺ ions. These features may explain the enhanced activity of RCD-14 and RCD-16 despite what may be a sub-optimal coordination arrangement for this chemical scaffold.

Although the 5-,6-membered chelate ring appears to be favored by the RCD compounds and several other INSTIs, there are a number of examples in the literature indicating that other chelate ring motifs produce effective inhibitors. For example, dolutegravir reverses the size of the chelate rings, forming a 6-membered chelate ring with Mg_{A} and a 5-membered chelate ring with Mg_{B} (Hare S, et al., Mol Pharmacol In Press (2011)). However, the chelate ring motifs of other INSTIs differ more substantially. Structures of the second-generation inhibitors MK2048 and PICA (FIG. 1) bound to the PFV intrasome show that these compounds form two 6-membered and two 4-membered chelate rings, respectively (Hare S, et al., Proc Natl Acad Sci USA 107(46):20057-20062 (2010)). Elvitegravir utilizes yet another motif, forming a 6-,4-membered chelate ring arrangement (Hare S, et al., Nature 464:232-237 (2010)). Therefore, although the 5-,6-membered chelate ring arrangement appears to be most common among INSTIs, the numerous exceptions highlighted here clearly indicate that other productive binding modes are possible. Because of the intricate interplay between metal coordination and the positioning of the halogenated benzene group (Hare S, et al., Proc Natl Acad Sci USA 107(46):20057-20062 (2010)), it is likely that the metal chelate motif must be optimized in the context of different chemical scaffolds. Indeed, the RCD compounds also revealed an important trend concerning the relative positioning of the MBG to the p-fluorobenzyl backbone group.

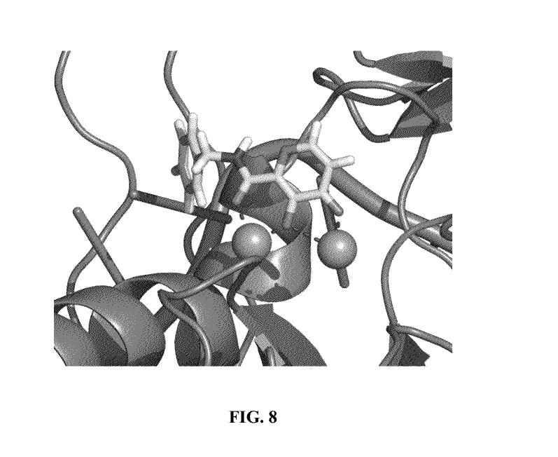

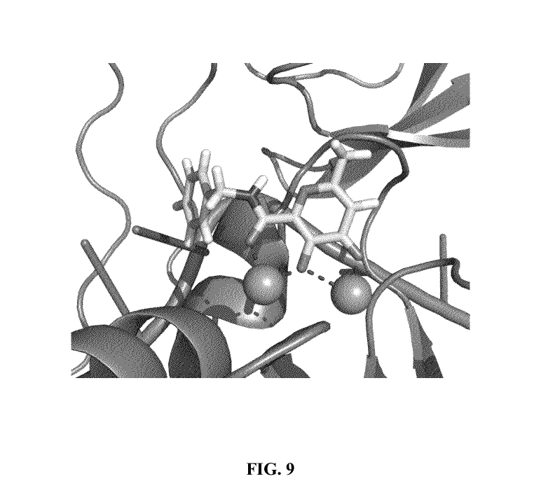

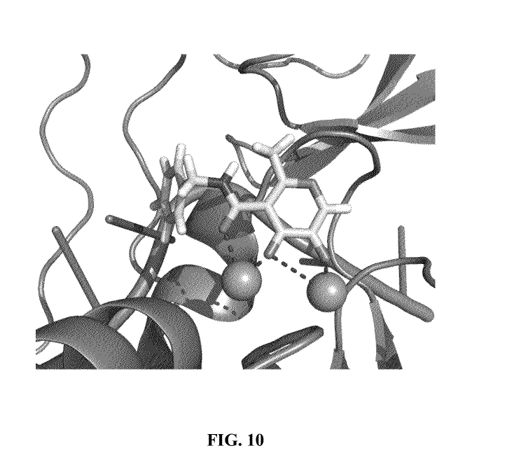

A second observation from the RCD inhibition data shows the importance of the relative orientation of the amide-linked p-fluorobenzyl group on the MBG. Comparison of RCD-5 to RCD-6 clearly shows how a change in the position of this substituent has a dramatic effect on activity. Both RCD-5 and RCD-6 contain the same hydroxypyrone MBG and can provide O,O,O donor atom triads to the active site metal ions (FIGS. 3A-3I). However, RCD-6 activity in vitro is found to be 100-fold less potent than RCD-5. Computational docking of RCD-5 and RCD-6 show that the molecules generally bind in a similar orientation, with little deviation (RMSD 0.30 Å) in the relative position of the p-fluorobenzyl group or in the scaffold of the MBG in the active site (FIG. 9, FIG. 10). However, the change in the point of attachment does affect the ordering of the oxygen atoms in the donor atom triad. The point of attachment of the p-fluorobenzyl group is the 2-position of the hydroxypyrone MBG ring in RCD-5, and the 5-position of the ring in RCD-6. As best illustrated in FIGS. 3A-3I, RCD-5 bridges the two active site metal-ions through the 3-hydroxyl oxygen atom. In contrast, for RCD-6 the bridging donor atom is the 4-carbonyl oxygen atom. This subtle change in the donor atom triad arrangement contributes to the notable loss in activity between RCD-5 and RCD-6. The anionic hydroxyl group is a stronger Lewis base donor than the neutral carbonyl and will serve as a stronger bridging donor atom between the Mg²⁺ ions. This argument is supported by the activity of RCD-4, which also contains a hydroxypyrone MBG with a p-fluorobenzyl group on the 2-position of the ring (it lacks a 6-methyl group found in RCD-5 and RCD-6, vide infra). Like RCD-5, RCD-4 presents the anionic hydroxyl atom as the bridging donor atom (FIG. 8) and similarly shows good ST inhibition (Table 1). Interestingly, essentially all of the lead INSTIs under investigation to date follow this motif, utilizing an anionic hydroxyl atom as the bridging atom (PICA is one notable exception) (Hare S, et al., Mol Pharmacol In Press (2011); Hare S, et al., Proc Natl Acad Sci USA 107(46):20057-20062 (2010); Hare S, et al., Nature 464:232-237 (2010)).

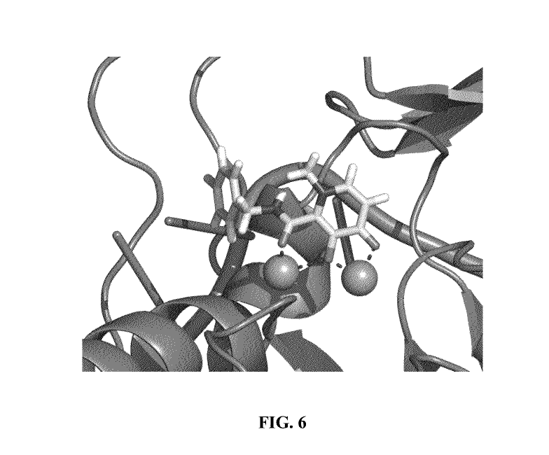

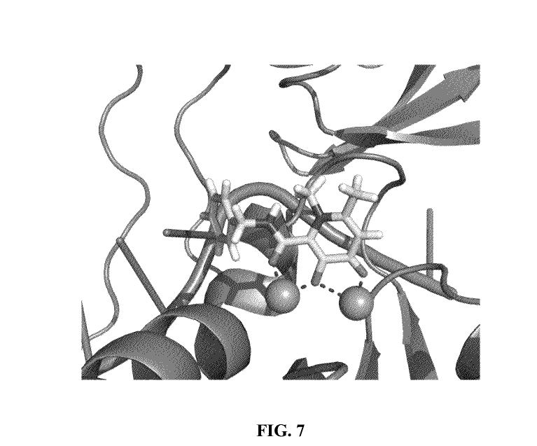

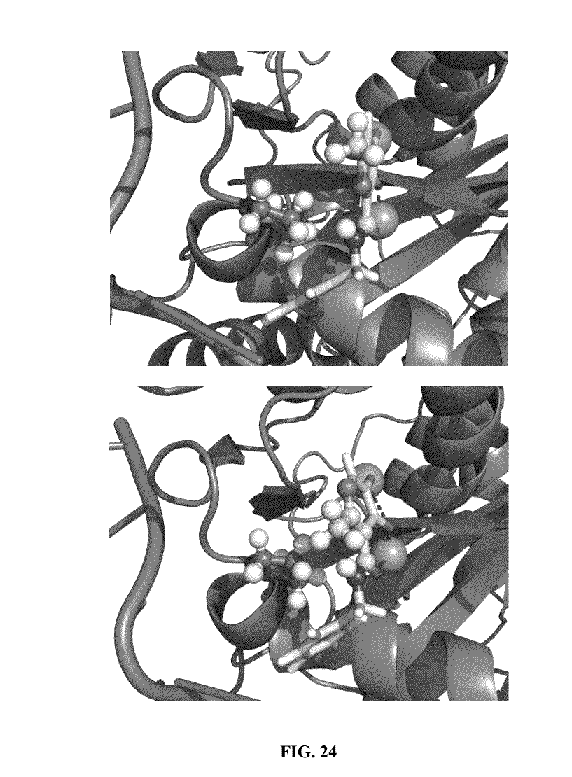

RCD-5 and RCD-6 both contain methyl groups at the 6-position of the MBG rings (FIGS. 3A-3I). In addition to the change in the arrangement of the donor atom triads discussed above, the difference in the position of the amide-linked p-fluorobenzyl group results in these methyl groups occupying different locations in the protein active site (FIG. 24). The orientation of the methyl group upon docking of RCD-5 in PFV IN does not result in any significant contacts with the protein. In contrast, the same methyl group, upon docking of RCD-6, results in a steric clash with Pro214 in the PFV IN active site (FIG. 24). Pro214 is one of the few conserved residues in the IN active site loop that is directly involved in separating the viral DNA strands, and both raltegravir and elvitegravir make intimate van der Waals interactions with this residue (Hare S, et al., Nature 464:232-237 (2010)). Therefore, the steric clash between Pro214 and the methyl group of RCD-6 also likely contributes to the loss of activity for this compound. The potential problems posed by the 6-methyl group in RCD-6 are further supported by the poor activity of hydroxypyridinones RCD-2 and RCD-3 (Table 1). The N-methyl group protruding from the MBGs in RCD-2 and RCD-3 is located in the same position as the 6-methyl group in RCD-6 (FIGS. 3A-3I). Indeed, docking experiments confirm a steric clash with Pro214 (FIG. 6, FIG. 7), as observed for RCD-6. Importantly, unlike RCD-6, RCD-2 and RCD-3 contain the preferred bridging hydroxyl group found in RCD-4 and RCD-5, suggesting that the steric problems posed by the methyl substituent may be the more significant factor when considering the loss in activity of RCD-2, -3, and -6. The comparisons between RCDs-2, -3, -4, -5, and -6 suggest that a combination of both the ordering of the donor triad as well as steric interactions can have a drastic effect on the potency of these inhibitors.

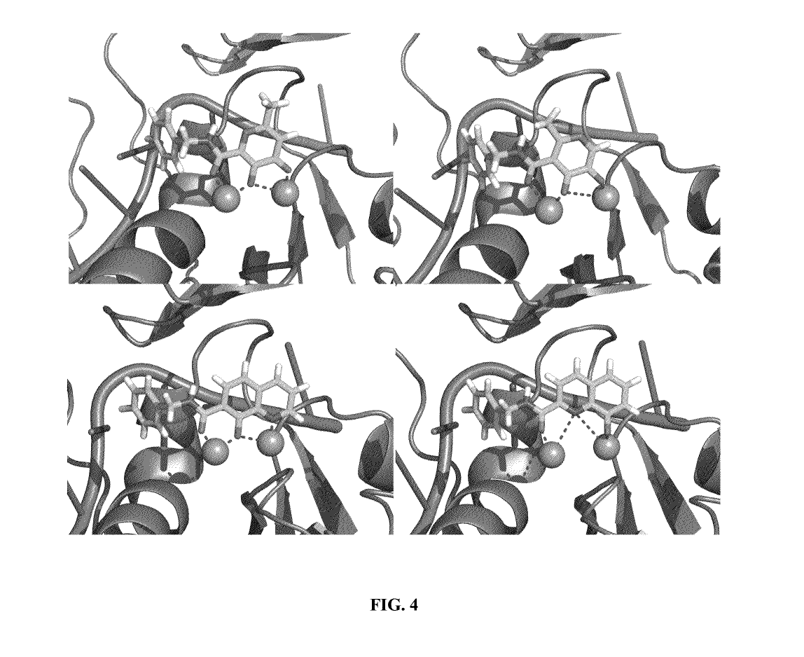

The dependence on the position of the amide p-fluorobenzyl substituent is also observed when comparing RCD-12 and RCD-13, both of which contain an 8-hydroxyquinoline MBG with identical O,O,N donor atom sets. RCD-13, which contains the amide group at the 2-position, shows minimal (<30%) inhibition at ˜100 μM while RCD-12, which has the amide substituent attached at the 7-position, shows good activity with an IC₅₀ value of ˜14 μM. As with RCD-5 and RCD-6, RCD-12 and RCD-13 have the same molecular formula, overall composition, and MBG that provides an identical donor atom set (one hydroxyl oxygen atom, one amide oxygen atom, and one quinoline nitrogen atom). However, the position of the p-fluorobenzyl affects the overall arrangement of the donor atoms upon binding to the active site metal ions. As confirmed by docking studies (FIG. 4), the position of the p-fluorobenzyl amide substituent in RCD-12 versus RCD-13 results in a significant change in the arrangement of the donor atom triad for these two compounds. For RCD-13 the donor set will be arranged as O,N,O while for RCD-12 the arrangement will be O,O,N (FIG. 4), resulting in the donor atom arrangement for RCD-12 forming 6-membered and 5-membered chelate rings, with a bridging hydroxyl atom. The same arrangement is found in raltegravir and the other most active RCD compounds identified here. In contrast, when the p-fluorobenzyl amide group is attached to the 2-position of the scaffold as in RCD-13, the chelator is forced to adopt two 5-membered chelate rings, with the quinoline nitrogen atom serving as the bridging ligand. Such endocyclic nitrogen atoms do not readily engage in bridging modes of metal ion coordination (Kaes C, Katz A, & Hosseini M W, Chem. Rev. 100(10):3553-3590 (2000)). Furthermore, the quinoline nitrogen atom is positioned too far from the Mg²⁺ ions (>3.7 Å) to form strong interactions. Despite the similar arrangement of the donor triad in RCD-12, this compound is still less potent than RCD-4 and RCD-5, which is likely due to the preference of the hard Mg²⁺ ions for the harder oxygen atom donor set found in the hydroxypyrone compounds. Hard Lewis base donors like anionic oxygen atoms are classically characterized by their small size, high charge state, and weak polarizability (Ho T-L, Chem. Rev. 75(1):1-20 (1975)). Comparing these compounds clearly shows that having a heteroatom triad is not sufficient for good inhibition, but rather the correct or optimal atom arrangement of the triads is also essential along with the optimal matching of the Lewis acid character of the donor atoms.

The comparison between RCD-4/-5 and RCD-12 highlights a third trend related to the nature of the MBG donor atoms. The preference for certain donor atoms was explored by converting the O,O,O donor RCD-4 to two different sulfur analogs. As stated above, the catalytic Mg²⁺ ions are hard Lewis acids and hence should bind more tightly to harder Lewis base donor atoms. The introduction of softer, more polarizable Lewis base sulfur atoms to the donor triad were expected to lower the efficacy of the compounds. Isostructural hydroxypyrothione analogs, termed RCD-4S and RCD-4S² (Table 1) provide O,O,S and S,O,S donor atom sets, respectively. Both RCD-4S and RCD-4S² show a significant loss in activity when compared to RCD-4. The weaker ST inhibition by RCD-4S and RCD-4S² is likely due to a hard-soft mismatch between the hard Lewis acid Mg²⁺ ions and the soft Lewis base sulfur donor atoms. This conclusion is consistent with the improved performance of sulfur compounds like RCD-4S² against metalloenzymes that are dependent on the softer Lewis acid Zn²⁺ ion, such as the anthrax lethal factor (LF). In the case of anthrax LF, RCD-4S² is a better inhibitor than RCD-4 (Agrawal A, et al., J. Med. Chem. 52:1063-1074 (2009); Lewis J A, et al., Chem Med Chem 1(7):694-697 (2006)), precisely the opposite of what is observed for HIV-1 IN. Hence, the selection of the donor atoms with the appropriate Lewis acid character is important for obtaining optimal inhibition of HIV-1 IN.

Novel MBG Scaffolds

In this study, two, novel MBG types that appear to be promising new scaffolds for the development of HIV-1 IN inhibitors have been identified. The first MBG is the hydroxypyrone group found in RCD-4 and RCD-5, both of which show good in vitro activity and RCD-5 also displayed good cell-based activity. The hydroxypyrone MBGs found in these compounds derive from the FDA-approved food additive maltol (3-hydroxy-2-methyl-4H-pyran-4-one) for which there has been extensive chemistry developed that should facilitate the preparation of even more potent inhibitors based on this scaffold (Finnegan M M, Rettig S J, & Orvig S J, J. Am. Chem. Soc. 108:5033-5035 (1986); Schugar H, et al., Angew Chem Int Edit 46(10):1716-1718 (2007); Puerta D T et al., J. Am. Chem. Soc. 127:14148-14149 (2005)). The second class of compounds that warrants additional investigation are those based on the p-dicarboxy catechol MBGs (RCD-10 and RCD-11). Four compounds were examined that are nominally based on a catechol MBG: RCD-8, RCD-9, RCD-10, and RCD-11. RCD-8 contains a catecholamide MBG and shows modest ST inhibition with an IC₅₀ value of 39 μM. RCD-9 shows a complete loss of activity due to methylation of one of the phenol groups resulting in a reduced donor ability, while addition of a second carboxyamide group in RCD-10 and RCD-11 produces a significant improvement (>20-fold) in activity with IC₅₀ values<2 μM. One possible explanation for the improved activity of RCD-10 and RCD-11 over RCD-8 would be additional interactions between the protein active site and the added carboxyamide substituents; however, RCD-10 and RCD-11 have very different substituents (methyl versus p-fluorobenzyl, Table 1), but essentially identical ST inhibition IC₅₀ values (1.5 and 1.7 μM, respectively). With this observation in mind, the origin of the improved activity of RCD-10 and RCD-11 relative to RCD-8 is attributed to the reduced pKₐ of the MBG. In order to obtain optimal binding to the Mg²⁺ ions, the MBGs should be deprotonated upon metal binding. Catechol is a strong, hard Lewis donor, but it is also very basic (pKₐ₁=9.2, pKₐ₂˜13) (Gorden A E V et al., Chem. Rev. 103(11):4207-4282 (2003)) making deprotonation under physiological conditions more difficult. Addition of electron withdrawing groups, such as the carboxyamide groups used in the RCD compounds described here, are known to significantly reduce the pKₐ of the catechol ligand (Gorden A E V et al., Chem. Rev. 103(11):4207-4282 (2003)). Therefore, the addition of a second such carboxyamide group will result in an inhibitor that more readily achieves deprotontion of both phenolic groups in the catechol ligand, resulting in a dianionic (2-) ligand and a strong electrostatic attraction between the MBG and the active site metal ions.

While numerous inhibitors have been prepared and studied (Pommier Y, Johnson A A, & Marchand C, Nat. Rev. Drug Dis. 4(3):236-248 (2005); Marchand C, et al., Curr. Top. Med. Chem. 9:1016-1037 (2009); Serrao E et al., Retrovirology 6:25-39 (2009)), few or none have systematically dissected and evaluated the contribution and structure-activity relationship around the MBGs in these compounds (Bacchi A, et al., J. Med. Chem.:ASAP contents (2011)). By preparing and evaluating the RCD compounds reported here, a number of important features of the MBG for use in INSTIs have been identified, including: a) the heteroatom triad should consist of hard Lewis base donor atoms to match the hard Lewis acid character of the active site Mg²⁺ ions; b) the triad should possess a geometry that results in the formation of optimal chelate ring sizes (for RCDs this appears to be adjacent 5-(Mg_{A}) and 6-(Mg_{B}) membered rings); and c) the hardest, anionic donor atom should be located in the middle of the triad to provide a sufficiently electron-donating ligand in the μ-bridging position between the metal ions (Kirschberg T & Parrish J, Curr. Opin. Drug Discov. Dev. 10:460-472 (2007)). These experiments also lead to the identification of at least two new and distinct MBGs, hydroxypyrones (RCD-4 and RCD-5) and p-dicarboxy catechols (RCD-10 and RCD-11) that may prove to be promising scaffolds for next-generation HIV-1 IN inhibitors. Overall, these studies provide direct evidence that subtle variations in the MBG can substantially affect the activity of an HIV-1 IN inhibitor, and suggests that rational approaches to strengthening metal-ligand interactions can produce potent inhibitors to help mitigate the need for other active site interactions and hence overcome rising resistance against raltegravir.

Materials and Methods

All RCD compounds were prepared using standard synthetic methods, similar to those previously described (Agrawal A, et al., J. Med. Chem. 52:1063-1074 (2009)). Computational docking was preformed using the Glide software package (Glide v5.5; Schrodinger, Inc.). Enzyme and cell-based assays were performed as previously described (Metifiot M, et al., Biochemistry 49:3715-3722 (2010); Marchand C, Neamati N, & Pommier Y, Methods Enzymol 340:624-633 (2001); Day J R, et al., J. Virol. Meth. 137(1):125-133 (2006)). Complete synthetic and experimental details are provided herein.

Unless otherwise noted, starting materials were purchased from commercial suppliers (Sigma-Aldrich, ChemBridge, Acros Organics, TCI America) and were used without further purification. Chromatography was preformed using a CombiFlash Rf 200 from TeledyneISCO. ¹H NMR spectra were recorded on one of several Varian FT-NMR spectrometers, property of the Department of Chemistry and Biochemistry, University of California San Diego. Mass spectrometry was performed at the Small Molecule Spectrometry Facility in the Department of Chemistry and Biochemistry, University of California San Diego. Compounds RCD-2, RCD-3, RCD-4, RCD-4S, RCD-4S², RCD-5, RCD-7, 4, and 12 were all synthesized as previously described (Agrawal, A.; De Oliveira, C. A. F; et al. J. Med. Chem. 2009, 52, 1063; Agrawal, A.; Romero-Pereze, D.; et al. Chem Med Chem 2008, 3, 812; Yan, Y. et al. Org. Lett. 2007, 9, 2517; Yan, Y.; Miller, M.; et al. Bioorg. Med. Chem. Lett. 2009, 19, 1970; Karpishin, T. B. et al. J. Am. Chem. Soc. 1993, 115, 182; K. Raymond, J. Xu, in United States Patent and Trademark Office (Ed.: U. S. P. T. Office), The Regents of the University of California (Oakland, Calif.) U.S. Pat. No. 5,892,029, U.S., 1999.).

Synthetic Chemistry

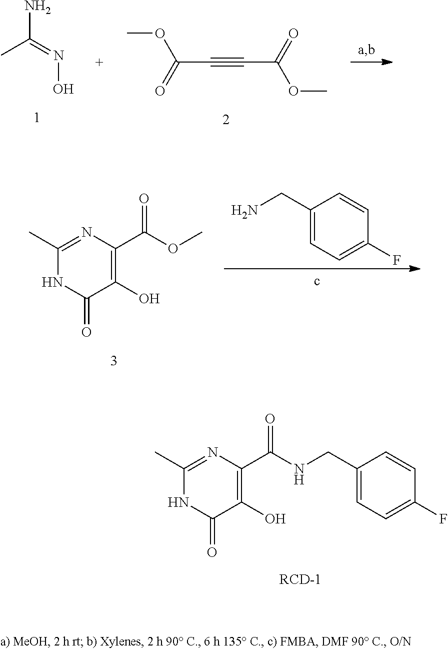

### Methyl 5-hydroxy-2-methyl-6-oxo-1,6-dihydropyrimidine-4-carboxylate (3)

The synthesis of this compound was adapted from a literature procedure (Belyk, K. M.; Morrison, H. G.; Jones, P.; Summa, V. Preparation of N-(4-fluorobenzyl)-5-hydroxy-1-methyl-2-(1-methyl-1-{[(5-methyl-1,3,4-oxadiazol-2-yl)carbonyl]amino}ethyl)-6-oxo-1,6-dihydropyrimidine-4-carboxamide potassium salts as HIV integrase inhibitors. PCT Int. Appl. WO/2006/060712, 2006). To a solution of (E)-N′-hydroxyacetimidamide (1) (500 mg, 6.75 mmol) in 8 mL of MeOH, was added 900 μL dimethyl but-2-ynedioate (2). After 1 h at room temperature, 6 mL of xylenes was added and the MeOH was removed. The solution was then refluxed at 135° C. for 16 h. The solution was cooled to 60° C., and 3 mL of MeOH was added with stirring. After 30 minutes, 8 mL of methyl t-butyl ether (MTBE) was added dropwise and the solution was kept at 0° C. for 16 h. The black precipitate was filtered off and rinsed with cold 10% MeOH/MTBE. Yield=44%. ¹H NMR (400 MHz, CDCl₃, 25° C.): δ=2.51 (s, 3H), 4.04 (s, 3H), 10.73 (br, 1H; NH). ESI-MS(+) m/z 184.9 [M+H]⁺.

### N-(4-Fluorobenzyl)-5-hydroxy-2-methyl-6-oxo-1,6-dihydropyrimidine-4-carboxamide (RCD-1)

The synthesis of this compound was adapted from literature procedure (Summa, V.; Petrocchi, A.; Matassa, V. G.; et al. J. Med. Chem. 2006, 49, 6646). 5,6-Dihydroxy-2-methyl-pyrimidine-4-carboxylic acid methyl ester (1c) (100 mg, 0.54 mmol) and (4-fluorophenyl)methanamine (FPMA, 124 μL, 1.1 mmol) were combined in 3 mL DMF and refluxed at 90° C. for 16 h. The reaction was then cooled to room temperature, and 1M HCl was added until precipitate formed. The solution was cooled further to 0° C. for 30 minutes. The precipitate was filtered and rinsed with ether. A dark brown solid obtained. Yield=38%. ¹H NMR (400 MHz, DMSO-d₆, 25° C.): δ=2.23 (s, 3H), 4.42 (d, J=4.0 Hz, 2H), 7.13 (t, J=8.0 Hz, 2H; ArH), 7.35 (t, J=6.0 Hz, 2H; ArH), 9.33 (brt, J=8.0 Hz, 1H; NH). ESI-MS(+) m/z 278.0 [M+H]⁺. Anal. Calcd for C₁₃H₁₂FN₃O₃: C, 56.32; H, 4.36; N, 15.16. Found: C, 56.31; H, 4.38; N, 15.11.

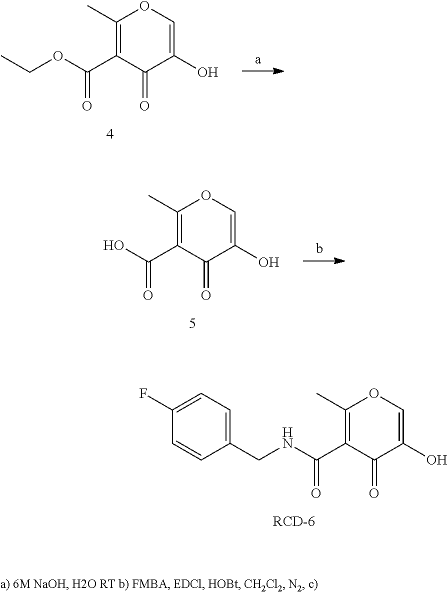 [SMILES: CCOC(=O)c1c(C)occ(O)c1=O ; Cc1occ(O)c(=O)c1C(=O)O ; Cc1occ(O)c(=O)c1C(=O)NCc1ccc(F)cc1]

### 5-Hydroxy-2-methyl-4-oxo-4H-pyran-3-carboxylic acid (5)

To a solution of 4 (250 mg, 1.26 mmol) in 5 mL of H₂O was added, 3 mL of a 6M NaOH solution. The mixture was stirred for 3 h at room temperature under nitrogen. The reaction was evaporated under vacuum and the product (5) was extracted with CH₂Cl₂ and washed with 6M HCl. The organic phase was dried over anhydrous MgSO₄ and concentrated to a yellow solid (150 mg, 0.88 mmol). Yield=70%. ¹H NMR (400 MHz, DMSO-d₆, 25° C.): δ=2.29 (s, 2H; CH₃), 8.05 (s, 1H; ArH), 9.36 (s, 1H; ArOH). ESI-MS(−) m/z 169.22 [M−H]⁻.

### 4-Fluorobenzyl 5-hydroxy-2-methyl-4-oxo-4H-pyran-3-carboxylate (RCD-6)

To a solution of 5 (60 mg, 0.35 mmol) in 10 mL of dry CH₂Cl₂ was added 1-ethyl-3-(3-dimethylaminopropyl) carbodiimide (EDCI, 81 mg, 0.42 mmol), hydroxybenzotriazole (HOBt, 57 mg, 0.42 mmol), and FPMA (48 μL, 0.42 mmol). The mixture was stirred overnight at room temperature under nitrogen and extracted with 1M HCl and CH₂Cl₂. The organic phase was dried over anhydrous MgSO₄, filtered, and concentrated to a yellow solid. The crude solid was purified via silica column chromatography (0-5% MeOH/CH₂Cl₂) to obtain the product as a yellow solid (28 mg, 0.10 mmol). Yield=29%. ¹H NMR (500 MHz, DMSO-d₆, 25° C.): δ=2.31 (s, 3H; CH₃), 5.64 (d, J=2.8 Hz, 2H; CH₂), 7.08 (dd, J=9.2, 2.8 Hz, 2H; ArH), 7.35-7.37 (m, 2H; ArH), 7.99 (s, 1H; ArH), 7.20 (brt, 1H; CONHCH₂). ESI-MS(−) m/z 276.25 [M−H]⁻. Anal. Calcd for C₁₄H₁₂FNO₄: C, 60.65; H, 4.36; N, 5.05. Found: C, 61.04; H, 4.76; N, 5.13.

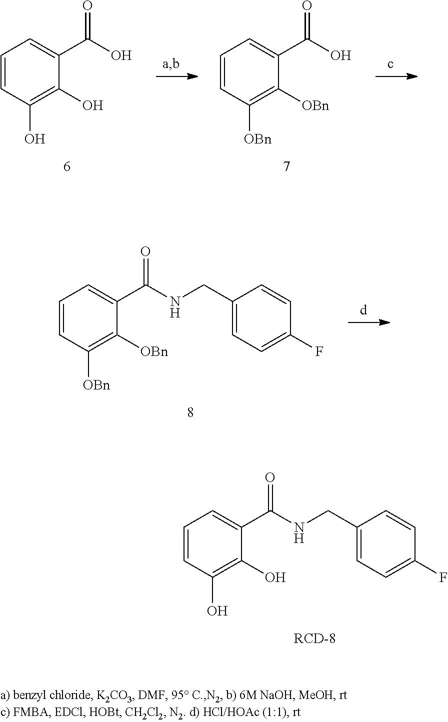 [SMILES: O=C(O)c1cccc(O)c1O ; O=C(O)c1cccc(OCc2ccccc2)c1OCc1ccccc1 ; O=C(NCc1ccc(F)cc1)c1cccc(OCc2ccccc2)c1OCc1ccccc1 ; O=C(NCc1ccc(F)cc1)c1cccc(O)c1O]

### 2,3-Bis(benzyloxy)benzoic acid (7)

To a solution of dihydroxybenzoic acid (6) (500 mg, 3.24 mmol) in 30 mL of DMF, benzyl chloride (1.33 mL, 11.6 mmol) and K₂CO₃ (1.71 g, 12.4 mmol) was added. The resulting mixture was then heated to reflux at 120° C. under nitrogen and stirred overnight. The reaction mixture was filtered and the filtrate was evaporated under vacuum to obtain a brown oil. The crude oil was purified via a silica plug using CH₂Cl₂ as eluant. Evaporation of the solvent gave a clear oil (1.36 g, 3.12 mmol). Yield=96%. To a solution of the oil (1.32 g, 3.11 mmol) in 10 mL of MeOH, was added 6 mL of 6M NaOH. The mixture was stirred overnight at room temperature under nitrogen. The solvent was evaporated under vacuum and the product (7) was extracted into CH₂Cl₂ and washed with 6M HCl. The organic phase was collected, dried over anhydrous MgSO₄, and evaporated under vacuum to give a white solid. Yield=99%. ¹H NMR (400 MHz, CDCl₃, 25° C.): δ=5.20 (s, 2H; CH₂), 5.27 (s, 2H; CH₂), 7.17 (t, J=8.0 Hz, 1H; ArH), 7.27-7.50 (m, 10H; ArH), 7.73 (dd, J=7.6, 1.6 Hz, 1H; ArH). ESI-MS(−) m/z 332.92 [M−H]⁻.

### 2,3-Bis(benzyloxy)-N-(4-fluorobenzyl)benzamide (8)

To a solution of 7 (500 mg, 1.49 mmol) in 15 mL of dry CH₂Cl₂, was added EDCI (343 mg, 1.79 mmol), HOBt (242 mg, 1.79 mmol), and FPMA (204 μL, 1.79 mmol). The mixture was stirred overnight at room temperature under nitrogen. The reaction was extracted with CH₂Cl₂ and washed with 1M HCl. The organic phase was collected, dried over anhydrous MgSO₄, and concentrated under vacuum to obtain a brown oil. The oil was purified via silica column chromatography with 0-1% MeOH/CH₂Cl₂ as eluant to yield a white solid. Yield=58%. ¹H NMR (400 MHz, CDCl₃, 25° C.): δ=4.42 (d, J=5.6 Hz, 2H; NHCH₂), 4.99 (s, 2H; CH₂), 5.09 (s, 2H; CH₂), 6.93 (t, J=8.6 Hz, 2H; ArH), 7.14-7.18 (m, 5H; ArH), 7.23 (d, J=7.0 Hz, 2H; ArH), 7.27 (d, J=7.6 Hz, 1H; ArH), 7.31 (t, J=7.4 Hz, 2H; ArH), 7.37-7.43 (m, 2H; ArH), 7.47 (d, J=7.6 Hz, 2H; ArH), 7.81 (dd, J=6.0, 3.2 Hz, 1H; ArH), 8.42 (t, J=5.4 Hz, 1H; CONHCH₂). ESI-MS(+) m/z 441.91 [M+H]⁺, 464.01 [M+Na]⁺.

### N-(4-Fluorobenzyl)-2,3-dihydroxybenzamide (RCD-8)

Compound 8 (372 mg, 0.84 mmol) was stirred in 25 mL of a 1:1 solution of HCl:HOAc at room temperature for 5 d to obtain a turbid mixture. The solution was evaporated to dryness and the resulting residue was co-evaporated with 3×5 mL of MeOH and the resulting solid was dried overnight in a vacuum oven to yield the product as a white solid (186 mg, 0.71 mmol). Yield=85%. ¹H NMR (400 MHz, DMSO-d₆, 25° C.): δ=4.45 (d, J=6.0 Hz, 2H; NHCH₂), 6.65 (t, J=8.0 Hz, 1H; ArH), 6.89 (d, J=7.6 Hz, 1H; ArH), 7.12 (t, J=8.8, Hz, 2H; ArH), 7.29 (d, J=8.4 Hz, 1H; ArH), 7.33 (dd, J=8.4, 2.8 Hz, 2H; ArH), 9.31 (t, J=6.0 Hz, 1H; CONHCH₂). APCI-MS(+) m/z 262.11 [M+H]⁺. Anal. Calcd for C₁₄H₁₂FNO₃.0.5H₂O: C, 62.22; H, 4.85; N, 5.18. Found: C, 62.36; H, 5.09; N, 5.23.

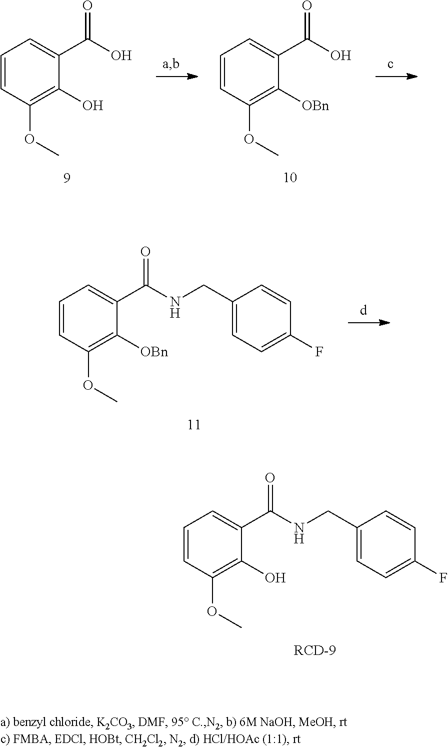 [SMILES: COc1cccc(C(=O)O)c1O ; COc1cccc(C(=O)O)c1OCc1ccccc1 ; COc1cccc(C(=O)NCc2ccc(F)cc2)c1OCc1ccccc1 ; COc1cccc(C(=O)NCc2ccc(F)cc2)c1O]

### 2-(Benzyloxy)-3-methoxybenzoic acid (10)

To a solution of 3-methoxysalicylic acid (9, 500 mg, 2.97 mmol) in 10 mL of DMF was added benzyl chloride (880 μL, 7.63 mmol) and K₂CO₃ (1.16 g, 8.41 mmol). The resulting mixture was heated to reflux at 120° C. under nitrogen and stirred overnight. The reaction was vacuum filtered and the filtrate was concentrated to a dark brown oil. The oil was purified via a silica plug using CH₂Cl₂ as an eluant, after which removal of solvent under vacuum gave an off-white oil (763 mg, 2.19 mmol). Yield=74%. To a solution of the oil (763 mg, 2.19 mmol) in 5 mL of MeOH was added 3 mL of 6M NaOH. The mixture was stirred overnight at room temperature under nitrogen. The reaction was evaporated under vacuum and the product was extracted with CH₂Cl₂ and washed with 6M HCl. The organic phase was collected, dried over anhydrous MgSO₄, and concentrated under vacuum to an off-white solid (566 mg, 2.19 mmol). Yield=99%. ¹H NMR (300 MHz, CDCl₃, 25° C.): δ=3.97 (s, 3H; OCH₃), 5.27 (s, 2H; CH₂), 7.19 (d, J=3.6 Hz, 1H; ArH), 7.36-7.41 (m, 5H; ArH), 7.43 (d, J=2.1 Hz, 1H; ArH), 7.68 (dd, J=6.3, 3.0 Hz, 1H; ArH). ESI-MS(+) m/z 259.11 [M+H]⁺, 276.10 [M+NH₄]⁺.

### 2,3-Bis(benzyloxy)-N-(4-fluorobenzyl)benzamide (11)

To a solution of 10 (566 mg, 2.19 mmol) in 15 mL of dry CH₂Cl₂ was added EDCI (504 mg, 2.63 mmol), HOBt (335 mg, 2.63 mmol), and FPMA (301 μL, 2.63 mmol). The mixture was stirred overnight at room temperature under nitrogen, after which the solution was extracted with CH₂Cl₂ and washed with 1M HCl. The organic phase was collected, dried over anhydrous MgSO₄, and concentrated under vacuum to give a yellow oil. The oil was purified via silica column chromatography using 0-2% MeOH/CH₂Cl₂ as eluant, after which removal of solvent under vacuum gave an off-white solid (383 mg, 1.05 mmol). Yield=48%. ¹H NMR (400 MHz, CDCl₃-d₁, 25° C.): δ=3.92 (s, 3H; OCH₃), 4.41 (d, J=5.6 Hz, 2H; NHCH₂), 4.99 (s, 2H; CH₂), 6.91 (t, J=8.8 Hz, 2H; ArH), 7.07 (dd, J=8.0, 1.6 Hz, 1H; ArH), 7.11 (dd, J=8.4, 5.2 Hz, 2H; ArH), 7.16 (t, J=8.2 Hz, 1H; ArH), 7.22 (dd, J=7.2, 1.6 Hz, 2H; ArH), 7.29-7.37 (m, 3H; ArH), 7.74 (dd, J=7.6, 1.6, Hz, 1H; ArH), 8.31 (brs, 1H; CONHCH₂). ESI-MS(+) m/z 366.27 [M+H]⁺, 388.25 [M+Na]⁺.

### N-(4-Fluorobenzyl)-2-hydroxy-3-methoxybenzamide (RCD-9)

Compound 11 (300 mg, 0.82 mmol), was stirred in 10 mL of a 1:1 solution of HCl:HOAc at room temperature for 5 d to obtain a turbid mixture. The solution was evaporated to dryness and the resulting residue was co-evaporated with 3×5 mL of MeOH and the resulting solid was dried overnight in a vacuum oven to yield the product as a white solid (163 mg, 0.59 mmol). Yield=72%. ¹H NMR (500 MHz, DMSO-d₆, 25° C.): δ=3.74 (s, 3H; OCH₃), 4.43 (d, J=6.3 Hz, 2H; NHCH₂), 6.78 (t, J=8.0 Hz, 1H; ArH), 7.07 (d, J=7.4 Hz, 1H; ArH), 7.11 (t, J=8.9, Hz, 2H; ArH), 7.31 (dd, J=8.6, 3.4 Hz, 2H; ArH), 7.41 (dd, J=8.0, 1.1 Hz, 2H; ArH), 9.32 (t, J=6.0 Hz, 1H; CONHCH₂). ¹³C NMR (125 MHz, DMSO-d₆, 25° C.): 42.1 (CH₂), 56.2 (OCH₃), 115.5 (ArC), 115.6 (ArC), 115.9 (ArC), 118.4 (ArC), 119.1 (ArC), 129.7 (ArC), 129.8 (ArC), 135.5 (ArC), 148.9 (ArC), 151.2 (ArC), 169.8 (C═O). ESI-MS(+) m/z 276.20 [M+H]⁺. Anal. Calcd for C₁₅H₁₄FNO₃: C, 65.45; H, 5.13; N, 5.09. Found: C, 65.76; H, 5.51; N, 5.12.

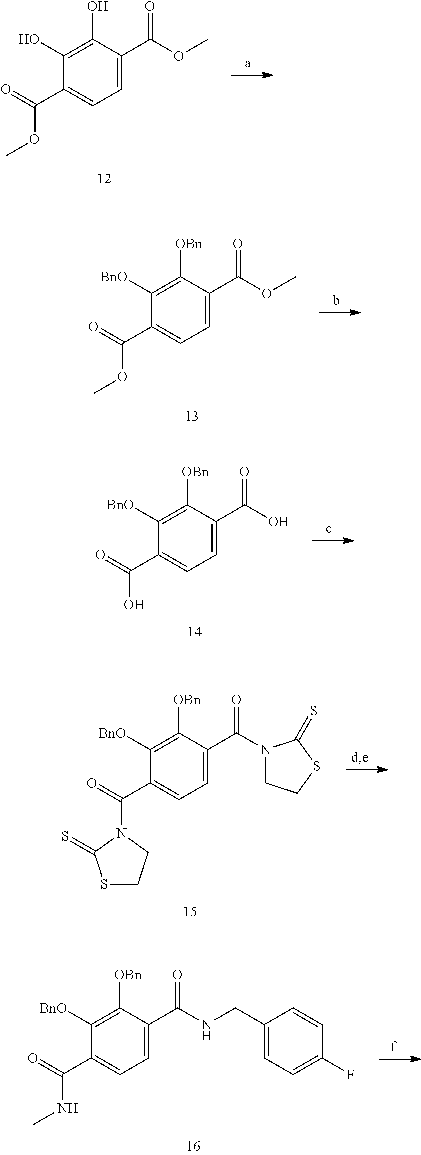 [SMILES: COC(=O)c1ccc(C(=O)OC)c(O)c1O ; COC(=O)c1ccc(C(=O)OC)c(OCc2ccccc2)c1OCc1ccccc1 ; O=C(O)c1ccc(C(=O)O)c(OCc2ccccc2)c1OCc1ccccc1 ; O=C(c1ccc(C(=O)N2CCSC2=S)c(OCc2ccccc2)c1OCc1ccccc1)N1CCSC1=S ; CNC(=O)c1ccc(C(=O)NCc2ccc(F)cc2)c(OCc2ccccc2)c1OCc1ccccc1]

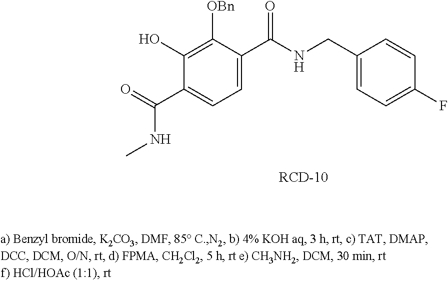 [SMILES: CNC(=O)c1ccc(C(=O)NCc2ccc(F)cc2)c(OCc2ccccc2)c1O]

### Dimethyl 2,3-bis(benzyloxy)terephthalate (13)

To a solution of 12 (1 g, 4.4 mmol) in 20 mL DMF was added K₂CO₃ (2.43 mg, 17.6 mmol) and benzyl bromide (120 μL, 10 mmol).

The mixture was refluxed for 10 h at 85° C., at which time the insoluble salts were filtered off. Approximately 10 mL of H₂O was added to the filtrate and the resulting off-white precipitate was collected. Yield=90%. ¹H NMR (400 MHz, CDCl₃, 25° C.): δ=4.49 (d, J=8.0 Hz, 4H), 6.86 (t, J=8.0 Hz, 4H; ArH), 7.16 (t, J=6.0 Hz, 4H; ArH), 7.99 (t, J=8.0 Hz, 1H; ArH), 8.32 (d, J=8.0 Hz, 2H; ArH), 8.37 (brt, J=8.0 Hz, 2H; NH). ESI-MS(+) m/z 381.99 [M+H]⁺.

### 2,3-Bis(benzyloxy)terephthalic acid (14)

To a solution of 13 (1.1 g, 2.7 mmol) in 60 mL THF was added 20 mL of 4% KOH/H₂O. The solution was stirred for 4 h at room temperature, after which 40 mL of water was added. The solution was then washed with EtOAc and acidified with 6M HCl until a precipitate formed. The product was isolated by filtration as a white solid. Yield=91%. ¹H NMR (400 MHz, DMSO-d₆, 25° C.): δ=5.02 (s, 4H), 7.33 (m, 6H; ArH), 7.39 (m, 4H; ArH), 7.48 (s, 2H; ArH). ESI-MS(−) m/z 376.83 [M−H]⁻.

### (2,3-Bis(benzyloxy)-1,4-phenylene)bis((2-thioxothiazolidin-3-yl)methanone) (15)

The synthesis of this compound was adapted from a literature procedure (Cohen, S. M.; Petoud, S.; et al. Inorg. Chem. 1999, 38, 4522) starting from 14 (800 mg, 2.11 mmol) and producing a yellow solid as the product. Yield=89%. ¹H NMR (400 MHz, CDCl₃, 25° C.): δ=2.95 (t, J=8.0 Hz, 4H), 4.31 (t, J=8.0 Hz, 4H), 5.07 (s, 4H), 7.20 (s, 2H; ArH), 7.35 (m, 10H; ArH). ESI-MS(+) m/z 580.74 [M+H]⁺.

### 2,3-Bis(benzyloxy)-N1-(4-fluorobenzyl)-N4-methylterephthalamide (16)

Compound 15 (1.1 g, 1.9 mmol) was combined with FPMA (80 μL, 0.7 mmol) in 120 mL of CH₂Cl₂. After 3 h, the reaction mixture was evaporated to dryness and partially purified by passage through a silica plug using 5% MeOH/CH₂Cl₂ as eluant. The semi-purified material was dissolved in 12 mL of CH₂Cl₂ to which 800 mL of CH₃NH₂ (40% aqueous solution) was added. After 30 min, the reaction mixture was evaporated to dryness and purified by silica column chromatography using 0-5% MeOH/CH₂Cl₂ as eluant. After removal of solvent the desired product was isolated as a white solid (343 mg, 0.69 mmol). Yield=98%. ¹H NMR (400 MHz, CDCl₃, 25° C.): δ=2.82 (d, J=4.0 Hz, 3H), 4.44 (d, J=8.0 Hz, 2H), 5.08 (d, J=4.0 Hz, 4H), 6.93 (t, J=8.0 Hz, 2H; ArH), 7.20 (m, 12H; ArH), 7.66 (brt, J=8.0 Hz, 1H; NH), 7.93 (q, J=8.0 Hz, 2H; ArH), 8.10 (brt, J=8.0 Hz, 1H; NH). ESI-MS(+) m/z 498.90 [M+H]⁺.

### N1-(4-Fluorobenzyl)-2,3-dihydroxy-N4-methylterephthalamide (RCD-10)

Compound 16 (340 mg, 0.68 mmol), was stirred in 18 mL of a 1:1 solution of HCl:HOAc at room temperature for 3 d to obtain a turbid mixture. Addition of water resulted in precipitation of a white solid that was isolated by filtration and washed with water (159 mg, 0.5 mmol). Yield=73%. ¹H NMR (400 MHz, DMSO-d₆, 25° C.): δ=2.80 (d, J=4.0 Hz, 3H), 4.47 (d, J=8.0 Hz, 2H), 7.15 (t, J=8.0 Hz, 2H; ArH), 7.33 (d, J=8.0 Hz, 2H; ArH), 7.36 (t, J=8.0 Hz, 2H; ArH), 8.87 (brt, J=4.0 Hz, 1H; NH), 9.36 (brt, J=4.0 Hz, 1H; NH). ESI-MS(+) m/z 318.96 [M+H]⁺. Anal. Calcd for C₁₆H₁₅FN₂O₄: C, 60.37; H, 4.75; N, 8.80. Found: C, 60.54; H, 4.79; N, 8.89.

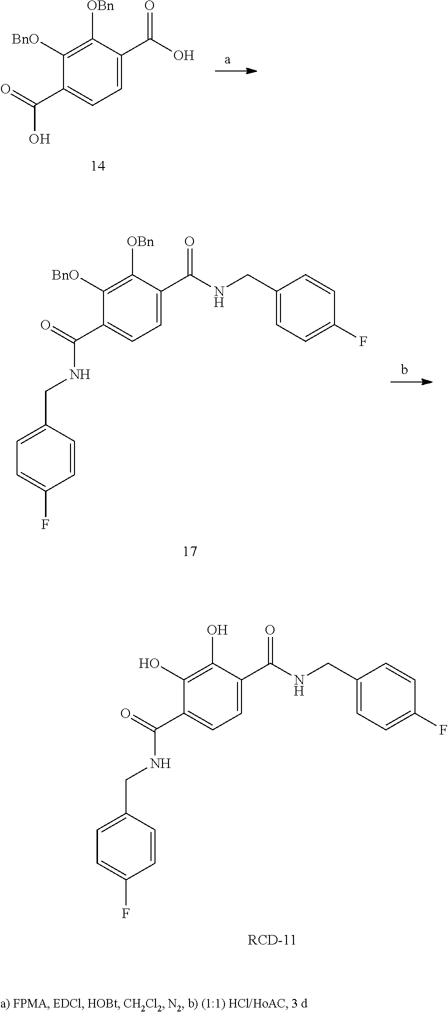 [SMILES: O=C(O)c1ccc(C(=O)O)c(OCc2ccccc2)c1OCc1ccccc1 ; O=C(NCc1ccc(F)cc1)c1ccc(C(=O)NCc2ccc(F)cc2)c(OCc2ccccc2)c1OCc1ccccc1 ; O=C(NCc1ccc(F)cc1)c1ccc(C(=O)NCc2ccc(F)cc2)c(O)c1O]

### 2,3-Bis(benzyloxy)-N1,N4-bis(4-fluorobenzyl)terephthalamide (17)

This compound was prepared from 14 (300 mg, 0.79 mmol) according to the procedure outlined for 32 (see below). The product was isolated as a white solid. Yield=54%. ¹H NMR (400 MHz, CDCl₃, 25° C.): δ=4.42 (d, J=10.0 Hz, 4H), 5.05 (s, 4H), 6.95 (t, J=8.0 Hz, 4H; ArH), 7.14 (m, 10H; ArH), 7.29 (t, J=8.0 Hz, 4H; ArH), 7.35 (s, 2H; ArH), 8.06 (brt, J=8.0 Hz, 2H; NH). ESI-MS(+) m/z 592.95 [M+H]⁺.

### N1,N4-Bis(4-fluorobenzyl)-2,3-dihydroxyterephthalamide (RCD-11)

Compound 17 (250 mg, 0.42 mmol), was stirred in 16 mL of a 1:1 solution of HCl:HOAc at room temperature for 3 d to obtain a turbid mixture. Addition of water resulted in precipitation of a white solid that was isolated by filtration and washed with water (143 mg, 0.35 mmol). Yield=83%. ¹H NMR (400 MHz, CDCl₃, 25° C.): δ=4.62 (d, J=4.0 Hz, 4H), 7.05 (t, J=8.0 Hz, 4H; ArH), 7.14 (s, 2H; ArH), 7.20 (brt, J=6.0 Hz, 2H; NH), 7.33 (t, J=6.0 Hz, 4H; ArH), 10.74 (brs, 2H, OH). ESI-MS(+) m/z 412.96 [M−H]⁺. Anal. Calcd for C₂₂H₁₈F₂N₂O₄: C, 64.07; H, 4.40; N, 6.79. Found: C, 63.87; H, 4.45; N, 6.89.

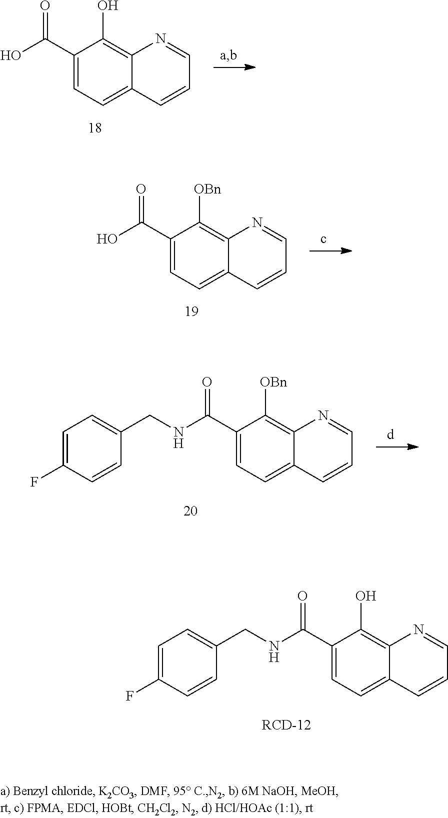 [SMILES: O=C(O)c1ccc2cccnc2c1O ; O=C(O)c1ccc2cccnc2c1OCc1ccccc1 ; O=C(NCc1ccc(F)cc1)c1ccc2cccnc2c1OCc1ccccc1 ; O=C(NCc1ccc(F)cc1)c1ccc2cccnc2c1O]

### 8-(Benzyloxy)quinoline-7-carboxylic acid (19)

To a solution of 8-hydroxyquinoline-7-carboxylic acid (18) (500 mg, 2.64 mmol) in 10 mL of DMF was added benzyl chloride (782 μL, 6.78 mmol) and K₂CO₃ (1.03 g, 7.47 mmol). The resulting mixture was heated to reflux at 120° C. under nitrogen and stirred overnight. The mixture was then vacuum filtered and the filtrate was concentrated under vacuum to a reddish-brown oil. The oil was purified via a silica plug using CH₂Cl₂ as eluant, after which removal of solvent gave an orange oil (585 mg, 1.58 mmol). Yield=60%. To a solution of the oil (585 mg, 1.58 mmol) in 5 mL of MeOH was added, 3 mL of 6M NaOH. The solution was stirred overnight at room temperature under nitrogen. The solution was then evaporated under vacuum and the residue was dissolved in CH₂Cl₂ and washed with 6M HCl. The organic phase was collected, dried over anhydrous MgSO₄, and concentrated under vacuum to give a yellow solid (444 mg, 1.58 mmol). Yield=99%. ¹H NMR (400 MHz, DMSO-d₆, 25° C.): δ=5.43 (s, 2H; CH₂), 7.33 (d, J=7.2 Hz, 2H; ArH), 7.37 (t, J=7.2 Hz, 2H; ArH), 7.58 (d, J=6.8 Hz, 2H; ArH), 7.63 (dd, J=8.4, 4.4 Hz, 1H; ArH), 7.77 (d, J=2.4 Hz, 1H; ArH), 8.42 (dd, J=8.4, 1.4 Hz, 1H; ArH), 9.01 (dd, J=4.4, 2.0 Hz, 1H; ArH). ESI-MS(−) m/z 278.32 [M−H]⁻.

### 8-(Benzyloxy)-N-(4-fluorobenzyl)quinoline-7-carboxamide (20)

To a solution of 19 (400 mg, 1.43 mmol) in 15 mL of dry CH₂Cl₂ was added EDCI (329 mg, 1.72 mmol), HOBt (232 mg, 1.72 mmol), and FPMA (197 μL, 1.72 mmol). The mixture was stirred overnight at room temperature under nitrogen, after which the solution was extracted with CH₂Cl₂ and washed with 1M HCl. The organic phase was collected, dried over anhydrous MgSO₄, and concentrated under vacuum to give a yellow oil. The oil was purified via silica column chromatography using 0-2% MeOH/CH₂Cl₂ as eluant. After removal of solvent the product was obtained as a yellow solid (171 mg, 0.44 mmol). Yield=31%. ¹H NMR (400 MHz, CDCl₃, 25° C.): δ=4.43 (d, J=5.6 Hz, 2H; NHCH₂), 5.51 (s, 2H; CH₂), 6.92 (t, J=8.8 Hz, 2H; ArH), 7.13 (dd, J=6.4, 3.0 Hz, 2H; ArH), 7.32 (d, J=5.2 Hz, 5H; ArH), 7.48 (dd, J=8.4, 4.0 Hz, 1H; ArH), 7.64 (d, J=8.4 Hz, 1H; ArH), 8.18 (dd, J=8.4, 2.0, Hz, 1H; ArH), 8.28 (d, J=8.8 Hz, 1H; ArH), 8.60 (brt, 1H; CONHCH₂), 8.99 (dd, J=4.0, 1.6 Hz, 1H; ArH). ESI-MS(+) m/z 387.11 [M+H]⁺.

### N-(4-Fluorobenzyl)-2,3-dihydroxybenzamide (RCD-12)

Compound 20 (154 mg, 0.40 mmol) was stirred in 10 mL of a 1:1 was stirred in 25 mL of a 1:1 solution of HCl:HOAc at room temperature for 5 d to obtain a turbid mixture. The solution was evaporated to dryness and the resulting residue was co-evaporated with 3×5 mL of MeOH and the resulting solid was dried overnight in a vacuum oven to yield the product as a yellow solid (101 mg, 0.34 mmol). Yield=85%. ¹H NMR (500 MHz, DMSO-d₆, 25° C.): δ=4.54 (d, J=4.6 Hz, 2H; NHCH₂), 7.13 (t, J=8.6 Hz, 2H; ArH), 7.38 (t, J=6.0 Hz, 2H; ArH), 7.57 (d, J=9.1, Hz, 1H; ArH), 7.85 (brt, 1H; ArH), 8.17 (d, J=8.6 Hz, 1H; ArH), 8.67 (d, J=8.0 Hz, 1H; ArH), 9.00 (brs, 1H; ArH), 9.75 (brt, 1H; CONHCH₂. ¹³C NMR (125 MHz, DMSO-d₆, 25° C.): 42.4 (CH₂), 113.6 (ArC), 115.5 (ArC), 115.7 (ArC), 117.7 (ArC), 124.4 (ArC), 126.1 (ArC), 130.0 (ArC), 131.4 (ArC), 135.4 (ArC), 148.2 (ArC), 155.9 (ArC), 160.7 (ArC), 162.7 (ArC), 168.8 (C═O). ESI-MS(+) m/z 297.12 [M+H]⁺. Anal. Calcd for C₁₇H₁₃FN₂O₂.2.25H₂O: C, 60.62; H, 5.24; N, 8.32. Found: C, 60.53; H, 4.83; N, 8.33.

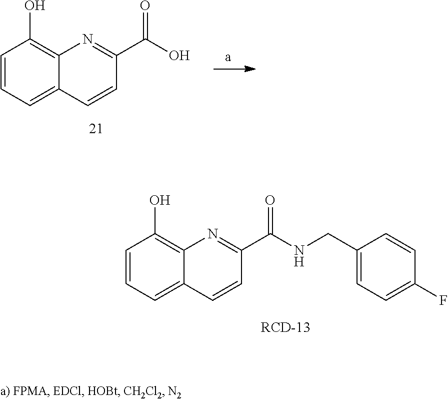 [SMILES: O=C(O)c1ccc2cccc(O)c2n1 ; O=C(NCc1ccc(F)cc1)c1ccc2cccc(O)c2n1]

### N-(4-Fluorobenzyl)-8-hydroxyquinoline-2-carboxamide (RCD-13)

To a solution of 8-hydroxyquinoline-2-carboxylic acid, (21, 400 mg, 2.1 mmol) in 20 mL of CH₂Cl₂ was added EDCI (487 mg, 2.5 mmol), HOBt (343 mg, 2.5 mmol), and FPMA (290 μL, 2.5 mmol). The resulting mixture was stirred at room temperature for 16 h under nitrogen. The mixture was washed with 1M HCl and brine. The organic phase was collected and dried over anhydrous MgSO₄. The crude product was evaporated under vacuum and purified via flash silica column chromatography using 0-5% MeOH/CH₂Cl₂ as eluant to give the product as a pale yellow solid (383 mg, 1.3 mmol). Yield=61%. ¹H NMR (400 MHz, DMSO-d₆, 25° C.): δ=4.59 (d, J=8.0 Hz, 2H), 7.17 (t, J=8.0 Hz, 2H; ArH), 7.19 (d, J=8.0 Hz, 1H; ArH), 7.40 (t, J=4.0 Hz, 2H; ArH), 7.46 (d, J=8.0 Hz, 1H; ArH), 7.55 (t, J=8.0 Hz, 1H; ArH), 8.15 (d, J=8.0 Hz, 1H; ArH), 8.49 (d, J=8.0 Hz, 1H; ArH), 10.14 (brt, J=8.0 Hz, 1H; NH). ESI-MS(+) m/z 297.09 [M+H]⁺. Anal. Calcd for C₁₇H₁₃FN₂O₂: C, 68.91; H, 4.42; N, 9.45. Found: C, 68.99; H, 4.81; N, 9.56.

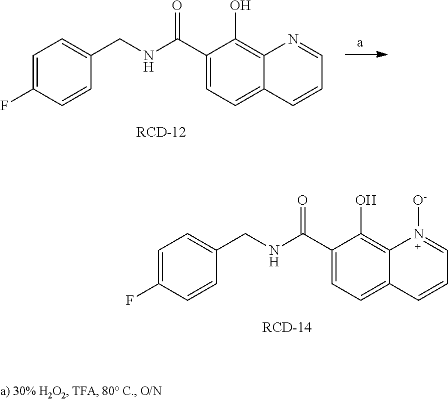

### 7-((4-Fluorobenzyl)carbamoyl)-8-hydroxyquinoline 1-oxide (RCD-14)

This compound was prepared from RCD-12 as adapted from a literature procedure (Agrawal, A. et al. J. Med. Chem. 2009, 52, 1063); a detailed procedure is provided for RCD-16 (see below). RCD-12 (183 mg, 0.5 mmol) was combined with TFA and H₂O₂ to produce a dark brown solid. Yield=35%. ¹H NMR (400 MHz, CDCl₃, 25° C.): δ=4.72 (d, J=8.0 Hz, 2H), 7.05 (t, J=8.0 Hz, 2H; ArH), 7.39 (m, 3H; ArH), 7.61 (dd, J=8.0 Hz, J=4.0 Hz, 1H; ArH), 8.17 (d, J=8.0 Hz, 1H; ArH), 8.24 (d, J=8.0 Hz, 1H), 8.27 (br, 1H; NH), 8.89 (d, J=4.0 Hz, 1H). ESI-MS(+) m/z 296.97 [M-O-]⁺. Anal. Calcd for C₁₇H₁₃FN₂O₃: C, 63.19; H, 4.43; N, 8.67. Found: C, 63.42; H, 4.85; N, 8.17.

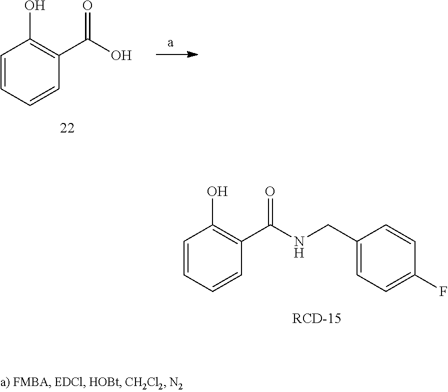 [SMILES: O=C(O)c1ccccc1O ; O=C(NCc1ccc(F)cc1)c1ccccc1O]

### N-(4-Fluorobenzyl)-2-hydroxybenzamide (RCD-15)

To a solution of 2-hydroxybenzoic acid (22, 500 mg, 3.6 mmol) in 20 mL of CH₂Cl₂ was added EDCI (833 mg, 4.3 mmol), HOBt (585 mg, 4.3 mmol), and FPMA (495 μL, 4.3 mmol). The mixture was stirred at room temperature for 16 h under nitrogen. The reaction was then rinsed with 1M HCl and brine. The organic phase was collected and dried over anhydrous MgSO₄. The crude product was evaporated under vacuum and purified via flash silica column chromatography using CH₂Cl₂ as eluant, which after removal of solvent gave the product as a white solid (302 mg, 1.2 mmol). Yield=34%. ¹H NMR (400 MHz, DMSO-d₆, 25° C.): δ=4.48 (d, J=4.0 Hz, 2H), 6.88 (t, J=8.0 Hz, 2H; ArH), 7.13 (t, J=8.0 Hz, 2H; ArH), 7.38 (m, 3H; ArH), 7.86 (d, J=8.0 Hz, 1H; ArH), 9.34 (brt, J=8.0 Hz, 1H; NH). ESI-MS(+) m/z 245.99 [M+H]⁺. Anal. Calcd for C₁₄H₁₂FNO₂: C, 68.56; H, 4.93; N, 5.71. Found: C, 68.18; H, 5.35; N, 5.87.

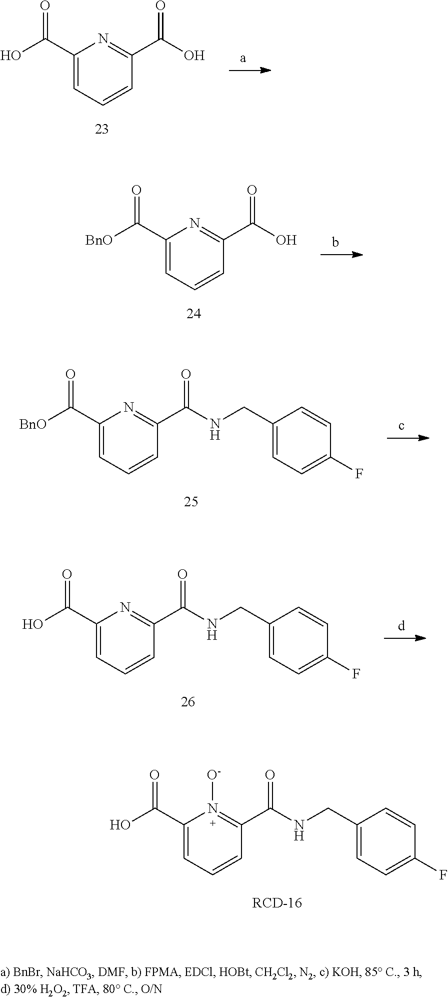

### 6-((Benzyloxy)carbonyl)picolinic acid (24)

The synthesis of this compound was adapted from a literature procedure (Gardiner, J. et al. Chem. Biodiversity, 2006, 3, 1181). To pyridine-2,6-dicarboxylic acid (23, 2 g, 12 mmol) in 40 mL DMF was added NaHCO₃ (1.18 g, 14.4 mmol) and benzyl bromide (1.7 mL, 14.4 mmol). The reaction mixture was heated to 60° C. for 16 h, after which the solution was cooled to room temperature. To the reaction, 40 mL of H₂O was added and the aqueous layer was rinsed with EtOAc before being acidified to pH 3 with 1M HCl. The solution was extracted with EtOAc, the organic phase was collected, dried over anhydrous MgSO₄, and the crude mixture was evaporated under vacuum to give a white solid. Yield=16%. ¹H NMR (400 MHz, DMSO-d₆, 25° C.): δ=5.41 (s, 2H), 7.45 (m, 5H; ArH), 8.22 (m, 3H; ArH). ESI-MS(−) m/z 255.92 [M−H]⁻.

### N-(4-Fluorobenzyl)-6-(2-phenylacetyl)picolinamide (25)

This compound was prepared according to the coupling procedure outlined for compound 8. Compound 24 (400 mg, 1.56 mmol) was combined with 1.2 eq of FPMA to give the desired product (58 mg, 0.52 mmol). Yield=10%. ¹H NMR (400 MHz, CDCl₃, 25° C.): δ=4.65 (d, J=8.0 Hz, 2H), 5.43 (s, 2H), 7.02 (t, J=8.0 Hz, 2H; ArH), 7.36 (m, 7H; ArH), 8.01 (t, J=8.0 Hz, 1H; ArH), 8.22 (d, J=8.0 Hz, 1H; ArH), 8.40 (d, J=8.0 Hz, 1H; ArH), 8.54 (brt, J=8.0 Hz, 1H; NH). ESI-MS(+) m/z 364.90 [M+H]⁺.

### 6-((4-fluorobenzyl)carbamoyl)picolinic acid (26)

To a solution of 25 (300 mg, 0.82 mmol) in 20 mL MeOH was added KOH (157 mg, 2.8 mmol). The reaction mixture was heated to 85° C. for 4 h, then neutralized with HCl. The solvent was removed under vacuum and the resulting solid was dissolved in 5% MeOH/CH₂Cl₂. Insoluble particles were hot filtered and the solution was dried under vacuum to produce a white solid. Yield=90%. ¹H NMR (400 MHz, CDCl₃, 25° C.): δ=4.32 (d, J=4.0 Hz, 2H), 6.72 (t, J=8.0 Hz, 2H; ArH), 7.01 (t, J=6.0 Hz, 2H; ArH), 7.54 (brt, J=6.0 Hz, 1H; NH), 7.87 (d, J=8.0 Hz, 1H; ArH), 7.95 (d, J=4.0 Hz, 1H), 8.82 (brt, J=6.0 Hz, 1H; NH). ESI-MS(−) m/z 272.90 [M−H]⁻.

### 2-Carboxy-6-((4-fluorobenzyl)carbamoyl)pyridine 1-oxide (RCD-16)

The synthesis of this compound was adapted from a literature procedure (Agrawal, A. et al. J. Med. Chem. 2009, 52, 1063). A mixture of 1.5 mL TFA and 220 μL of 30% H₂O₂ was added to 26 (100 mg, 0.55 mmol). The solution was refluxed at 80° C. for 16 h, and then cooled to room temperature. Approximately 7 mL of water was added and the brown precipitate that formed was filtered off and collected. Yield=19%. ¹H NMR (400 MHz, CDCl₃, 25° C.): δ=4.67 (d, J=4.0 Hz, 2H), 7.06 (t, J=8.0 Hz, 2H; ArH), 7.35 (t, J=6.0 Hz, 2H; ArH), 7.82 (t, J=8.0 Hz, 1H; ArH), 8.60 (dd, J=8.0 Hz, J=4.0 Hz, 1H; ArH), 8.74 (dd, J=8.0 Hz, J=4.0 Hz, 1H; ArH), 10.29 (brt, J=8.0 Hz, 1H; NH). ESI-MS(−) m/z 288.65 [M−H]⁻. Anal. Calcd for C₁₄H₁₁FN₂O₄: C, 57.93; H, 3.82; N, 9.65. Found: C, 58.19; H, 4.10; N, 9.37.

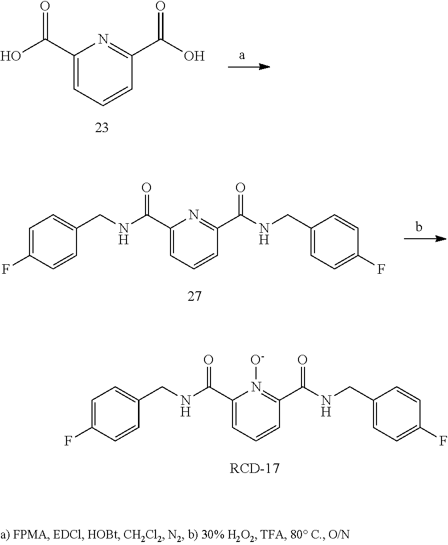

### N2,N6-Bis(4-fluorobenzyl)pyridine-2,6-dicarboxamide (27)

To a solution 23 (400 mg, 2.4 mmol) in 15 mL of CH₂Cl₂ was added EDCI (1 g, 5.3 mmol), HOBt (712 mg, 5.3 mmol), and FPMA (620 μL, 5.3 mmol). The mixture was stirred at room temperature for 16 h under nitrogen. The reaction was then washed with 1M HCl and brine. The organic phase was collected and dried over anhydrous MgSO₄. The crude product was evaporated under vacuum and purified via flash silica column chromatography using 0-5% MeOH/CH₂Cl₂ as eluant. Yield=65%. ¹H NMR (400 MHz, CDCl₃, 25° C.): δ=4.49 (d, J=8.0 Hz, 4H), 6.86 (t, J=8.0 Hz, 4H; ArH), 7.16 (t, J=6.0 Hz, 4H; ArH), 7.99 (t, J=8.0 Hz, 1H; ArH), 8.32 (d, J=8.0 Hz, 2H; ArH), 8.37 (brt, J=8.0 Hz, 2H; NH). ESI-MS(+) m/z 381.99 [M+H]⁺.

### 2,6-Bis((4-fluorobenzyl)carbamoyl)pyridine 1-oxide (RCD-17)

RCD-17 was prepared according to the procedure outlined for RCD-16 using 27 (580 mg, 1.5 mmol) as the starting material. The desired compound was purified via flash silica column chromatography using 0-5% MeOH/CH₂Cl₂ as the eluant. Yield=10%. ¹H NMR (300 MHz, CDCl₃, 25° C.): δ=4.63 (d, J=6.0 Hz, 4H), 7.03 (t, J=9.0 Hz, 4H; ArH), 7.33 (t, J=7.5 Hz, 4H; ArH), 7.62 (t, J=6.0 Hz, 1H; ArH), 8.60 (d, J=6.0 Hz, 2H; ArH), 10.93 (br, 2H; NH). ESI-MS(+) m/z 397.98 [M+H]⁺. Anal. Calcd for C₂₁H₁₇F₂N₃O₃: C, 63.47; H, 4.31; N, 10.57. Found: C, 63.10; H, 4.40; N, 10.72.

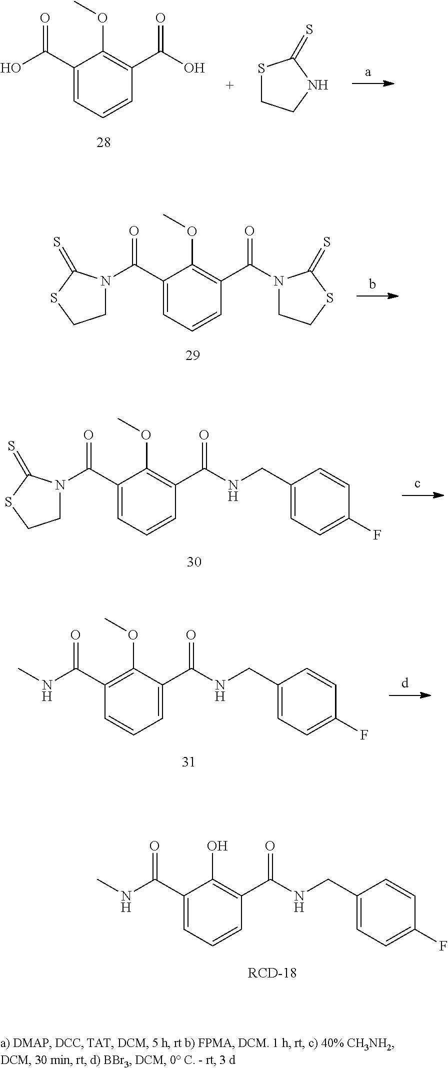 [SMILES: COc1c(C(=O)O)cccc1C(=O)O ; S=C1NCCS1 ; COc1c(C(=O)N2CCSC2=S)cccc1C(=O)N1CCSC1=S ; COc1c(C(=O)NCc2ccc(F)cc2)cccc1C(=O)N1CCSC1=S ; CNC(=O)c1cccc(C(=O)NCc2ccc(F)cc2)c1OC ; CNC(=O)c1cccc(C(=O)NCc2ccc(F)cc2)c1O]

### (2-Methoxy-1,3-phenylene)bis((2-thioxothiazolidin-3-yl)methanone) (29)

The synthesis of this compound was adapted from a literature procedure (Cohen, S. M. et al. Inorg. Chem. 1999, 38, 4522). To a solution of 2-methoxyisophthalic acid (2.7 g, 13.8 mmol) (28) in 120 mL of CH₂Cl₂ was added thiazolidine-2-thione (3.3 g, 28 mmol), a catalytic amount of N,N-dimethylaminopyridine (DMAP), and N,N′-dicyclohexylcarbodiimide (DCC, 5.7 g, 28 mmol) at room temperature. The mixture was stirred for 5 h under nitrogen. The solution was then filtered and the solvent was removed from the filtrate under vacuum. The compound was purified via flash silica column chromatography using CH₂Cl₂ as eluant to give the product as a bright yellow solid (1.6 g, 3.9 mmol). Yield=29%. ¹H NMR (400 MHz, CDCl₃, 25° C.): δ=3.42 (t, J=8.0 Hz, 4H), 3.90 (s, 3H), 4.60 (t, J=8.0 Hz, 4H), 7.14 (t, J=8.0 Hz, 1H; ArH), 7.43 (d, J=4.0 Hz, 2H; ArH). ESI-MS(+) m/z 398.67 [M+H]⁺.

### N-(4-Fluorobenzyl)-2-methoxy-3-(2-thioxothiazolidine-3-carbonyl)benzamide (30)

The synthesis of this compound was adapted from literature procedure (Cohen, S. M. et al. Inorg. Chem. 1999, 38, 4522). To a solution of 29 (300 mg, 0.73 mmol) in 100 mL of CH₂Cl₂ was added FPMA (29 μL, 0.24 mmol). The reaction mixture was stirred overnight at room temperature under nitrogen. The solvent was then removed under vacuum and the resulting mixture was purified via flash silica column chromatography using CH₂Cl₂ as eluant to give the product as a bright yellow solid. Yield=88%. ¹H NMR (400 MHz, CDCl₃, 25° C.): δ=3.43 (t, J=6.0 Hz, 2H), 3.75 (s, 3H), 4.60 (d, J=4.0 Hz, 2H), 4.65 (t, J=8.0 Hz, 2H), 7.02 (t, J=8.0 Hz, 2H; ArH), 7.21 (t, J=8.0 Hz, 1H; ArH), 7.25 (t, J=4.0 Hz, 2H; ArH), 7.33 (d, J=8.0 Hz, 1H; ArH), 7.80 (brt, J=8.0 Hz, 1H; NH), 8.15 (d, J=8.0 Hz, 1H; ArH). ESI-MS(+) m/z 404.81 [M+H]⁺.

### N1-(4-Fluorobenzyl)-2-methoxy-N3-methylisophthalamide (31)

To a solution of 30 (150 mg, 0.37 mmol) in 4 mL CH₂Cl₂ was added 230 μL, of CH₃NH₂ (40% aqueous solution) at room temperature. The mixture was stirred vigorously for 30 min under nitrogen. The solution was washed with water and the crude material was purified via flash silica column chromatography using 0-10% MeOH/CH₂Cl₂ as eluant. Yield=76%. ¹H NMR (400 MHz, CDCl₃, 25° C.): δ=2.93 (d, J=4.0 Hz, 3H), 3.69 (s, 3H), 4.55 (d, J=4.0 Hz, 2H), 6.99 (t, J=8.0 Hz, 2H; ArH), 7.22 (t, J=8.0 Hz, 1H; ArH), 7.29 (t, J=8.0 Hz, 2H; ArH), 7.75 (brt, J=8.0 Hz, 1H; NH), 7.94 (d, J=8.0 Hz, 1H; ArH), 7.98 (d, J=9.0 Hz, 1H; ArH). ESI-MS(+) m/z 317.0 [M+H]⁺.

### N1-(4-Fluorobenzyl)-2-hydroxy-N3-methylisophthalamide (RCD-18)

RCD-18 was prepared according to the detailed procedure outlined for RCD-19 (see below) and was isolated as a white solid (30.6 mg, 0.10 mmol). Yield=36%. ¹H NMR (400 MHz, CDCl₃, 25° C.): δ=3.01 (d, J=4.0 Hz, 3H), 4.61 (d, J=4.0 Hz, 2H), 6.87 (t, J=8.0 Hz, 1H; ArH), 7.01 (t, J=8.0 Hz, 2H; ArH), 7.30 (t, J=6.0 Hz, 2H; ArH), 7.61 (br, 1H; NH), 7.90 (d, j=8.0 Hz, 1H), 8.05 (d, J=8.0 Hz, 1H; ArH), 8.29 (br, 1H; NH). ESI-MS(+) m/z 302.95 [M+H]⁺. Anal. Calcd for C₁₆H₁₄FNO₄: C, 63.57; H, 5.00; N, 9.27. Found: C, 63.32; H, 5.10; N, 9.28.

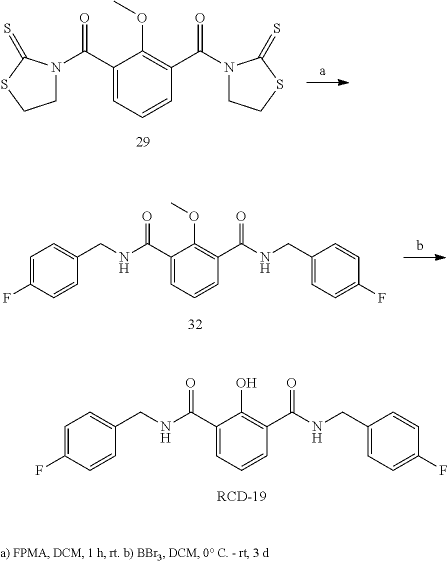 [SMILES: COc1c(C(=O)N2CCSC2=S)cccc1C(=O)N1CCSC1=S ; COc1c(C(=O)NCc2ccc(F)cc2)cccc1C(=O)NCc1ccc(F)cc1 ; O=C(NCc1ccc(F)cc1)c1cccc(C(=O)NCc2ccc(F)cc2)c1O]

### N1,N3-Bis(4-fluorobenzyl)-2-methoxyisophthalamide (32)

To a solution of 29 (300 mg, 0.75 mmol) in 100 mL CH₂Cl₂ was added FPMA (215 μL, 1.87 mmol). The reaction was stirred at room temperature overnight. The solvent was then removed under vacuum and the crude product was purified via flash silica column chromatography with CH₂Cl₂ as eluant. Yield=23%. ¹H NMR (400 MHz, CDCl₃, 25° C.): δ=3.43 (t, J=6.0 Hz, 2H), 3.75 (s, 3H), 4.60 (d, J=8.0 Hz, 2H), 4.64 (t, J=6.0 Hz, 2H), 7.03 (t, J=8.0 Hz, 2H; ArH), 7.24 (t, J=8.0 Hz, 1H; ArH), 7.31 (t, J=8.0 Hz, 2H; ArH), 7.41 (d, J=8.0 Hz, 1H; ArH), 7.76 (brt, J=8.0 Hz, 1H; NH), 8.16 (d, J=8.0 Hz, 1H; ArH). ESI-MS(+) m/z 404.79 [M+H]⁺.

### N1,N3-Bis(4-fluorobenzyl)-2-hydroxyisophthalamide (RCD-19)

To a solution of 32 (70 mg, 0.17 mmol) in 15 mL CH₂Cl₂ was added BBr₃ (58 mg, 0.23 mmol) under nitrogen at 0° C. The mixture was stirred for 3 d, the reaction was then quenched with MeOH, and the mixture was diluted with water. The solution was boiled until the yellow color dissipated and the volume of the solution was reduced by half. MeOH was added to induce precipitation and the resulting white solid was isolated by filtration. Yield=21%. ¹H NMR (400 MHz, CDCl₃, 25° C.): δ=4.64 (d, J=4.0 Hz, 4H), 6.97 (t, J=8.0 Hz, 1H; ArH), 7.04 (t, J=8.0 Hz, 4H; ArH), 7.33 (t, J=6.0 Hz, 4H; ArH), 7.70 (br, 2H; NH), 7.97 (d, J=8.0 Hz, 2H). ESI-MS(+) m/z 396.93 [M+H]⁺. Anal. Calcd for C₂₂H₁₈F₂N₂O₃: C, 66.66; H, 4.58; N, 7.07. Found: C, 66.54; H, 4.98; N, 6.86.

In Vitro Integrase Catalytic Assays

Recombinant HIV-1 IN and oligonucleotide substrates were obtained as previously reported (Marinello et al. Biochemistry 2008, 47, 9345-9354; Metifiot et al. Antimicrob. Agents Chemother. 2011, 55, 5127-5133; Hare et al. Mol. Pharmacol. 2011, 80, 565-572). Integrase reactions were performed in 10 μL total volume including 400 nM HIV-1 IN, 20 nM 5′-end [³²P]-labeled oligonucleotide substrate, and 1 μL inhibitor solution in 50 mM MOPS, pH 7.2, 7.5 mM MgCl₂, and 14.3 mM 2-mercaptoethanol Inhibitor dilutions were in DMSO, and DMSO without drug was used as a control. Reactions were incubated at 37° C. for 60 min, terminated by adding 10 μL loading dye (10 mM EDTA, 98% deionized formamide, 0.025% xylene cyanol, and 0.025% bromophenol blue), and were subjected to electrophoresis in 20% polyacrylamide-7 M urea gels. Gels were dried and reaction products were visualized and quantified with a Typhoon 8600 (GE Healthcare, Little Chalfont, Buckinghamshire, UK). Densitometric analyses were performed using ImageQuant from Molecular Dynamics Inc. The concentrations at which enzyme activity was reduced by 50% (IC₅₀) were determined using “Prism” software (GraphPad Software, San Diego, Calif.) for nonlinear regression to fit dose-response data to logistic curve models.

Computational Docking Studies

The coordinates for the X-ray crystal structure of PFV-IN were taken from the RCSB Protein Data Bank (entry: 3OYA) and prepared using the Protein Preparation Wizard, which is a part of the Maestro software package (Maestro v9.1; Schrodinger, Inc.). The Protein Preparation Wizard was used to add bond order assignments and formal charges for heterogroups (amino acid residues, metal-ligand bonds) and hydrogen atoms to the system. To optimize the hydrogen bonding network histidine tautomers and ionization states were predicted, and manual corrections were made when necessary to ensure correct coordination with the two Mg (II) ions. Proper assignment of Asn and Gln sidechains was assessed by rotating 180° around the terminal χ angle of these residues while adding hydrogen atoms to sample the hydrogen-bonding network around the residues to determine if the oxygen and nitrogen atoms were properly assigned. All water molecules in the structure were removed.

Three-dimensional structures of the RCD fragments and Raltegravir were prepared using LigPrep (LigPrep v2.4 Schrodinger, Inc.) with Epik (Epik v2.1 Schrodinger, Inc.) to generate multiple protonation and tautomeric states for the ligands at pH values of 7.0±2.0.

The metal binding state (i.e. deprotonated hydroxyl groups) of the RCD compounds were docked flexibly into the active site of the prepared PFV-IN structure. Docking was preformed with Glide 5.5 (Glide v5.5; Schrodinger, Inc.) with the standard precision scoring function to estimate protein-ligand binding affinities. A maximum of ten scoring poses were saved for each fragment. The top scoring poses for each fragment were found to possess the expected binding modes with reasonable metal-ligand bond distances based on the 3OYA crystal complex.

To calculate the RMSD of the various compounds, the superposition tool within Maestro was used. The two compounds of interest were selected and the atoms to be compared were manually selected to generate the RMSD value. The calculations were conducted using the ‘in place’ option, which omits a post-docking minimization of the compounds that is designed to move the structures in order get the lowest possible RMS difference between the two superimposed fragments.

### V. Embodiments

### Embodiment 1

A compound having the formula:

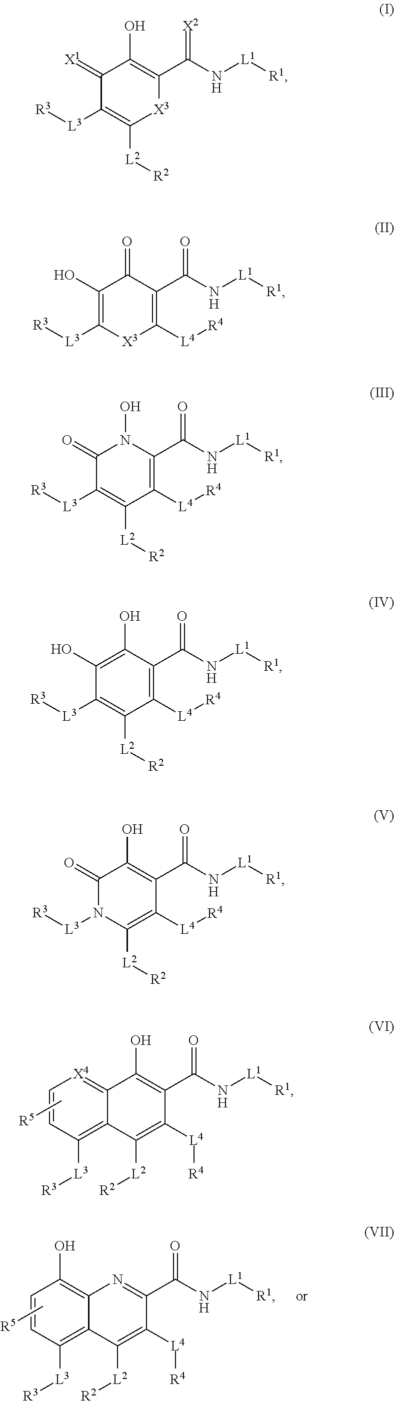

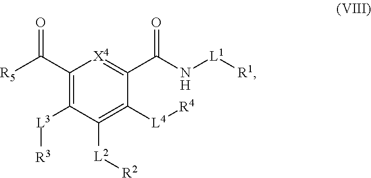

wherein, X¹ and X² are, independently ═O or ═S; X³ is —O—, or —N(-L⁴-R⁴)—; X^{3′} is —O—, or —N(-L²-R²)—; X⁴ is —C(OH)═, —N═, or —N⁺(O)═; R¹, R², R³, and R⁴ are, independently, hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁵ is hydrogen, —OR⁶, —NHR⁷, —SO₂NR⁸, —C(O)NR⁹, —C(O)—OR¹⁰, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R⁶, R⁷, R⁸, R⁹, and R¹⁰ are independently hydrogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and L¹, L², L³ and L⁴ are independently a bond, —S(O)—, —S(O)₂NH—, —NHS(O)₂—, —C(O)O—, —OC(O)—, —C(O)—, —C(O)NH—, —NH—, —NHC(O)—, —O—, —S—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

### Embodiment 2

The compound of embodiment 1, wherein the compound has the structure of Formula (I).

### Embodiment 3

The compound of embodiment 1, wherein the compound has the structure of Formula (II).

### Embodiment 4

The compound of embodiment 1, wherein the compound has the structure of Formula (III).

### Embodiment 5

The compound of embodiment 1, wherein the compound has the structure of Formula (IV).

### Embodiment 6

The compound of embodiment 1, wherein the compound has the structure of Formula (V).

### Embodiment 7

The compound of embodiment 1, wherein the compound has the structure of Formula (VI).

### Embodiment 8

The compound of embodiment 1, wherein the compound has the structure of Formula (VII).

### Embodiment 9

The compound of embodiment 1, wherein the compound has the structure of Formula (VIII).

### Embodiment 10

The compound as in any one of embodiments 1-9, wherein R¹, R², R³, and R⁴ are, independently, hydrogen, substituted or unsubstituted C₁-C₂₀ alkyl, substituted or unsubstituted 2 to 20 membered heteroalkyl, C₃-C₈ cykloalkyl, substituted or unsubstituted 3 to 8 membered heterocycloalkyl, substituted or unsubstituted C₅-C₁₀ aryl, or substituted or unsubstituted 5 to 10 membered heteroaryl.

### Embodiment 11

The compound of embodiment 10, wherein R¹ is substituted or unsubstituted C₅-C₁₀ aryl.

### Embodiment 12

The compound of embodiment 11, wherein R¹ is substituted or unsubstituted phenyl.

### Embodiment 13

The compound of embodiment 12, wherein R¹ is halophenyl.

### Embodiment 14

The compound of embodiment 10, wherein R² is substituted or unsubstituted 5 to 10 membered heteroaryl.

### Embodiment 15

The compound of embodiment 14, wherein R² is substituted 5 to 10 membered heteroaryl.

### Embodiment 16

The compound of embodiment 14, wherein R² is substituted oxadiazolyl.

### Embodiment 17

The compound of embodiment 10, wherein R², R³, and R⁴ are, independently substituted or unsubstituted C₁-C₁₀ alkyl.

### Embodiment 18

The compound of embodiment 17, wherein R², R³, and R⁴ are, independently unsubstituted C₁-C₄ alkyl.

### Embodiment 19

The compound of embodiment 18, wherein R², R³, and R⁴ are, independently methyl or ethyl.

### Embodiment 20

The compound of embodiment 10, wherein R², R³, and R⁴ are, independently hydrogen.

### Embodiment 21

The compound as in any one of embodiments 1-9, wherein R⁵ is —OR⁶ or —NHR⁷.

### Embodiment 22

The compound of embodiment 21, wherein R⁶ is hydrogen.

### Embodiment 23

The compound of embodiment 21, wherein R⁵ is —NHR⁷.

### Embodiment 24

The compound of embodiment 23, wherein R⁷ is hydrogen, substituted or unsubstituted C₁-C₂₀ alkyl, substituted or unsubstituted 2 to 20 membered heteroalkyl, C₃-C₈ cykloalkyl, substituted or unsubstituted 3 to 8 membered heterocycloalkyl, substituted or unsubstituted C₅-C₁₀ aryl, or substituted or unsubstituted 5 to 10 membered heteroaryl.

### Embodiment 25

The compound of embodiment 24, wherein R⁷ is substituted or unsubstituted C₁-C₁₀ alkyl.

### Embodiment 26

The compound of embodiment 25, wherein R⁷ is unsubstituted C₁-C₄ alkyl.

### Embodiment 27

The compound of embodiment 26, wherein R⁷ is methyl or ethyl.

### Embodiment 28

The compound as in any one of embodiments 1-9, wherein L¹, L², L³ and L⁴ are, independently a bond, —C(O)NH—, substituted or unsubstituted C₁-C₁₀ alkylene, or substituted or unsubstituted 2 to 10 membered heteroalkylene.

### Embodiment 29

The compound of embodiment 28, wherein L¹, L³ and L⁴ are a bond.

### Embodiment 30

The compound of embodiment 28, wherein L¹, L³ and L⁴ are independently unsubstituted C₁-C₁₀ alkylene.

### Embodiment 31

The compound of embodiment 30, wherein L¹, L³ and L⁴ are methylene.

### Embodiment 32

The compound of embodiment 28, wherein L³ is —C(O)NH—.

### Embodiment 33

The compound of embodiment 28, wherein L² is substituted or unsubstituted 2 to 6 membered heteroalkylene.

### Embodiment 34

The compound as in any one of embodiments 1-9, wherein L²-R² is having the formula:

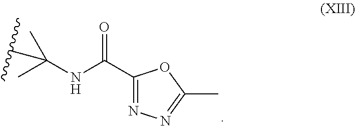 [SMILES: *C(C)(C)NC(=O)c1nnc(C)o1]

### Embodiment 35

The compound as in any one of embodiments 1-9, wherein R³ is hydrogen and L³ is a bond.

### Embodiment 36

The compound as in any one of embodiments 1-9, wherein R⁴ is hydrogen and L⁴ is a bond.

### Embodiment 37

The compound of embodiment 1 having the structure of Formula (II), wherein L¹ is a bond; R¹ is halophenyl; X^{3′} is —N(-L²-R²); L²-R² is

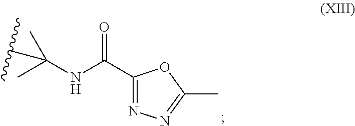 [SMILES: *C(C)(C)NC(=O)c1nnc(C)o1]

L³ is a bond; R³ is hydrogen; L⁴ is a bond; and R⁴ is methyl.

### Embodiment 38

The compound of embodiment 1 having the structure of Formula (IV), wherein L¹ is a bond; R¹ is halophenyl; L²-R² is

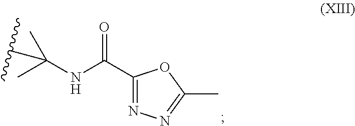 [SMILES: *C(C)(C)NC(=O)c1nnc(C)o1]

L³ is —C(O)NH—; R³ is methyl; L⁴ is a bond; and R⁴ is hydrogen.

### Embodiment 39

A pharmaceutical composition comprising a pharmaceutically acceptable excipient and a compound of any one of embodiments 1-38.

### Embodiment 40

A method of treating an infectious disease in a subject in need thereof, said method comprising administering to said subject a therapeutically effective amount of a compound of any one of embodiments 1-38.

### Embodiment 41

The method of embodiment 40, wherein said infectious disease is caused by a virus.

### Embodiment 42

The method of embodiment 41, wherein said virus is HIV

### Embodiment 43

The method of embodiment 40, wherein said subject suffers from AIDS.

### Embodiment 44

A method of inhibiting HIV integrase in a patient, said method comprising administering to said patient a therapeutically effective amount of a compound of any one of embodiments 1-38 thereby inhibiting HIV integrase in said patient.

### Embodiment 45

A method of inhibiting HIV integrase, said method comprising contacting HIV integrase with an effective amount of a compound of any one of embodiments 1-38 thereby inhibiting said HIV integrase.

### VI. Tables

TABLE 1
Assay results for RCD compounds against the 3′-processing (3P) and strand
transfer (ST) reactions of HIV-1 IN, as well as inhibition of viral replication. The chelate ring
sizes formed upon binding the active site metal ions is also indicated.

| | Structure (MBG) | | | | |
|---|---|---|---|---|---|
| Compound | 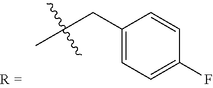 [SMILES: *Cc1ccc(F)cc1] | Chelate Ring Size (Mg_{A}, Mg_{B}) | 3'-Processing IC₅₀ (μM) | Strand Transfer IC₅₀ (μM) | Antiviral Activity IC₅₀ (μM) |
| RCD-1 | 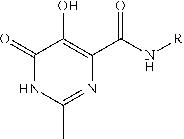 | 5-, 6- | >100 | 1.0 ± 0.3 | 1.5 |
| RCD-2 | 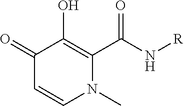 | 5-, 6- | >100 | >100 | n.d. |
| RCD-3 | 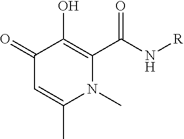 | 5-, 6- | >100 | >100 | n.d. |
| RCD-4 | 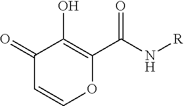 | 5-, 6- | >100 | 0.96 ± 0.3 | n.d. |
| RCD-4S | 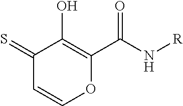 | 5-, 6- | >100 | 11.5 ± 0.9 | n.d. |
| RCD-4S² | 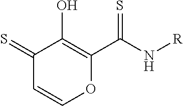 | 5-, 6- | 64 ± 6 | 7.3 ± 0.6 | n.d. |
| RCD-5 | 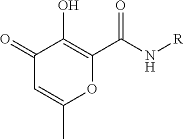 | 5-, 6- | 59.5 ± 1.4 | 0.55 ± 0.1 | 1.0 |
| RCD-6 | 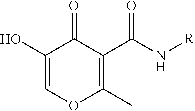 | 5-, 6- | >100 | 56.0 ± 7.0 | n.d. |
| RCD-7 | 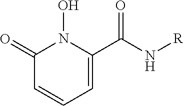 | 5-, 6- | >100 | 19.7 ± 1.6 | n.d. |
| RCD-8 | 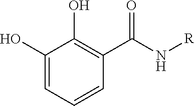 | 5-, 6- | >100 | 39.4 ± 4.0 | n.d. |
| RCD-9 | 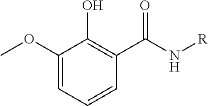 | 5-, 6- | >100 | >100 | n.d. |
| RCD-10 | 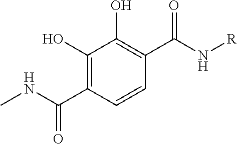 | 5-, 6- | >200 | 1.5 ± 0.2 | 4.0 |
| RCD-11 | 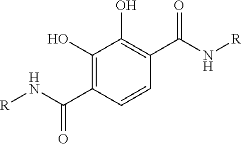 | 5-, 6- | >300 | 1.7 ± 0.2 | n.d. |
| RCD-12 | 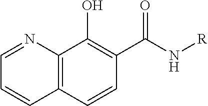 | 5-, 6- | >100 | 14.5 ± 2.2 | 2.3* |
| RCD-13 | 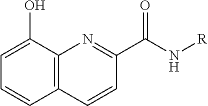 | 5-, 5- | >100 | >100 | >100 |
| RCD-14 | 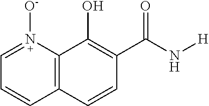 | 6-, 6- | 40.5 ± 2.0 | 3.8 ± 0.3 | 0.5* |
| RCD-15 | 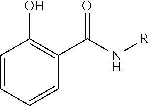 | NA, 6- | >100 | >100 | n.d. |
| RCD-16 | 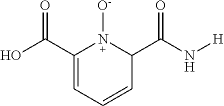 [SMILES: NC(=O)C1C=CC=C(C(=O)O)N1[O-]] | 6-, 6- | 21.4 ± 3.0 | 9.2 ± 1.3 | n.d. |
| RCD-17 | 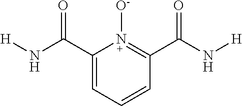 | 6-, 6- | >300 | >100 | >100 |
| RCD-18 | 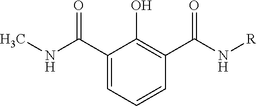 | 6-, 6- | >300 | >300 | >100 |
| RCD-19 | 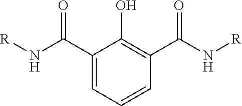 | 6-, 6- | >300 | >300 | n.d. |

*Compound showed some cellular toxicity at 10 μM.

TABLE 2
RCD compounds according to the embodiments provided herein
and having the potential ability to inhibit the 3′-processing
(3P) and strand transfer (ST) reactions of HIV-1
IN, as well as inhibition of viral replication.

| | |
|---|---|
| 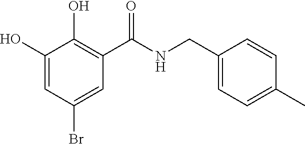 [SMILES: Cc1ccc(CNC(=O)c2cc(Br)cc(O)c2O)cc1] | RCD-20 |
| 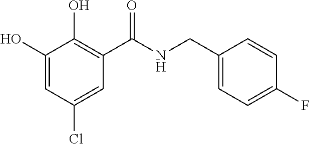 [SMILES: O=C(NCc1ccc(F)cc1)c1cc(Cl)cc(O)c1O] | RCD-21 |
| 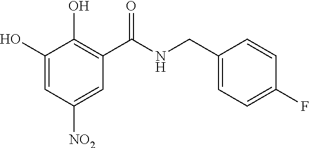 [SMILES: O=C(NCc1ccc(F)cc1)c1cc([N+](=O)[O-])cc(O)c1O] | RCD-22 |
| 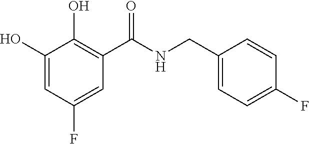 [SMILES: O=C(NCc1ccc(F)cc1)c1cc(F)cc(O)c1O] | RCD-23 |
| 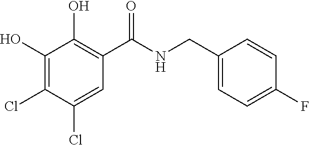 [SMILES: O=C(NCc1ccc(F)cc1)c1cc(Cl)c(Cl)c(O)c1O] | RCD-24 |
| 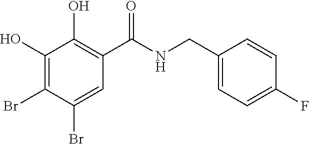 [SMILES: O=C(NCc1ccc(F)cc1)c1cc(Br)c(Br)c(O)c1O] | RCD-25 |
| 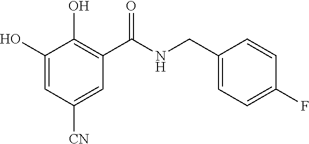 [SMILES: N#Cc1cc(O)c(O)c(C(=O)NCc2ccc(F)cc2)c1] | RCD-26 |
| 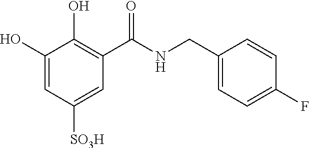 [SMILES: O=C(NCc1ccc(F)cc1)c1cc(S(=O)(=O)O)cc(O)c1O] | RCD-27 |
| 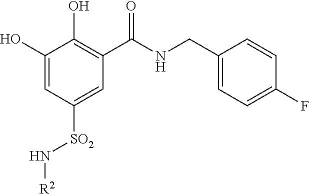 | RCD-28 |
| 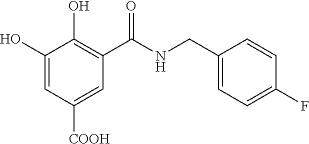 [SMILES: O=C(O)c1cc(O)c(O)c(C(=O)NCc2ccc(F)cc2)c1] | RCD-29 |
| 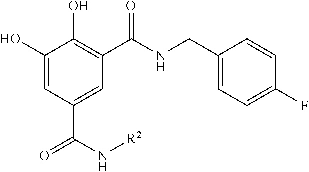 | RCD-30 |
| 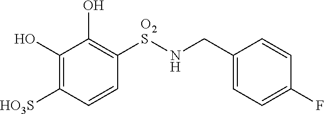 [SMILES: O=S(=O)(O)c1ccc(S(=O)(=O)NCc2ccc(F)cc2)c(O)c1O] | RCD-31 |
| 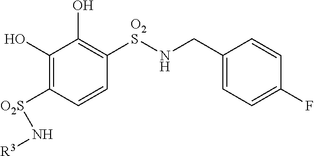 | RCD-32 |
| 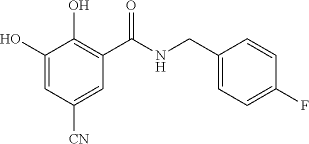 [SMILES: N#Cc1cc(O)c(O)c(C(=O)NCc2ccc(F)cc2)c1] | RCD-33 |
| 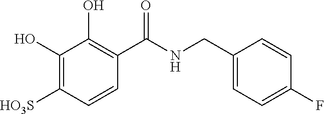 [SMILES: O=C(NCc1ccc(F)cc1)c1ccc(S(=O)(=O)O)c(O)c1O] | RCD-34 |
| 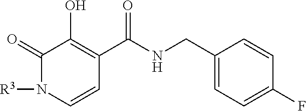 | RCD-35 |
| 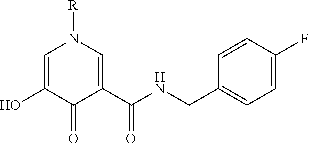 | RCD-36 |

1. A compound having the formula: 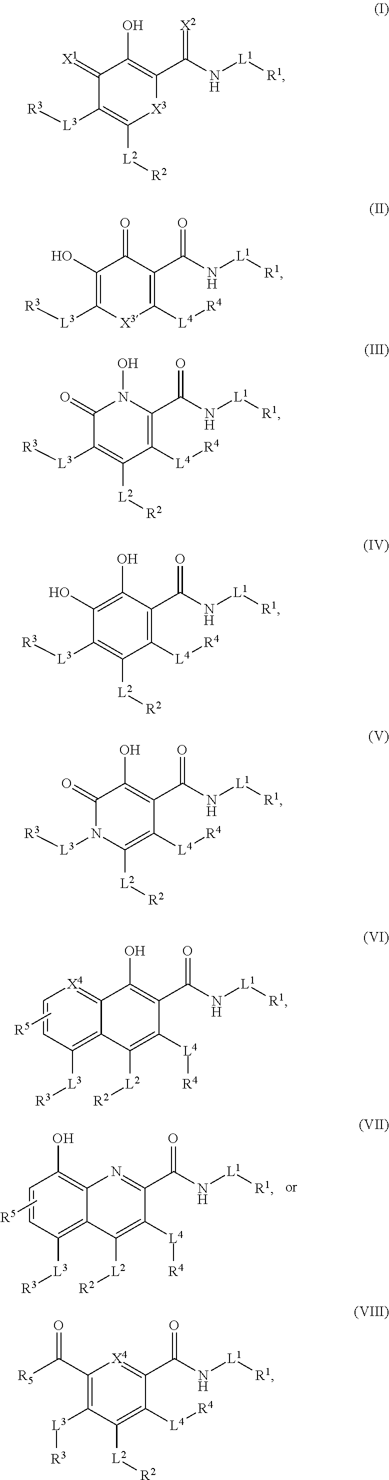
wherein,
X¹ and X² are, independently ═O or ═S;
X³ is —O—, or —N(-L⁴-R⁴)—;
X^{3′} is —O—, or —N(-L²-R²)—;
X⁴ is —C(OH)—, —N═, or —N⁺(O)═;
R¹, R², R³, and R⁴ are independently, hydrogen, halogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁵ is hydrogen, —OR⁶, —NHR⁷, —SO₂NR⁸, —C(O)NR⁹, —C(O)—OR¹⁰, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
R⁶, R⁷, R⁸, R⁹, and R¹⁰ are independently hydrogen, —CF₃, —CN, —CCl₃, —COOH, —CH₂COOH, —CONH₂, —OH, —SH, —SO₂Cl, —SO₃H, —SO₄H, —SO₂NH₂, —NO₂, —NH₂, —NHNH₂, —ONH₂, —NHC═(O)NHNH₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
L¹, L², L³ and L⁴ are independently a bond, —S(O)—, —S(O)₂NH—, —NHS(O)₂—, —C(O)O—, —OC(O)—C(O)—, —C(O)NH—, —NH—, —NHC(O)—, —O—, —S —, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

2. (canceled)

3. (canceled)

4. (canceled)

5. (canceled)

6. (canceled)

7. (canceled)

8. (canceled)

9. (canceled)

10. The compound of claim 1, wherein R¹, R², R³, and R⁴ are, independently, hydrogen, substituted or unsubstituted C₁-C₂₀ alkyl, substituted or unsubstituted 2 to 20 membered heteroalkyl, C₃-C₈ cykloalkyl, substituted or unsubstituted 3 to 8 membered heterocycloalkyl, substituted or unsubstituted aryl, or substituted or unsubstituted 5 to 10 membered heteroaryl.

11. The compound of claim 10, wherein R¹ is substituted or unsubstituted C₅-C₁₀ aryl.

12. (canceled)

13. The compound of claim 11, wherein R¹ is halophenyl.

14. The compound of claim 10, wherein R² is substituted or unsubstituted 5 to 10 membered heteroaryl.

15. (canceled)

16. (canceled)

17. The compound of claim 10, wherein R², R³, and R⁴ are,
independently substituted or unsubstituted C₁-C₁₀ alkyl.

18. (canceled)

19. (canceled)

20. The compound of claim 10, wherein R², R³, and R⁴ are,
independently hydrogen.

21. The compound of claim 1, wherein R⁵ is —OR⁶ or —NHR⁷.

22. (canceled)

23. The compound of claim 21, wherein R⁵ is —NHR⁷.

24. The compound of claim 23, wherein R⁷ is hydrogen, substituted or unsubstituted C₁-C₂₀ alkyl, substituted or unsubstituted 2 to 20 membered heteroalkyl, C₃-C₈ cykloalkyl, substituted or unsubstituted 3 to 8 membered heterocycloalkyl, substituted or unsubstituted C₅-C₁₀ aryl, or substituted or unsubstituted 5 to 10 membered heteroaryl.

25. (canceled)

26. (canceled)

27. (canceled)

28. The compound of claim 1, wherein L¹, L², L³ and L⁴ are, independently a bond, —C(O)NH—, substituted or unsubstituted C₁-C₁₀ alkylene, or substituted or unsubstituted 2 to 10 membered heteroalkylene.

29. (canceled)

30. (canceled)

31. The compound of claim 28, wherein L¹, L³ and L⁴ are methylene.

32. The compound of claim 28, wherein L³ is —C(O)NH—.

33. The compound of claim 28, wherein L² is substituted or unsubstituted 2 to 6 membered heteroalkylene.

34. The compound of claim 1, wherein L²-R² has the formula:

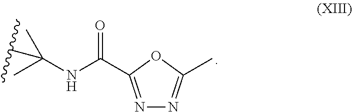 [SMILES: *C(C)(C)NC(=O)c1nnc(C)o1]

35. The compound of claim 1, wherein R³ is hydrogen and L³ is a bond.

36. The compound of claim 1, wherein R⁴ is hydrogen and L⁴ is a bond.

37. (canceled)

38. (canceled)

39. A pharmaceutical composition comprising a pharmaceutically acceptable excipient and a compound of claim 1.

40. A method of treating an infectious disease in a subject in need thereof, said method comprising administering to said subject a therapeutically effective amount of a compound of claim 1.

41. (canceled)

42. (canceled)

43. (canceled)

44. A method of inhibiting HIV integrase in a patient,
said method comprising administering to said patient a therapeutically effective amount of a compound of claim 1 thereby inhibiting HIV integrase in said patient.

45. (canceled)
